(19) United States
(12) Patent Application Publication
(10) Pub. No.: US 2026/0166176 A1
(43) Pub. Date: Jun. 18, 2026

(54) POLYMER-NUCLEIC ACID NANOPARTICLES FOR GENE EDITING

(71) Applicant: The Johns Hopkins University, Baltimore, MD (US)

(72) Inventors: Jordan J. GREEN, Woodstock, MD (US); Stephany Yi TZENG, Baltimore, MD (US); Garry R. CUTTING, Towson, MD (US); Erin KAVANAGH, Baltimore, MD (US)

(21) Appl. No.: 19/122,923

(22) PCT Filed: Nov. 6, 2023
(86) PCT No.: PCT/US2023/078797

§ 371 Date: Apr. 21, 2025

Related U.S. Application Data
(60) Provisional application No. 63/382,410, filed on Nov. 4, 2022.

(51) Int. Cl. A61K 48/00; C12N 9/22; C12N 15/11

(52) U.S. Cl. CPC A61K 48/0041; A61K 48/0058; C12N 9/226; C12N 15/11; C12N 2310/20

(57) ABSTRACT

Alterations of the 5′GT splice motif or 3′AG splice motif result in severe missplicing and are prevalent in many genetic diseases, including cystic fibrosis (CF). While protein-targeted modulator therapies are currently available for treatment of CF, individuals with these canonical splice site variants (CSSVs) are among the ˜10% who remain untreated. The most common CF-causing variant in individuals of African descent is a CSSV, c.2988+1G>A. While >75% of individuals with a CSSV have a modulator eligible in trans allele, only 50% of the 450 individuals bearing c.2988+1G>A are eligible. Thus, there is a particular unmet need for a treatment for these individuals. CRISPR/Cas9-mediated adenine base editing (ABE) is an efficient and targeted genome editing method correct G>A variants. We electroporated NRCH-ABE8e mRNA and a previously optimized sgRNA to non-differentiated human primary nasal (HNE) or bronchial (HBE) epithelial cells from individuals with CF compound heterozygous for c.2988+1G>A. After differentiation of edited cells genomic editing and recovery of CFTR channel function were assessed. In primary HBEs and primary HNEs, we observed an allelic conversion to WT of 74.7% and 81.3%, respectively, at the +1 site. Interestingly, we also observed high levels of editing at adjacent adenines (+3, +7), which would have a modest effect on mRNA splicing (˜20% reduction). However, this did not preclude recovery of CFTR channel function. Compared to WT/WT HBEs and HNEs, unedited cells showed ˜5% function, while edited cells achieved >50% function. Since electroporation is not a translationally viable delivery approach, we investigated polymeric nanoparticle mediated delivery to both primary HBE and HNE cells by flow cytometry. GFP mRNA evaluated transfection efficiency and cell viability across three dosages (150, 75, 32.5 ng), four polymer-to-mRNA weight-to-weight ratios (60, 40, 30, 20) and three polymers (R, X, Y). HBEs transfected with polymers R & Y showed ˜57% transfection at 75 and 32.5 ng, with polymer X averaging ˜25% across the same dosages. Polymer Y showed slightly higher viability of HBEs vs. polymer R. HNEs in comparison achieved a maximum of ˜37% GFP transfection with polymers R & Y at 32.5 ng compared to a maximum of ˜22% with polymer X at the same dose, with 75-95% viability across all polymers. The ABE design reported here corrects c.2988+1G>A in airway epithelia with high efficiency when robust delivery is achieved. Given that nanoparticle optimization allowed successful delivery to >50% of cells, we anticipate clinically significant recovery of function in vivo by combining this ABE design with an optimized polymeric nanoparticle.

### STATEMENT OF GOVERNMENTAL INTEREST

This invention was made with government support under grants AI160738 awarded by the National Institutes of Health. The government has certain rights in the invention.

### BACKGROUND

Current treatments for cystic fibrosis (CF) primarily focus on modulator drug therapies designed to correct malfunctioning cystic fibrosis transmembrane conductance regulator (CFTR) protein. Cutting, 2015. These modulators, however, are ineffective for the 10% of CF patients with variants that introduce a premature stop codon (PTC). Such variants include 3120+1 G>A, which introduces a PTC and results in loss of protein. Gene editing could allow production of CFTR protein and enhancement of function using available CFTR modulators. Sharma, 2018. The development of a safe and efficient non-viral gene delivery system for gene editing could dramatically advance the field and ultimately benefit CF patients.

### SUMMARY

In some aspects, the presently disclosed subject matter provides a nanoparticle comprising at least one DNA or mRNA molecule comprising a nucleic acid sequence encoding a gene-editing protein and a polymer of formula (I):

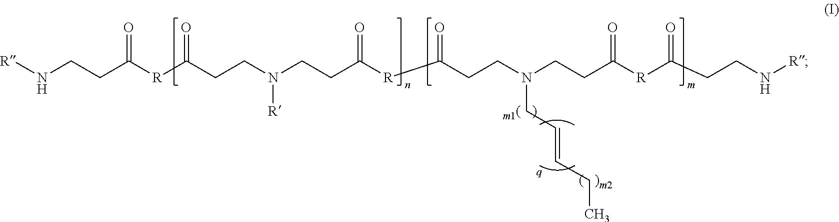

wherein:
m and n are each independently an integer from 1 to 10,000;
m1 is an integer selected from 0, 1, 2, 3, 4, 5, 6, 7, 8, 9, and 10;
m2 is an integer selected from the group consisting of 1, 2, 3, 4, 5, 6, 7, 8, 9, 10, 11, 12, 13, 14, 15, 16, 17, 18, 19, and 20;
q is an integer selected from 0 or 1;
   wherein:
—(CH₂)ₘ₁—(C═C)_{q}—(CH₂)ₘ₂—CH₃ comprises a hydrophobic sidechain;
R comprises a divalent radical comprising a biodegradable ester linkage and/or a bioreducible disulfide linkage;
R′ is hydrophilic sidechain comprising a monovalent radical derived from a hydrophilic amine monomer;
R″ is monovalent radical derived from an amine-containing end capping group; and
pharmaceutically acceptable salts thereof.

In certain aspects, R is selected from:

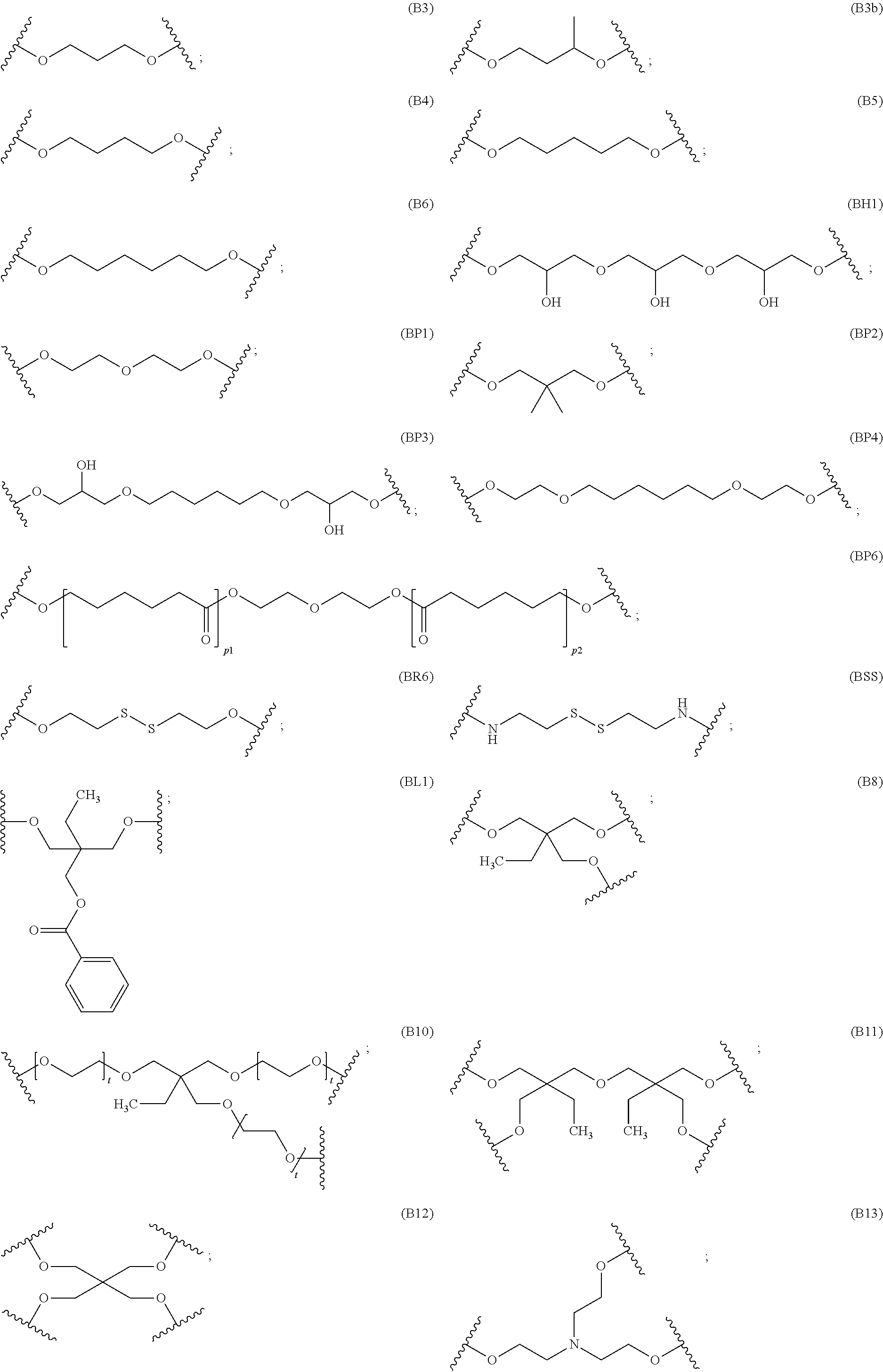

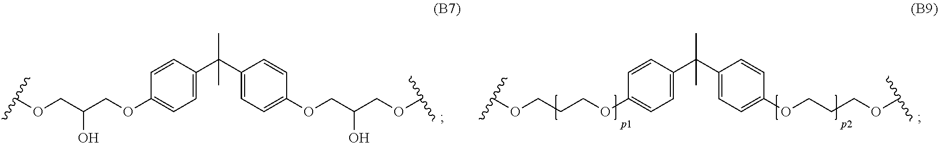

wherein each p1, p2, and t is independently an integer selected from the group consisting of 1, 2, 3, 4, 5, 6, 7, 8, 9, and 10.

In certain aspects, R′ is selected from:

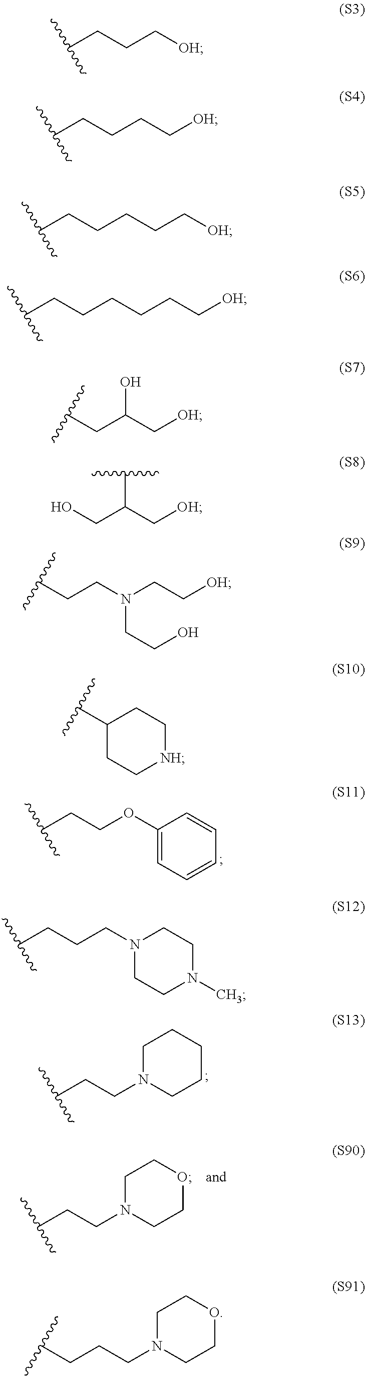

In certain aspects, R″ is selected from:

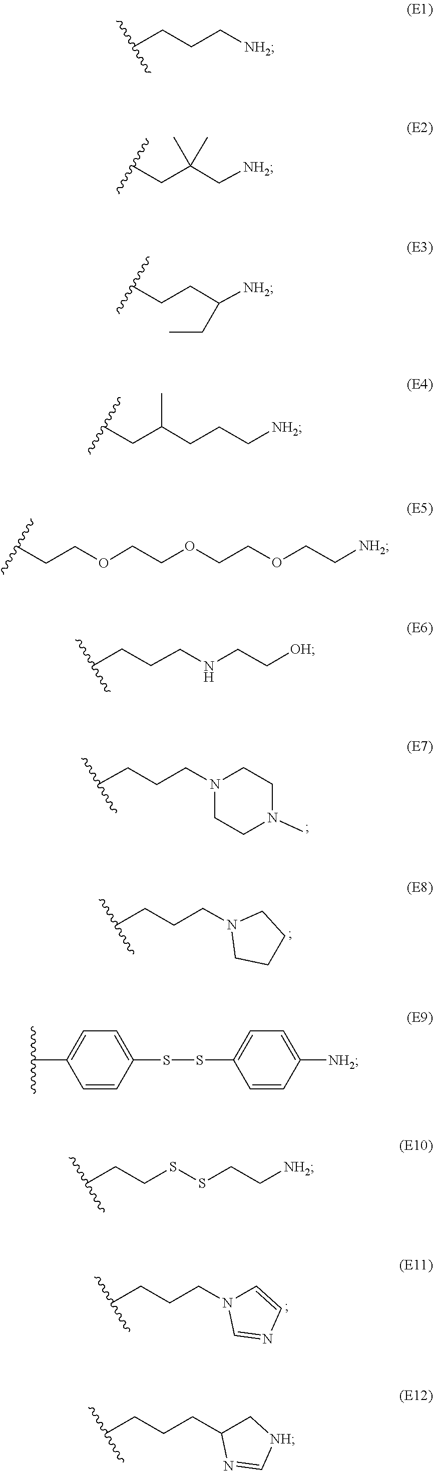

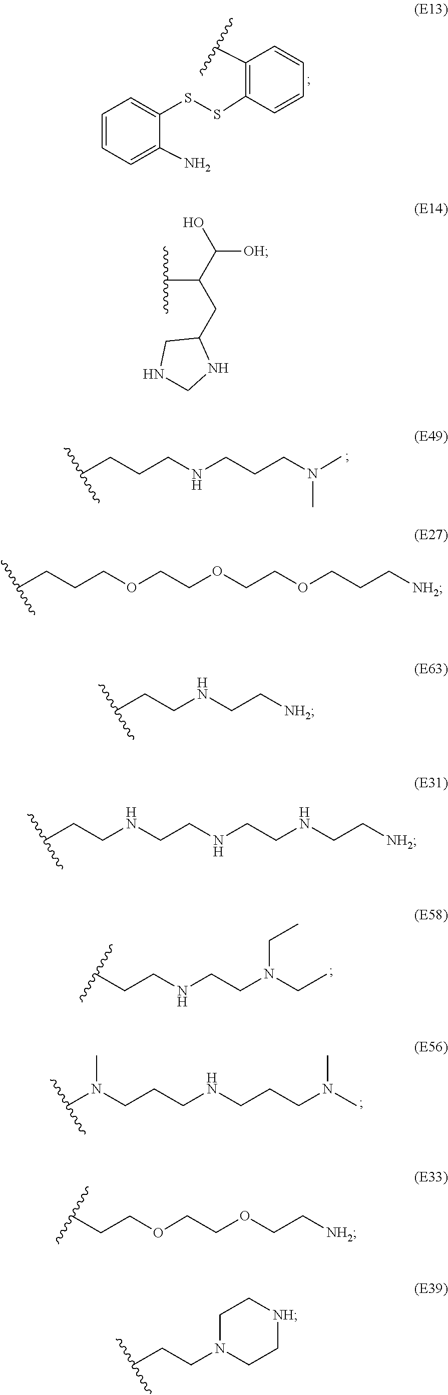

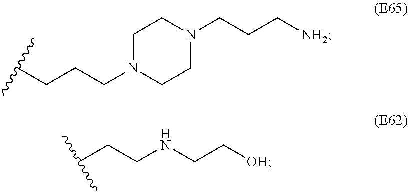 [SMILES: *CCCN1CCN(CCCN)CC1 ; *CCNCCO]

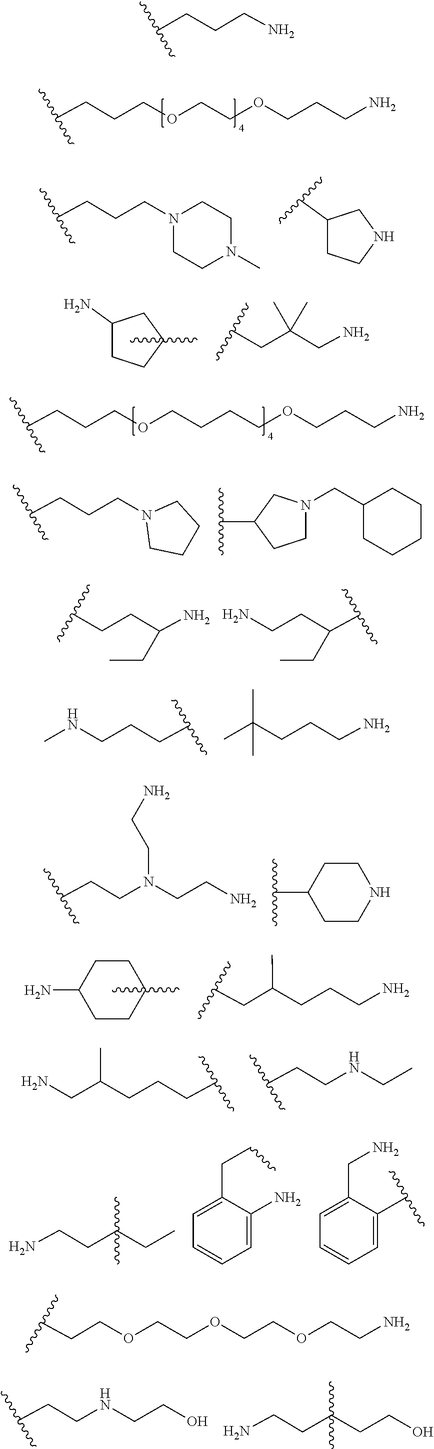

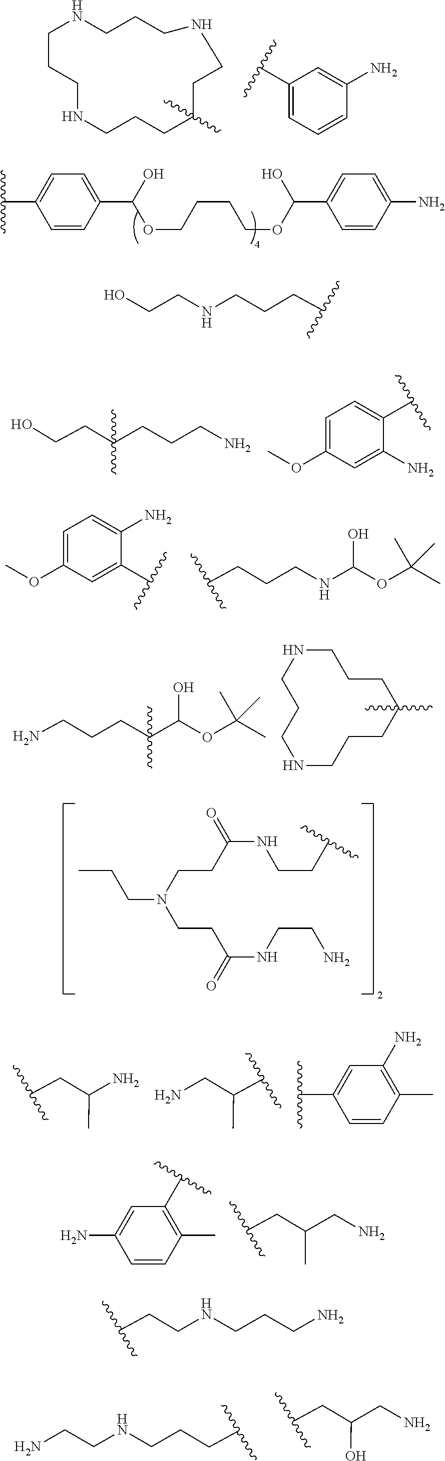

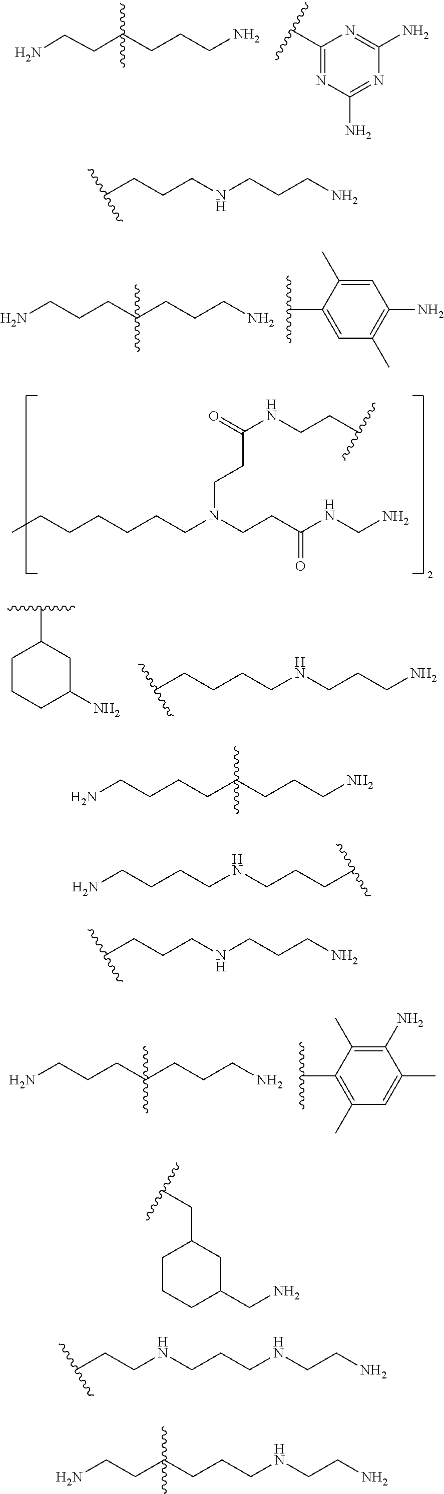

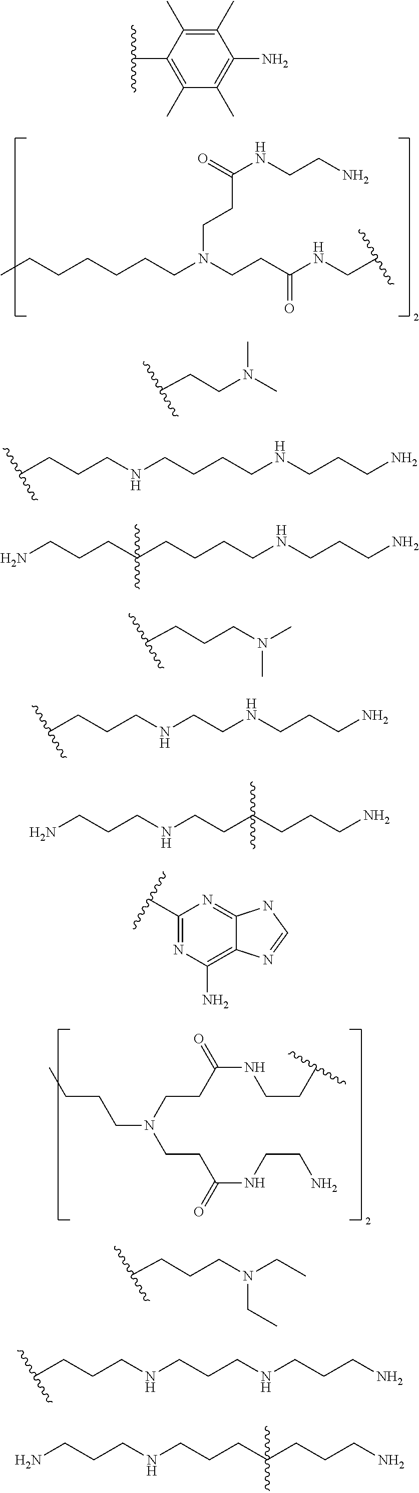

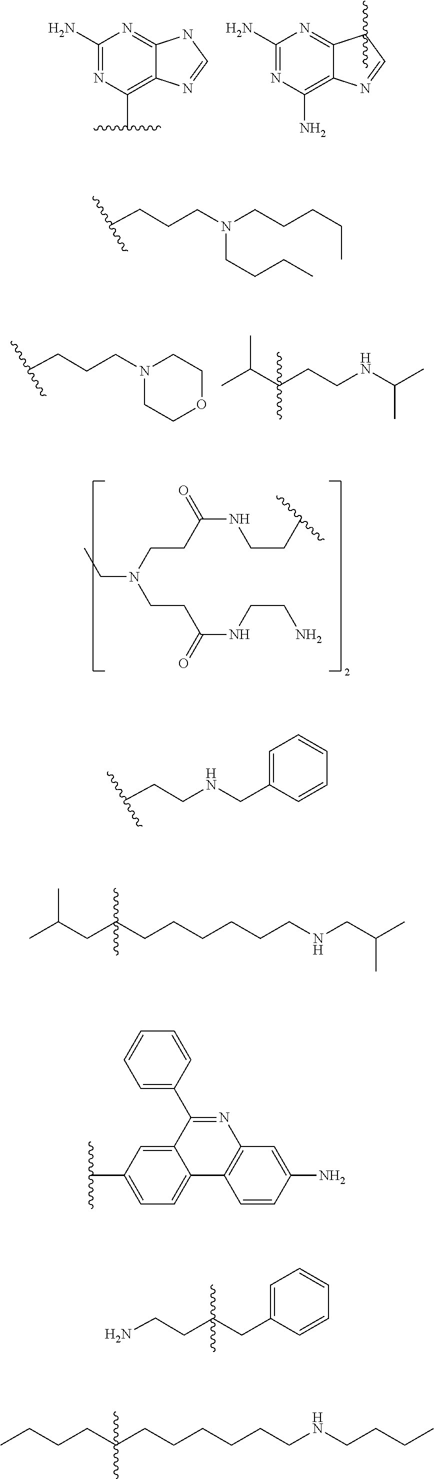

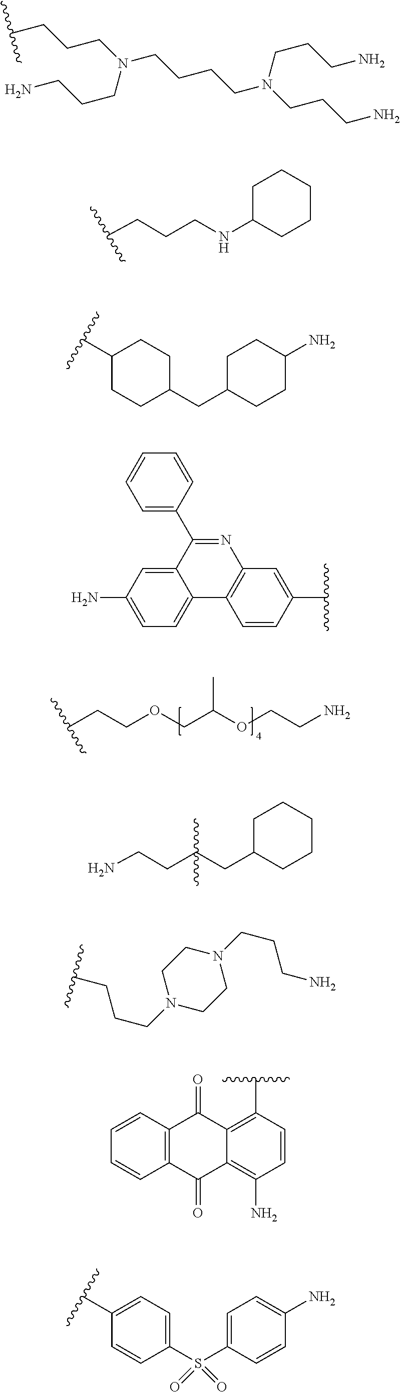

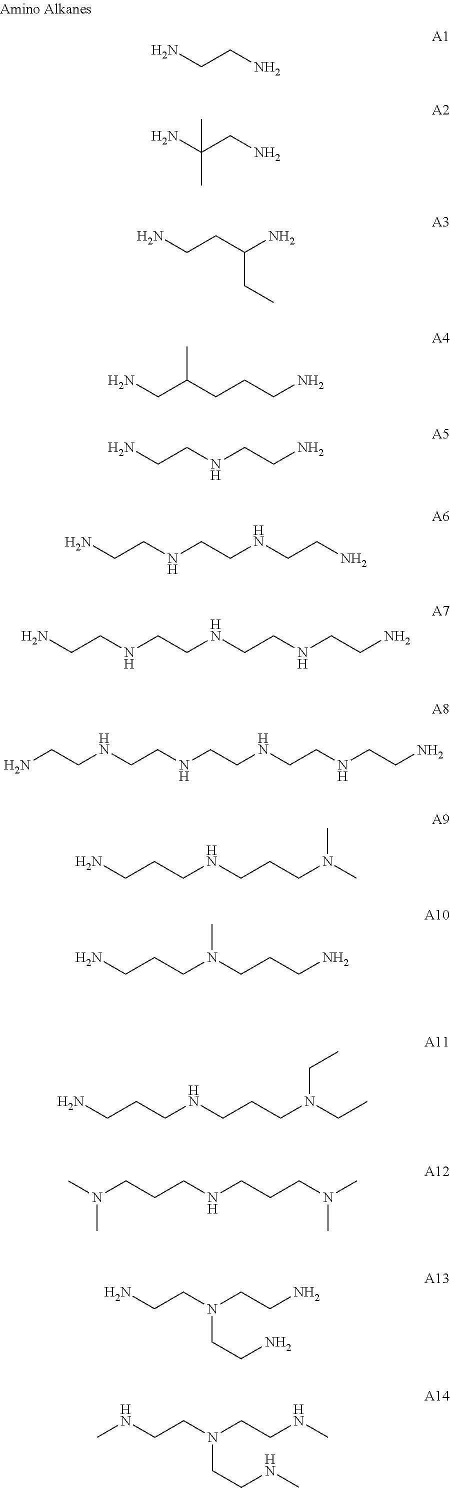

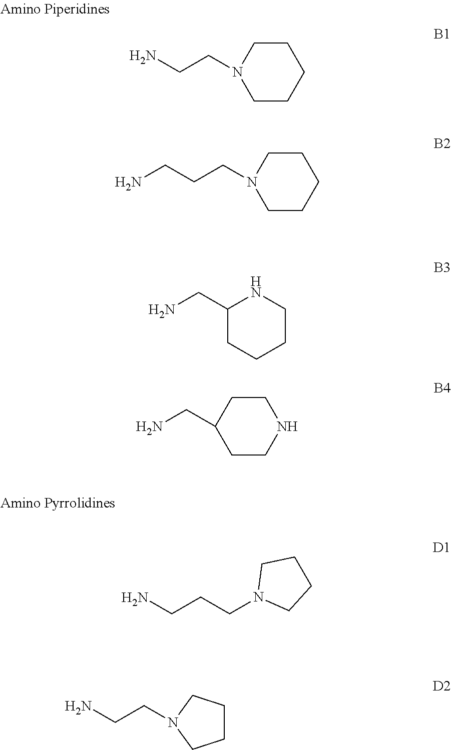

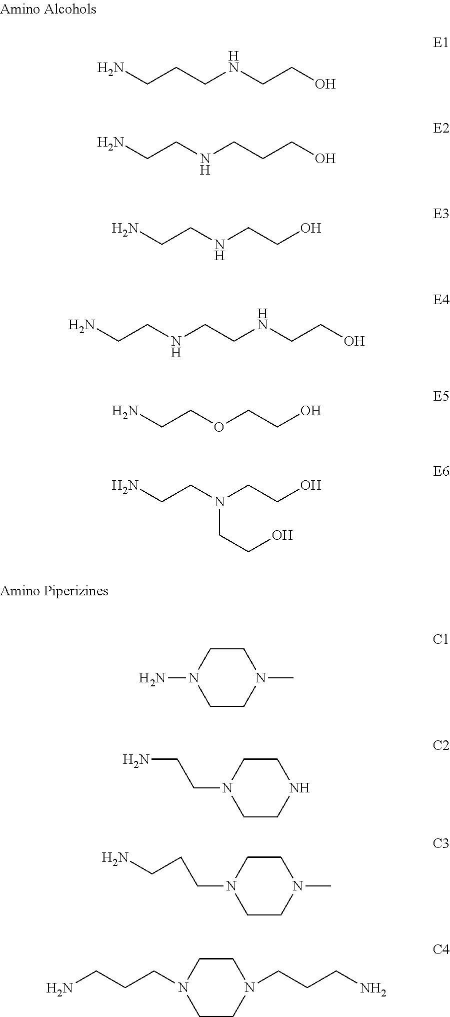

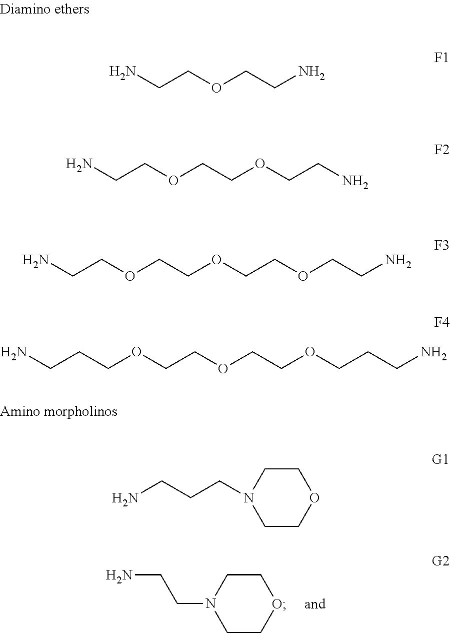 [SMILES: NCCOCCN ; NCCOCCOCCN ; NCCOCCOCCOCCN ; NCCCOCCOCCOCCCN ; NCCCN1CCOCC1 ; NCCN1CCOCC1]

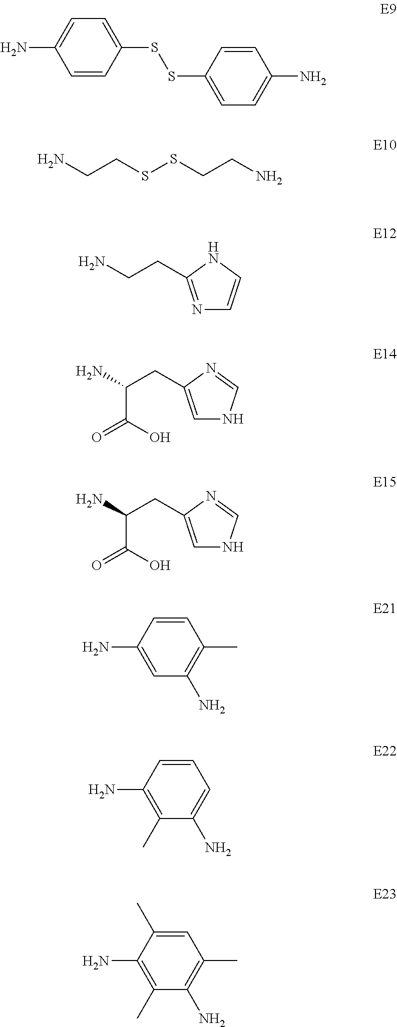

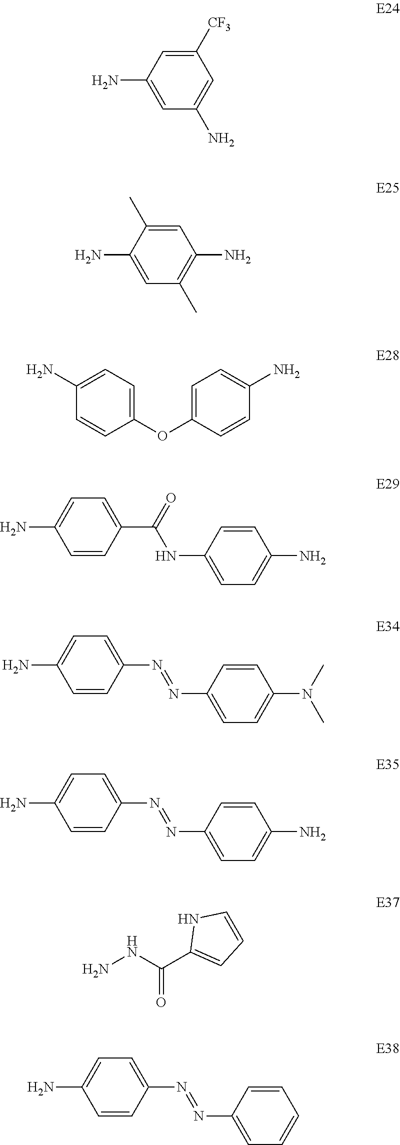

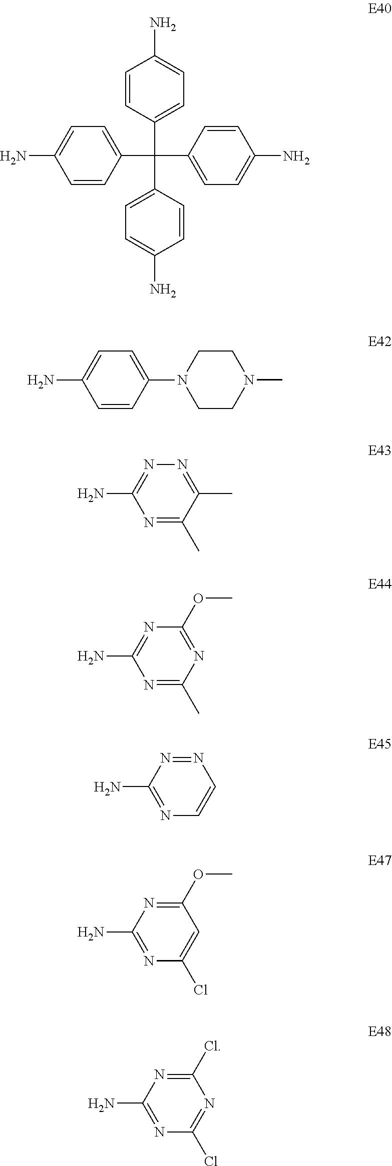

In certain aspects, —(CH₂)ₘ₁—(C═C)_{q}—(CH₂)ₘ₂—CH₃ is selected from:

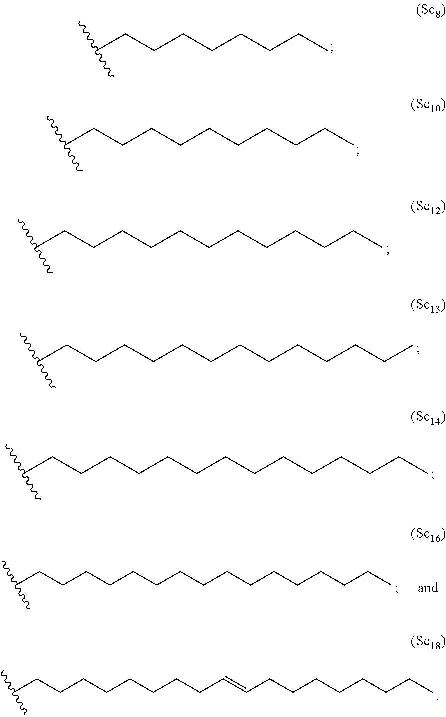

In certain aspects, the gene-editing protein is selected from the group consisting of CRISPR-associated nuclease, Cre recombinase, Flp recombinase, a meganuclease, a Transcription Activator-Like Effector Nuclease (TALEN), a Zinc-Finger Nuclease (ZFN), or a natural or engineered variant, family-member, orthologue, fragment or fusion construct thereof.

In certain aspects, the gene-editing protein comprises a Cas9 endonuclease.

In certain aspects, the nanoparticle further comprises a gRNA or DNA encoding a gRNA.

In certain aspects, the Cas9 endonuclease and the gRNA are encoded on the same plasmid. In other aspects, the Cas9 endonuclease and the gRNA are encoded on different plasmids.

In certain aspects, the nanoparticle comprises an mRNA molecule. In such aspects, the nanoparticle comprises a polymer-to-mRNA weight-to-weight ratios having a range from about 100:1 to about 5:1. In particular aspects, the polymer-to-mRNA weight-to-weight ratio is selected from 60:1, 50:1, 40:1, 30:1, and 20:1.

In particular aspects, the polymer of formula (I) has the following chemical structure having terminal amine end groups:

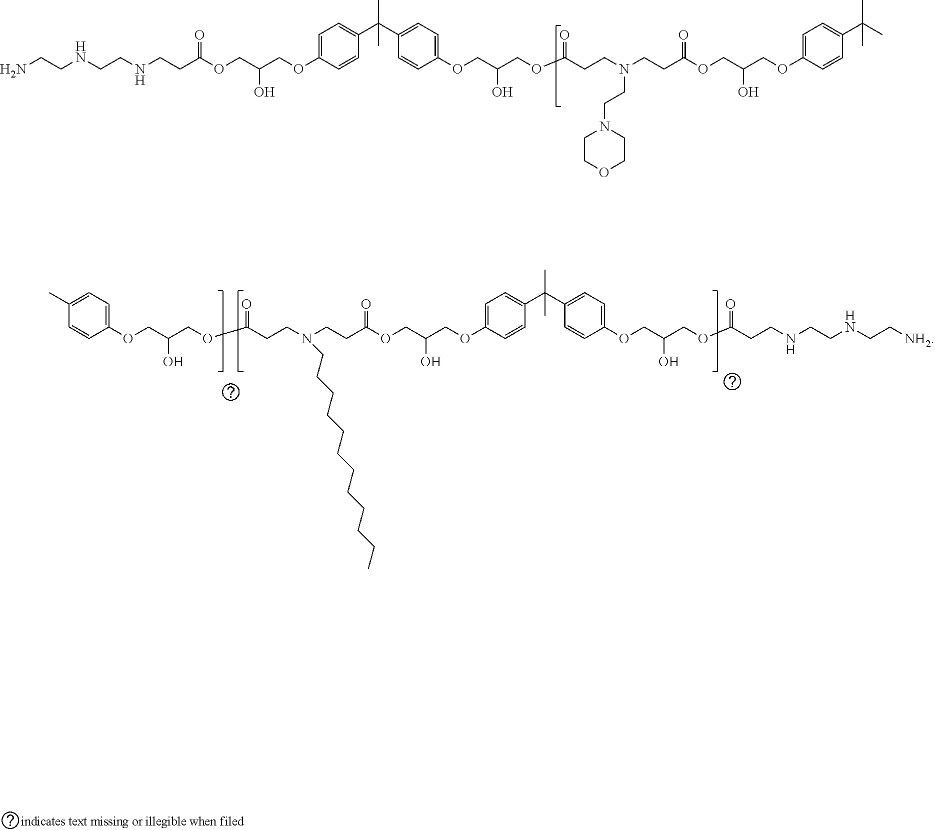

In more particular aspects, the terminal amine end groups of the polymer of formula (I) are replaced by a monomer selected from E1, E39, and E58.

In certain aspects, n and m are each independently an integer having a range from 1 to 10,000, 1 to 1,000, 1 to 100, 1 to 30, 1 to 20, 1 to 15, and 1 to 10, 1 to 5, 1 to 4, 1 to 3, 1 to 2, and 1.

In certain aspects, the nanoparticle has a size between about 100 nm to about 150 nm in diameter.

In certain aspects, the nanoparticle further comprises a poly(ethylene glycol) (PEG)-lipid.

In certain aspects, the nanoparticle further comprises an antibody targeting one or more molecules expressed on lung epithelial cells.

In other aspects, the presently disclosed subject matter provides a method for gene editing, comprising contacting a cell with the nanoparticle as disclosed hereinabove.

In certain aspects, the gene-editing protein directs site-specific target DNA disruption, mutation, deletion, or repair.

In certain aspects, the nanoparticle and cell are contacted in vivo. In other aspects, the nanoparticle and cell are contacted ex vivo.

In certain aspects, the cell is a mammalian cell. In particular aspects, the mammalian cell is a human cell.

In other aspects, the presently disclosed subject matter provides a method of treating a genetic disease, condition, or disorder, the method comprising administering a therapeutically effective amount of a nanoparticle disclosed hereinabove to a subject in need of treatment thereof.

In certain aspects, the disease, condition, or disorder comprises cystic fibrosis (CF). In particular aspects, the subject being treated for cystic fibrosis has a canonical splice site variant. In certain aspects, the canonical splice site variant introduces a premature stop codon (PTC). In certain aspects, the canonical splice variant comprises an alterations of the 5′GT splice motif or 3′AG splice motif. In particular aspects, the canonical splice site variant comprises c.2988+1G>A. In more particular aspects, the treatment corrects c.2988+1G>A in airway epithelia of the subject. In certain aspects, the subject is of African descent.

In certain aspects, the method further comprises administering a protein-targeted modulator therapy.

In certain aspects, the method comprises a systemic administration of the nanoparticle. In particular aspects, the systemic administration is intravenous (IV) or intranasal (IN).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows short circuit values for CFBE 3120+1 cells after transfection with nanoparticles and an adenine base editor (ABE) with guide RNA (mean±SEM);

FIG. 2A and FIG. 2B show representative reaction schemes for preparing the presently disclosed poly(β-amino esters (PBAEs), a generic chemical structure of a representative PBAE polymer, and representative monomers for preparing the presently disclosed PBAEs;

FIG. 3 shows in vitro transfection of CF8 3120+1 stable cells;

FIG. 4 shows in vitro transfection of primary nasal or bronchial epithelial cells;

FIG. 5 shows in vitro editing of CF8 3120+1 stable cells;

FIG. 6 shows in vitro editing of CF8 3120+1 stable cells. Short-circuit tracings show restoration of CFTR function in transfected cells, indicating successful editing;

FIG. 7 shows in vitro editing of CF8 3120+1 stable cells. In combination with ivacaftor, edited cells achieved approximately 12 μAmp current gain, with about 8% to about 10% WT restoration;

FIG. 8 demonstrates that different PBAE structures can efficiently transfect various organs after systemic injection;

FIG. 9A and FIG. 9B shows in vivo lung transfection via intranasal (IN) administration. FIG. 9A demonstrates that IN administration provides direct access to lungs, is non-invasive and easy to administer, and is unlikely to reach tissues outside the lungs. FIG. 9B demonstrates that IN administration of nanoparticles leads to strong expression of luciferase reporter gene in the lungs;

FIG. 10 demonstrates in vivo transfection of specific cells;

FIG. 11 show transfection of lung epithelial cells: CD45−CD31−CD326+;

FIG. 12 shows transfection of lung bronchial epithelial cells: CD45−CD31−CD326+CD24+;

FIG. 13 shows transfection of lung endothelial cells: CD45−CD31+;

FIG. 14 shows transfection of lung stromal cells: CD45−CD31−CD326−;

FIG. 15 shows transfection of lung leukocytes: CD45+;

FIG. 16A shows intranasal administration: APC transfection;

FIG. 16B demonstrates that systemic administration of NPs successfully edits not only epithelial cells of the lungs, but also other cell types in other organs, including liver (CD45−, CD31−), liver (CD45+, CD11b+), spleen (CD45+, CD3+), and liver (CD45+, CD19+);

FIG. 17 demonstrates in vivo transfection of lung cells. PBAE nanoparticles transfect endothelial cells and bronchial epithelial cells in the lungs after IV administration. Multiple administrations were well tolerated and improved transfection efficiency;

FIG. 18 shows flow cytometry in Ai9 mice after systemic delivery of 5 μg Cre mRNA with or without a targeting ligand (CD326) and number of injections (1×, 3×, 9×) displaying relevant tdTomato+lung cell types;

FIG. 19A and FIG. 19B show (FIG. 19A) Percent increase in CFTR+ cells between unedited and edited samples. (FIG. 19B) Comparison of percent CFTR+ cells in cell type clusters between unedited and edited samples;

FIG. 20A and FIG. 20B show percent transfection in (FIG. 20A) CFBE cells and (FIG. 20B) primary HBE cells for a suboptimized polymer set (w/w=40) with error bars representing N=4 with mean±SEM;

FIG. 21 shows short circuit values for CFBE 3120+1 cells after transfection with nanoparticles and ABE with guide RNA (mean±SEM, N=3);

FIG. 22 is a diagram showing a primary human primary nasal (HNE) and human bronchial epithelial (HBE) nanoparticle screen with flow cytometry;

FIG. 23 shows representative polymers used in the screen described in FIG. 22;

FIG. 24A, FIG. 24B, FIG. 24C, and FIG. 24D show the flow cytometry gating breakdown;

FIG. 25A shows the normalized cell count of CF genotype (top) and wild type (bottom) HBE (left) and HNE (right) cells treated with the polymers shown in FIG. 23 at various polymer concentrations;

FIG. 25B shows the % GFP transfection of CF genotype (top) and wild type (bottom) HBE (left) and HNE (right) cells treated with the polymers shown in FIG. 23 at various polymer concentrations;

FIG. 25C shows the normalized MFI of CF genotype (top) and wild type (bottom) HBE (left) and HNE (right) cells treated with the polymers shown in FIG. 23 at various polymer concentrations;

FIG. 26 shows C57BL/6 in vivo lung expression with polymers having different endcaps E63, E58, E1, and E39;

FIG. 27A and FIG. 27B show the dissociated cell populations using different dissociation protocols (FIG. 27A) and the total cell count for different dissociation protocols (FIG. 27B);

FIG. 28A and FIG. 28B are an Ai9 mouse study, N=6; 5 μg NPs injected 3×; N=3 run on flow cytometry with the Dispase lung dissociation protocol (gating below); and N=3 for histology/IHC. Data shown in FIG. 28B;

FIG. 29A, FIG. 29B, and FIG. 29C demonstrate that the Dispase method shows similar and more consistent results compared to previous 3× dosing;

FIG. 30A and FIG. 30B show Ai9 mouse data at dosages of 3×, 3×+CD326, 6×, and 6×+CD326; and

FIG. 31A and FIG. 31B show the percent transfection of Ai9 tdTomato+lung cells in various treatment groups (FIG. 31A) and % tdTomato positive cells in bronchial cells in various treatment groups (FIG. 31B).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Polymer-Nucleic Acid Nanoparticles for Gene Editing

In some embodiments, the presently disclosed subject matter provides nanoparticles less than 1000 nm in diameter comprising a biodegradable synthetic polymer, one or more nucleic acids, and a stabilizing agent, such as PEGylated lipid. In contrast to previously described nanoparticles that largely target the liver or phagocytic cells, the presently disclosed nanoparticles can be used to deliver genes to the lung epithelium and other tissues for gene editing. This technology can be used to correct genetic mutations in diseases, such as cystic fibrosis.

More particularly, the presently disclosed nanoparticles comprise poly(β-amino esters (PBAEs) having ester linkages (—C(═O)—O), which allow for quick hydrolytic degradation and low toxicity; secondary/tertiary amines, which can be reversibly protonated, positively charged, and can electrostatically complex with negatively charged nucleic acids; and a lipophilic side-chain, which enables tight NP complexation, hydrophobic interactions with other sidechains and other lipophilic materials, and interactions with cells.

Advantages of PBAEs for gene editing include, but are not limited to, PBAEs can form nanoparticles with nucleic acids; a combinatory library can be screened in vitro to find polymers having a high transfection efficacy; Cas9 or other editors and sgRNA can be encoded in DNA or mRNA; PBAE NPs can be used for gene editing in vivo; and ABE delivered to CF epithelial cells leads to gain of function.

Further advantages of the presently disclosed polymers for gene therapy include, but are not limited to, safety, for example, a minimal immune response, minimal cellular toxicity, and non-carcinogenic; large cargo capacity; flexibility to target different cells and tissues; low resistance to repeated administration; and ease of production and quality control.

### A. Poly(β-Amino Esters for Gene Editing

As used herein, “biodegradable” polymers and/or nanoparticles are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). Such components preferably do not induce inflammation or other adverse effects in vivo. In some instances, the chemical reactions relied upon to break down the biodegradable compounds are uncatalyzed.

In certain embodiments, the biodegradable polymers and/or nanoparticles comprise a chemical moiety having one or more degradable linkages, such as an ester linkage, a disulfide linkage, an amide linkage, an anhydride linkage, and a linkage susceptible to enzymatic degradation. Representative degradable linkages include, but are not limited to:

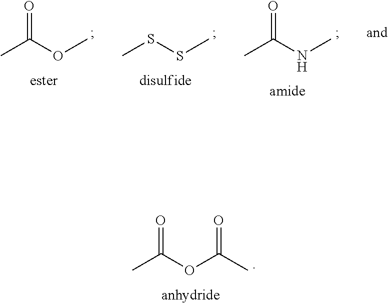

In some embodiments, the biodegradable polymer and/or nanoparticle comprises a poly(beta-amino ester) (PBAE). Exemplary PBAEs suitable for use with the presently disclosed subject matter include those disclosed in:
U.S. Pat. No. 9,884,118 for Multicomponent Degradable Cationic Polymers, to Green et al., issued Feb. 6, 2018;
U.S. Pat. No. 9,802,984 for Biomimetic Peptide and Biodegradable Delivery Platform for the Treatment of Angiogenesis- and Lymphangiogenesis-Dependent Diseases, to Popel et al., issued Oct. 31, 2017;
U.S. Pat. No. 9,717,694 for Peptide/Particle Delivery Systems, to Green et al., issued Aug. 1, 2017;
U.S. Pat. No. 8,992,991 for Multicomponent Degradable Cationic Polymers, to Green et al., issued Mar. 31, 2015;
U.S. Patent Application Publication No. 20180256745 for Biomimetic Artificial Cells: Anisotropic Supported Lipid Bilayers on Biodegradable Micro and Nanoparticles for Spatially Dynamic Surface Biomolecule Presentation, to Meyer et al., published Sep. 13, 2018;
U.S. Patent Application Publication No. 20180112038 for Poly(Beta-Amino Ester)-Co-Polyethylene Glycol (PEG-PBAE-PEG) Polymers for Gene and Drug Delivery, to Green et al., published Apr. 26, 2018;
U.S. Patent Application Publication No. 20170216363 for Nanoparticle Modification of Human Adipose-Derived Mesenchymal Stem Cells for Treating Brain Cancer and other Neurological Diseases, to Quinones-Hinojosa and Green, published Aug. 3, 2017;
U.S. Patent Application Publication No. 20150273071 for Bioreducible Poly (Beta-Amino Ester)s For siRNA Delivery, to Green et al., published Oct. 1, 2015;
U.S. Pat. No. 8,287,849 for Biodegradable Poly(beta-amino esters) and Uses Thereof, to Langer, et al., issued Oct. 16, 2012;
International PCT Patent Application Publication No. WO2020198145 for Gene Delivery Particles to Induce Tumor-Derived Antigen Presenting Cells, to Green, published Oct. 1, 2020;
International PCT Patent Application Publication No. WO2020077159 for Poly(Beta-Amino Ester) Nanoparticles for the Non-Viral Delivery of Plasmid DNA for Gene Editing and Retinal Gene Therapy, to Green et al., published Apr. 16, 2020;
each of which is incorporated by reference in their entirety.

Generally, the presently disclosed multicomponent degradable cationic polymers include a backbone derived from a diacrylate monomer (designated herein below as “B”), an amino-alcohol hydrophilic side-chain monomer (designated herein below as “S”), a hydrophobic side-chain monomer, and an amine-containing endcapping monomer (designated herein below as “E”). The endcapping group structures are distinct and separate from the polymer backbone structures and the side chain structures of the intermediate precursor molecule for a given polymeric material.

The presently disclosed PBAE compositions can be designated, for example, as B5-S4-E7 or 547, in which R is B5, R′ is S4, and R″ is E7, and the like, where B is the backbone and S is the side chain, followed by the number of carbons in their hydrocarbon chain, e.g., S4 comprises 4 alkylene groups. Endcapping monomers, E, are sequentially numbered according to similarities in their amine structures. Further, in some embodiments, the presently disclosed PBAE includes a hydrophobic side-chain, which is designated SC-XX, with XX being the number of carbon atoms in the chain.

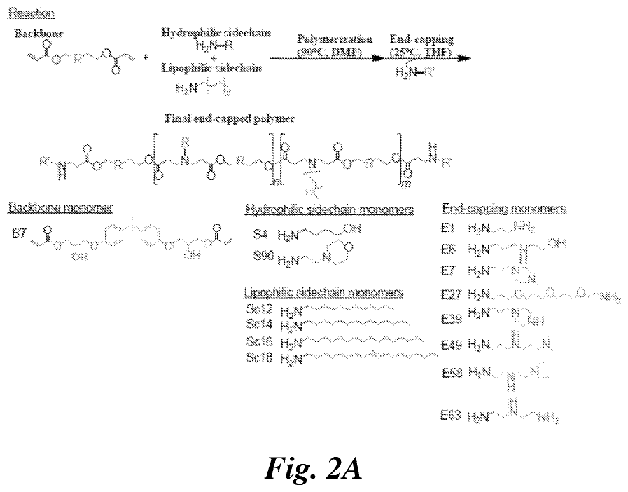

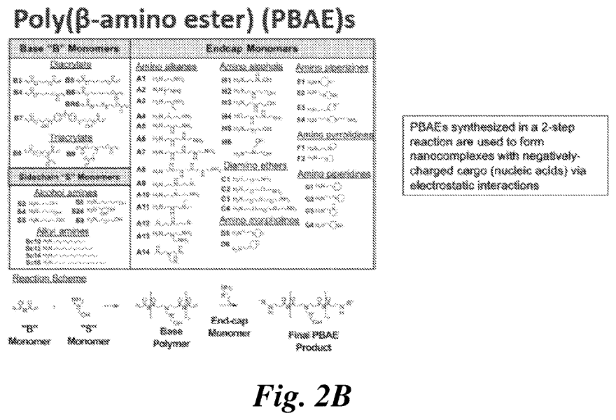

As shown in the reaction scheme provided in FIG. 2, acrylate monomers can be condensed with amine-containing side chain monomers. In some embodiments, the side chain monomers comprise a primary amine, but, in other embodiments, comprise secondary and tertiary amines. Side chain monomers may further comprise a C₁ to C₈ linear or branched alkylene, which is optionally substituted. Illustrative substituents include hydroxyl, alkyl, alkenyl, thiol, amine, carbonyl, and halogen.

Table 1 and Table 2 present, in more detail, particular monomers used for PBAE library synthesis. Acrylate terminated polymers were synthesized from small molecule diacrylate and primary amine monomers followed by high-throughput endcapping with 37 monomers organized into different structural categories. In certain embodiments, the linear and/or branched PBAE polymer has a molecular weight of from 5 to 10 kDa, or a molecular weight of from 10 to 15 kDa, or a molecular weight of from 15 to 25 kDa, or a molecular weight of from 25 to 50 kDa.

Accordingly, in some embodiments, the presently disclosed subject matter provides a nanoparticle comprising a compound of formula (I) and one or more nucleic acids:

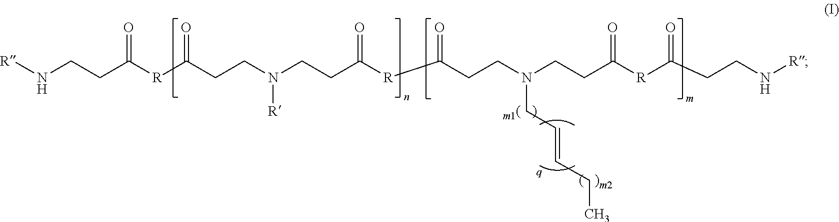

wherein: m and n are each independently an integer from 1 to 10,000; m1 is an integer selected from 0, 1, 2, 3, 4, 5, 6, 7, 8, 9, and 10; m2 is an integer selected from the group consisting of 1, 2, 3, 4, 5, 6, 7, 8, 9, 10, 11, 12, 13, 14, 15, 16, 17, 18, 19, and 20; q is an integer selected from 0 or 1; wherein —(CH₂)ₘ₁—(C═C)_{q}—(CH₂)ₘ₂—CH₃ comprises a hydrophobic sidechain; R comprises a divalent radical comprising a biodegradable ester linkage and/or a bioreducible disulfide linkage; R′ is hydrophilic sidechain comprising a monovalent radical derived from a hydrophilic amine monomer; R″ is monovalent radical derived from an amine-containing end capping group; and pharmaceutically acceptable salts thereof.

In certain embodiments, R is selected from the group consisting of:

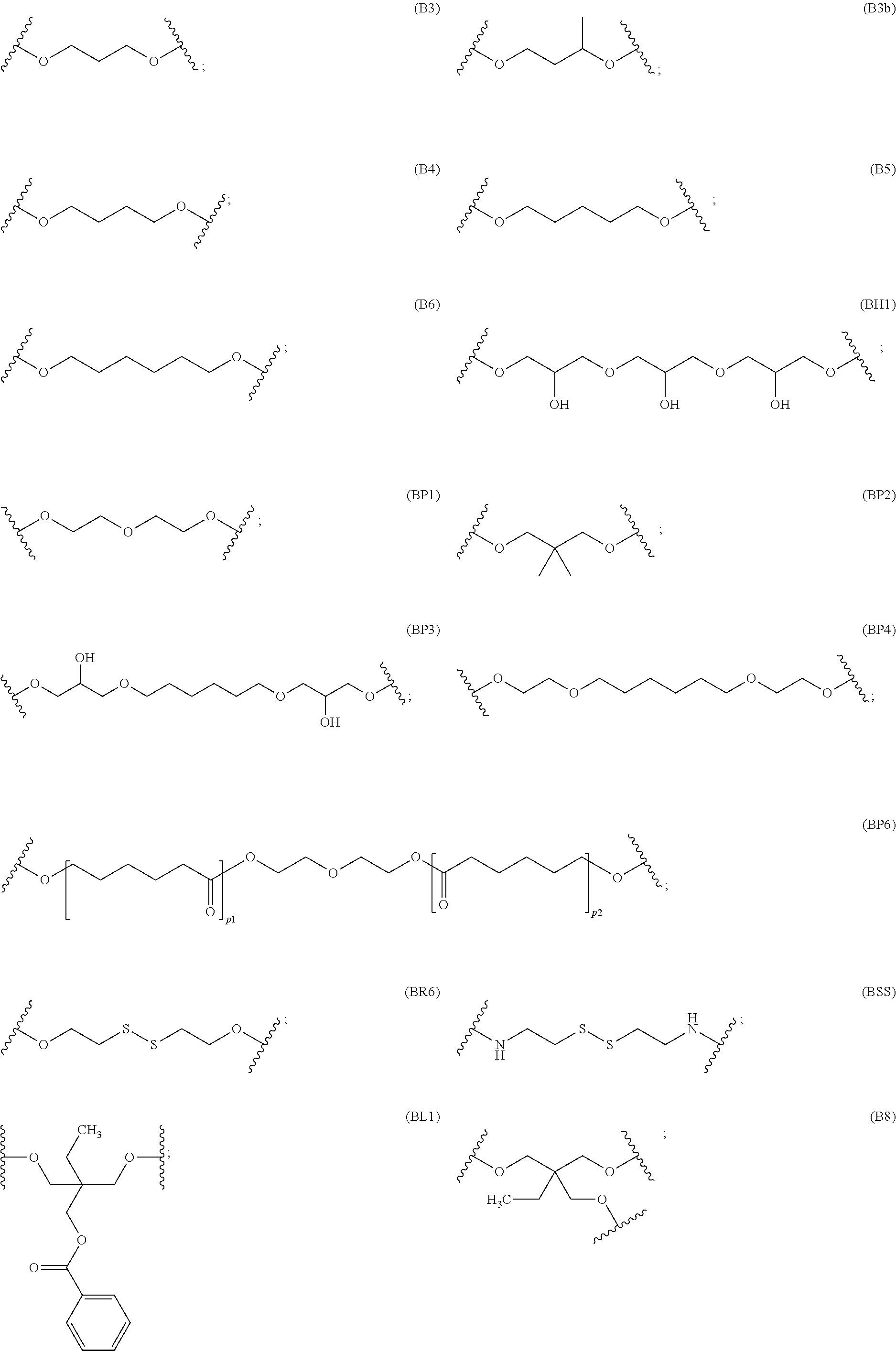

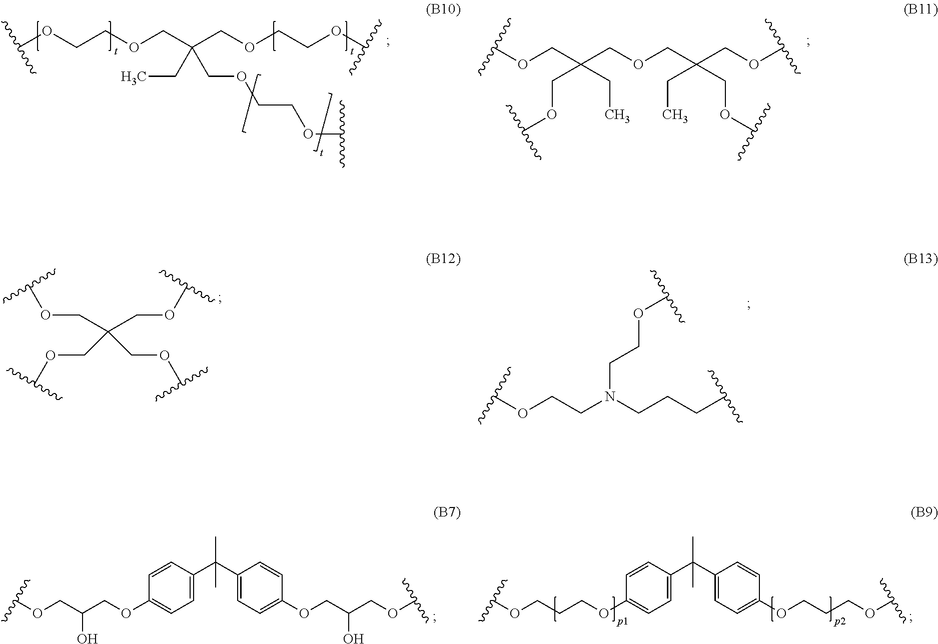

wherein each p1, p2, and t is independently an integer selected from the group consisting of 1, 2, 3, 4, 5, 6, 7, 8, 9, and 10.

In certain embodiments, R′ is selected from the group consisting of:

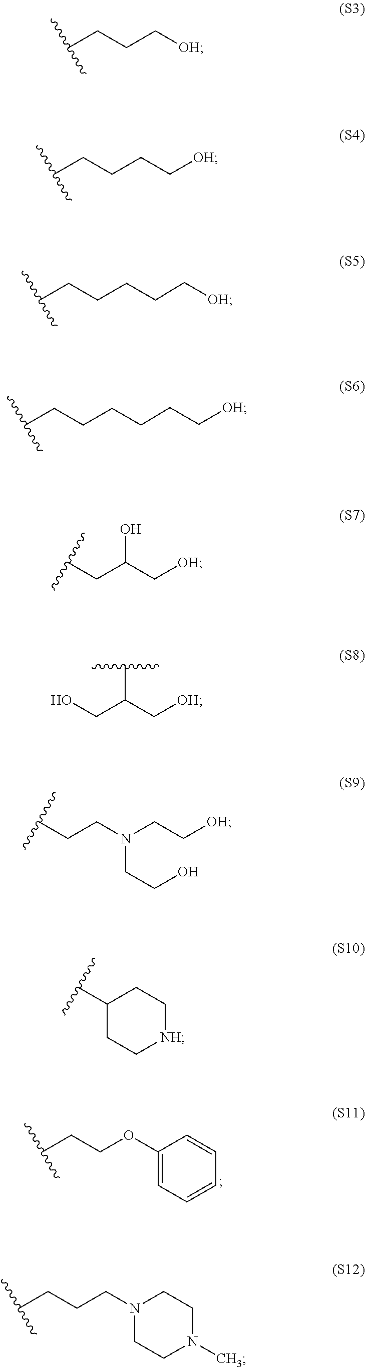

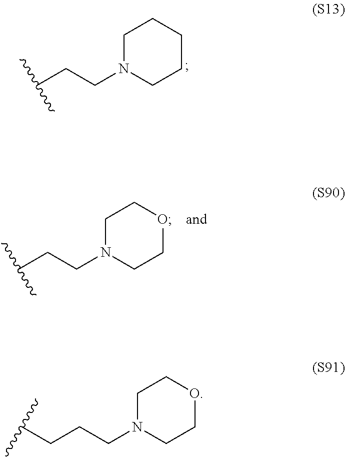 [SMILES: *CCN1CCCCC1 ; *CCN1CCOCC1 ; "and" ; *CCCN1CCOCC1]

In certain embodiments, R″ is selected from the group consisting of:

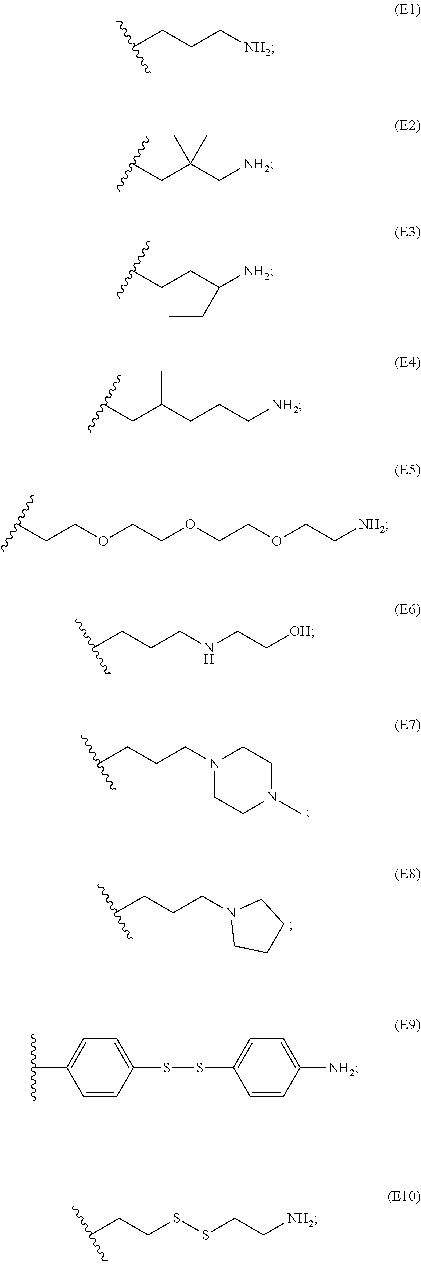

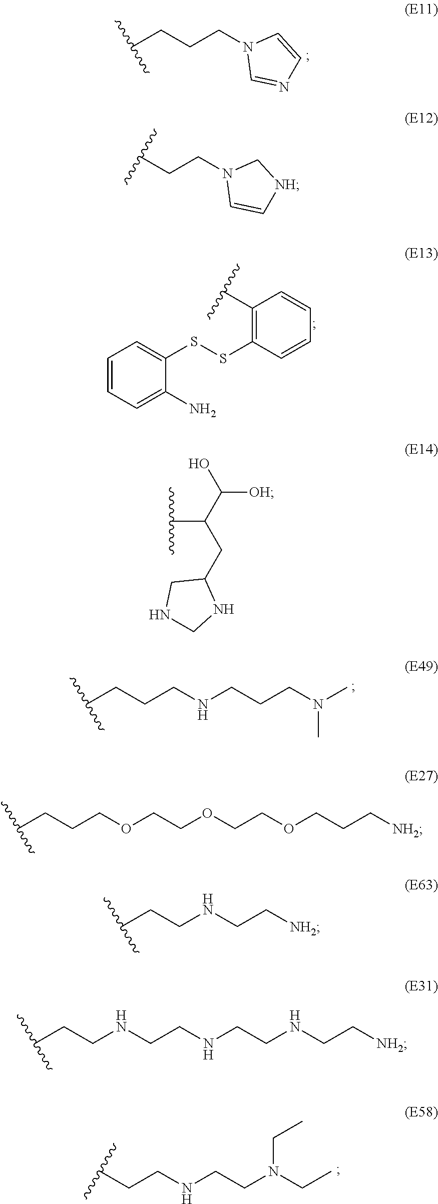

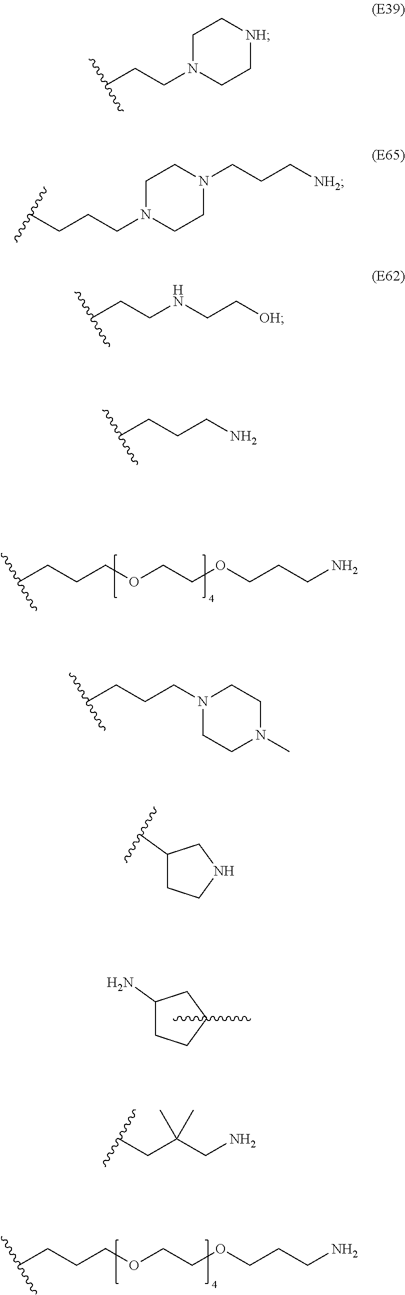

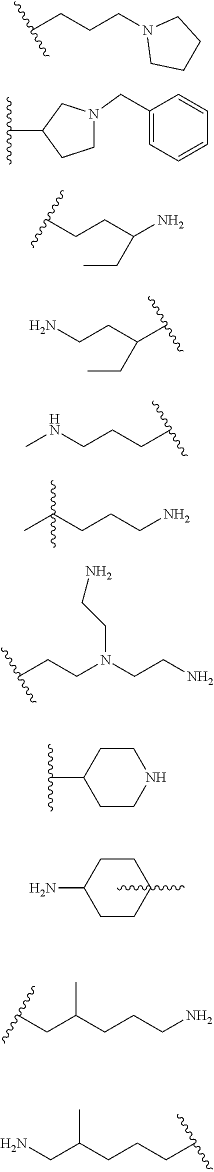

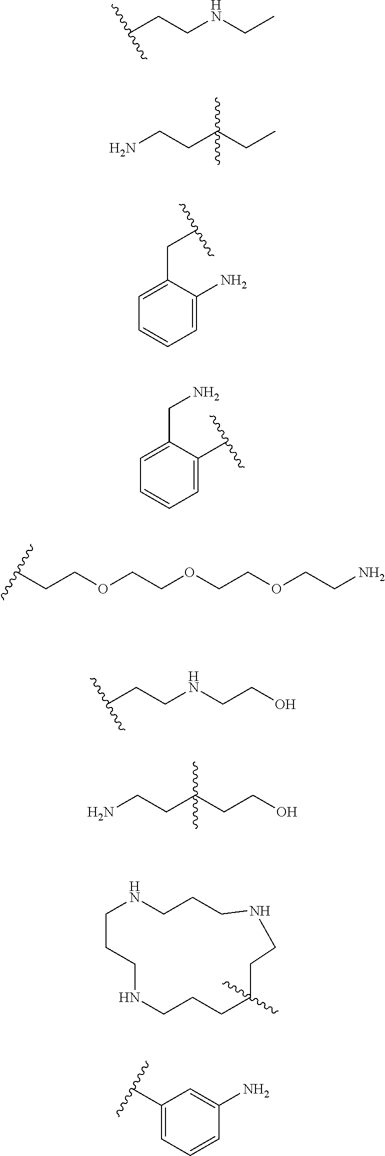

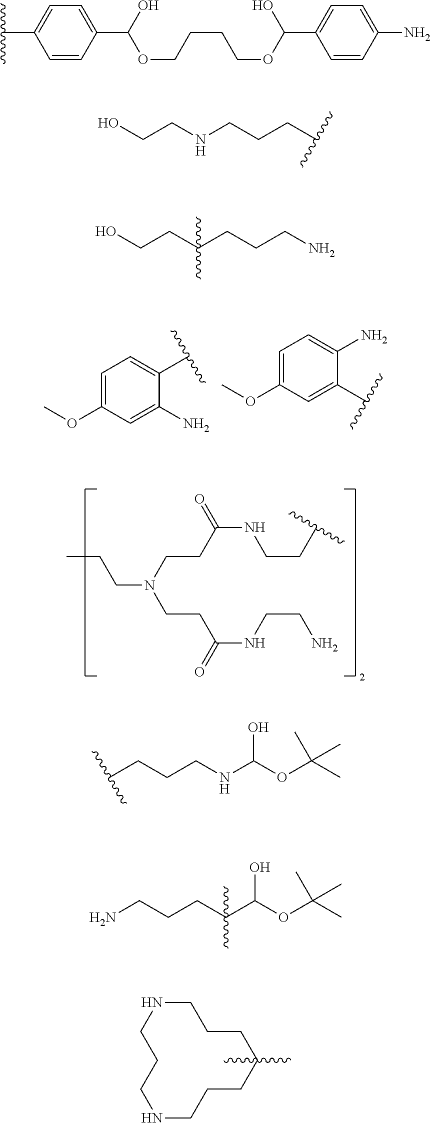

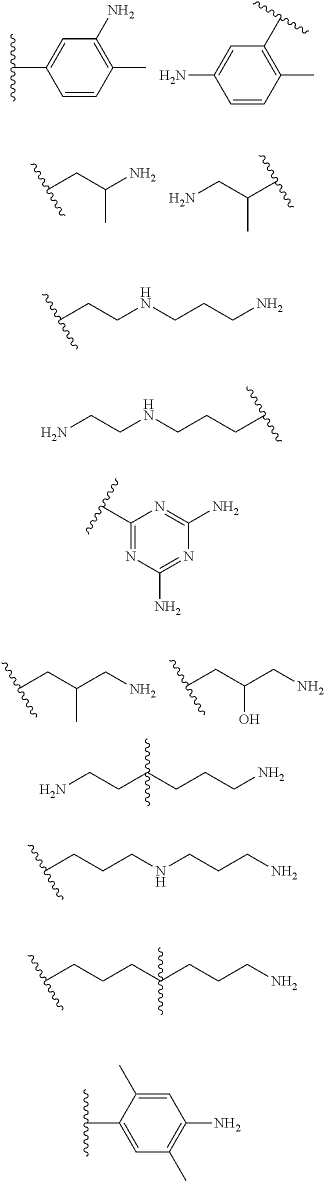

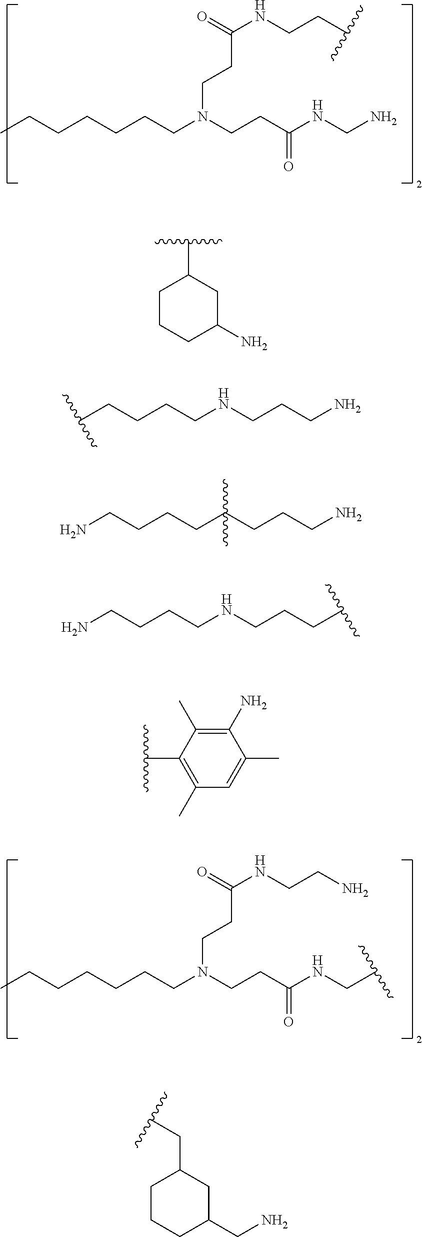

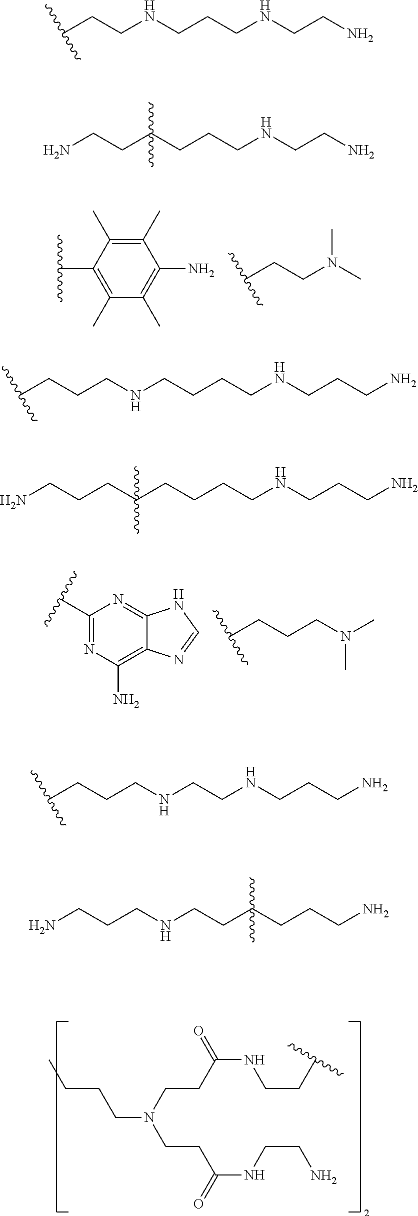

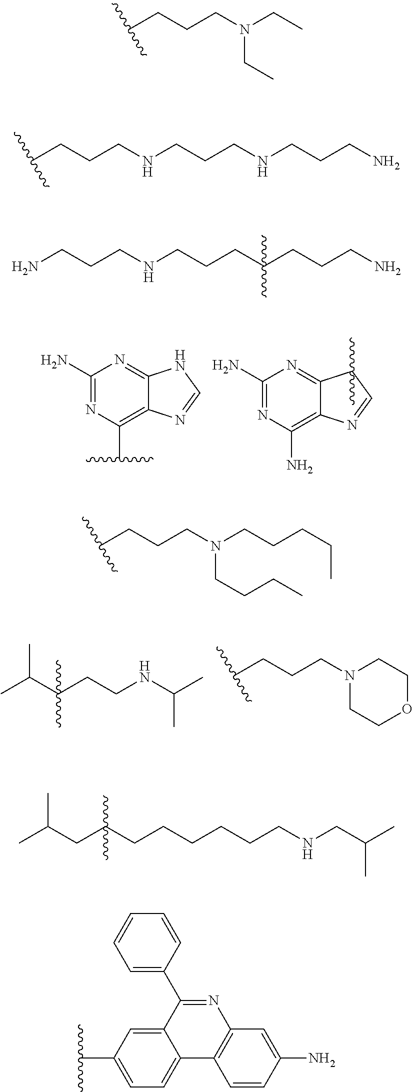

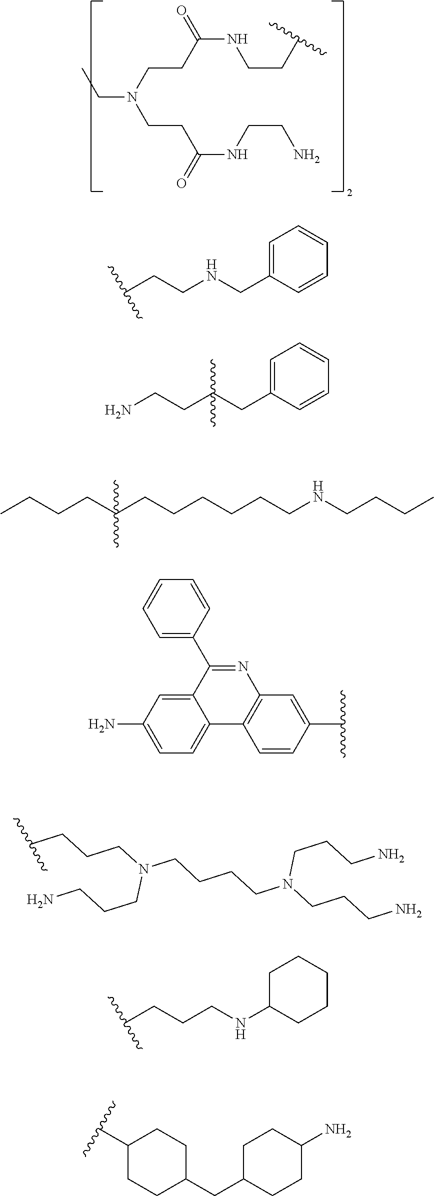

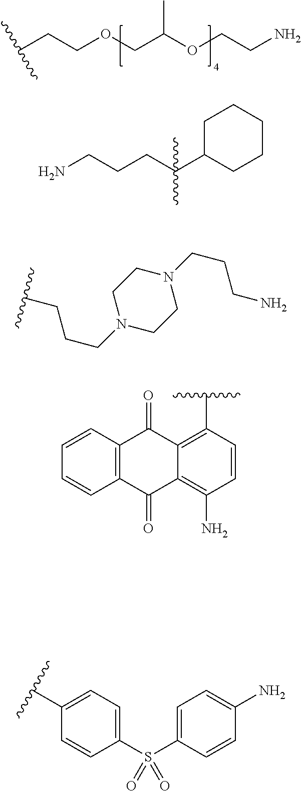

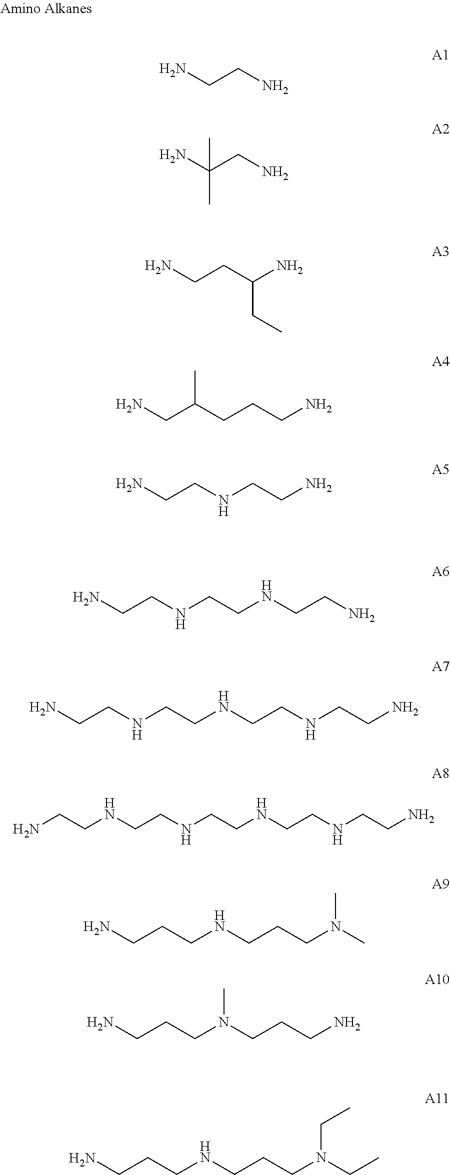

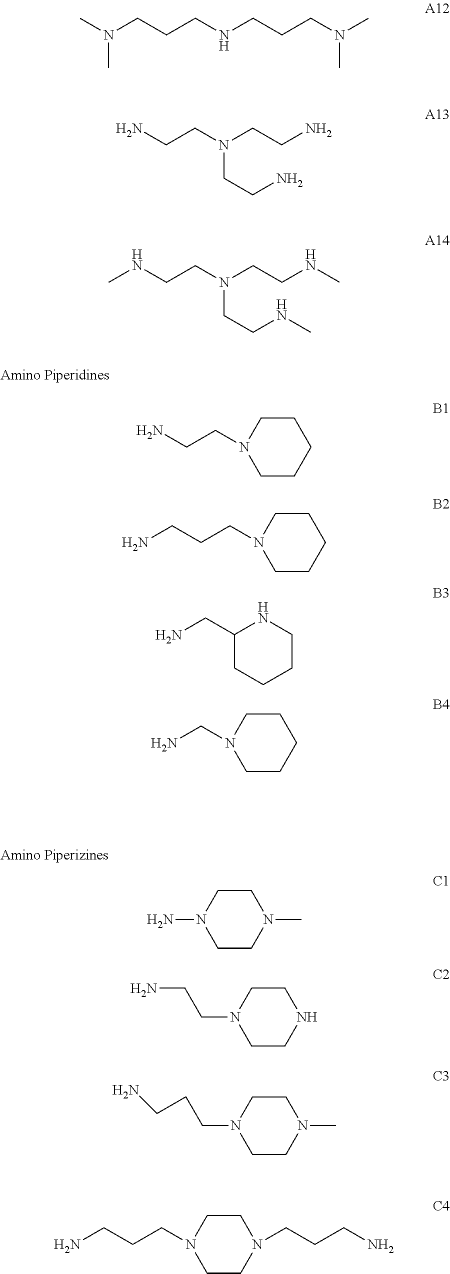

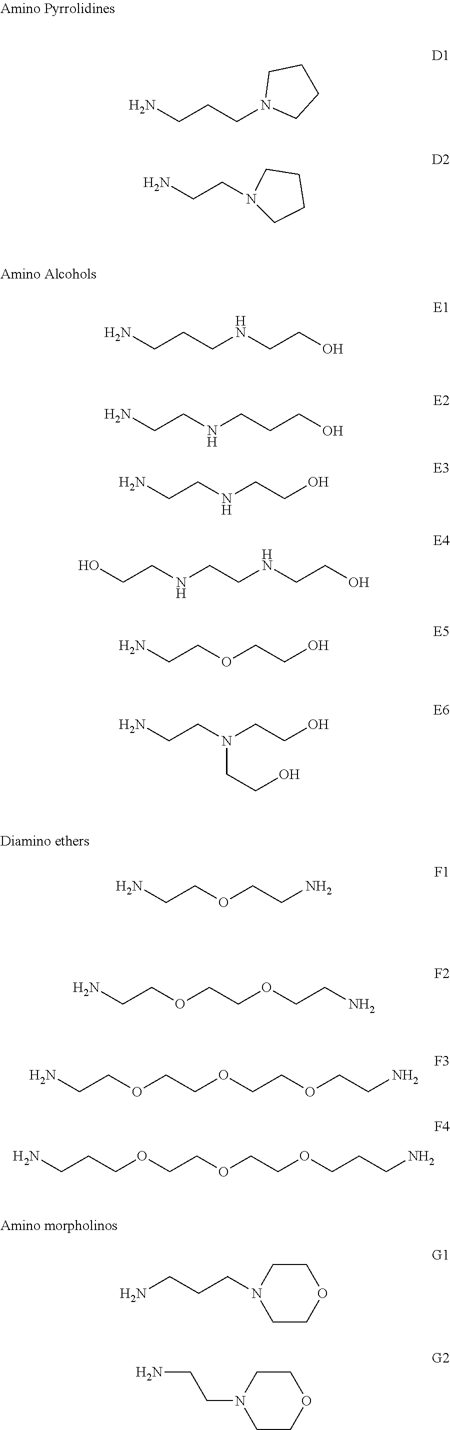

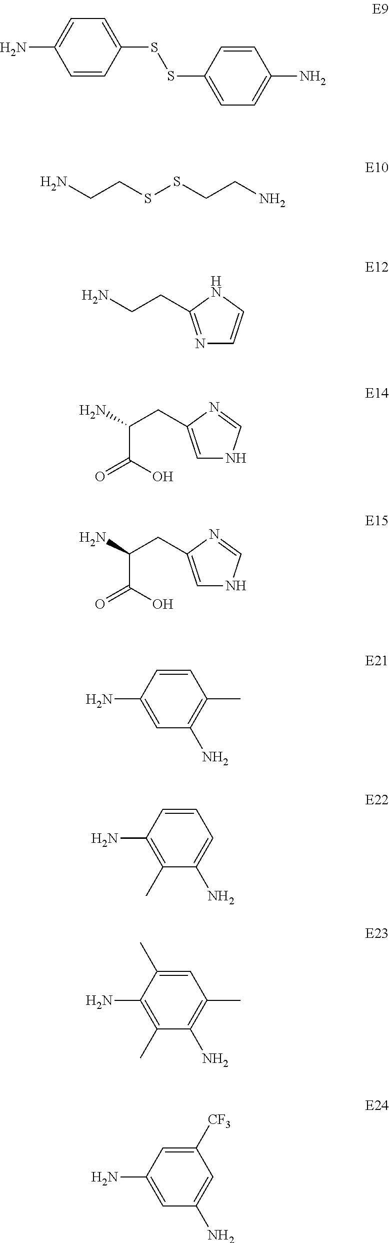

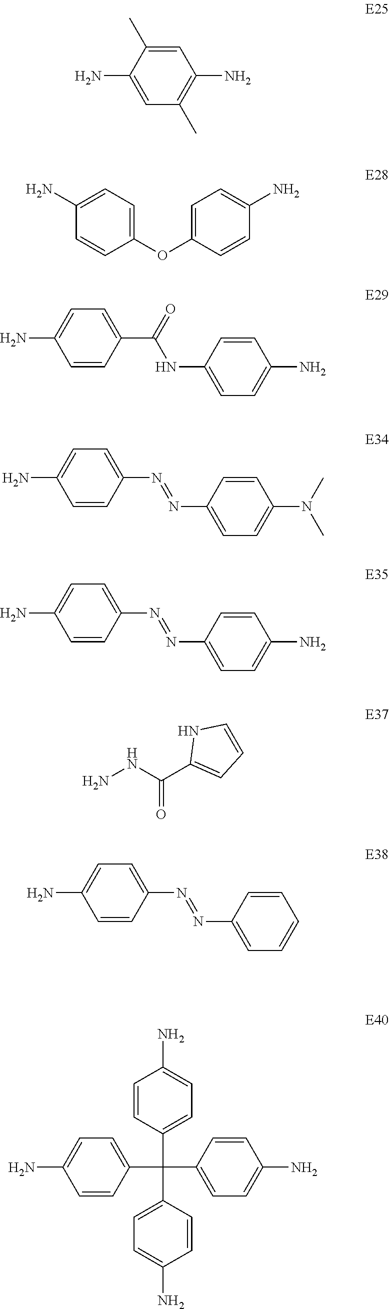

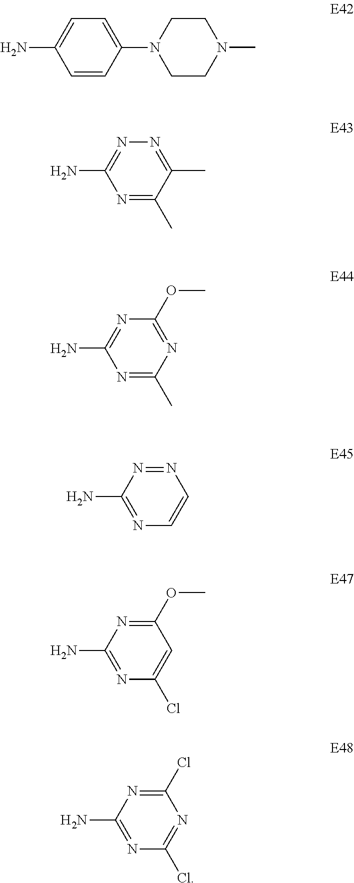

In certain embodiments, —(CH₂)ₘ₁—(C═C)_{q}—(CH₂)ₘ₂—CH₃ is selected from the group consisting of:

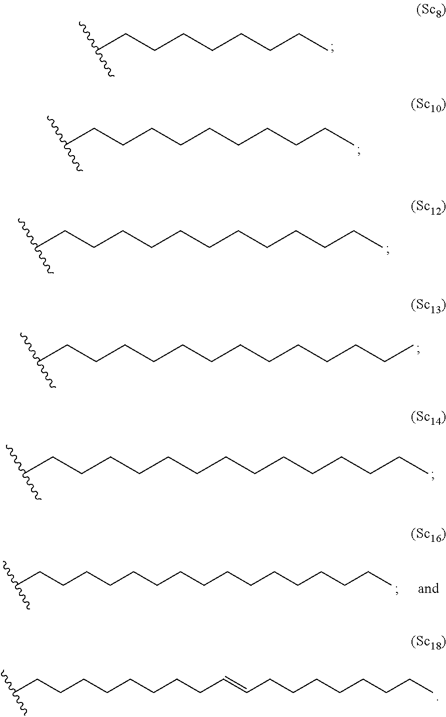

In particular embodiments, R is selected from the group consisting of:

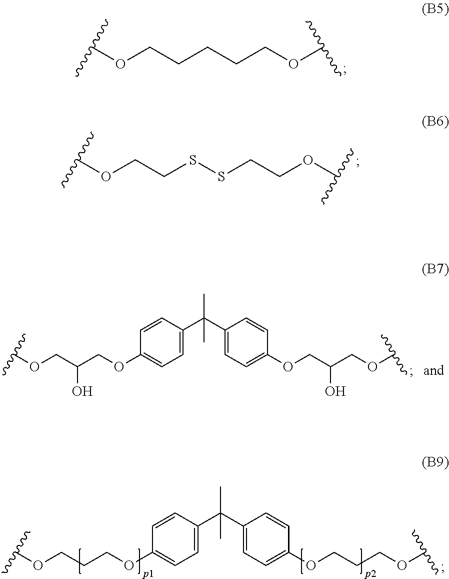

wherein each p1, p2, and t is independently an integer selected from the group consisting of 1, 2, 3, 4, 5, 6, 7, 8, 9, and 10.

In particular embodiments, R′ is selected from the group consisting of:

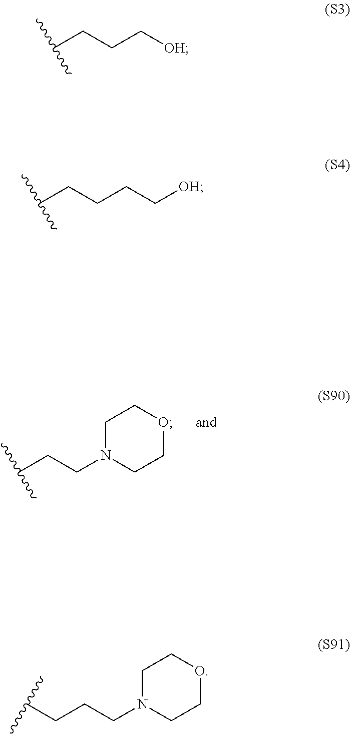 [SMILES: *CCCO ; *CCCCO ; *CCN1CCOCC1 ; "and" ; *CCCN1CCOCC1]

In particular embodiments, R″ is selected from the group consisting of:

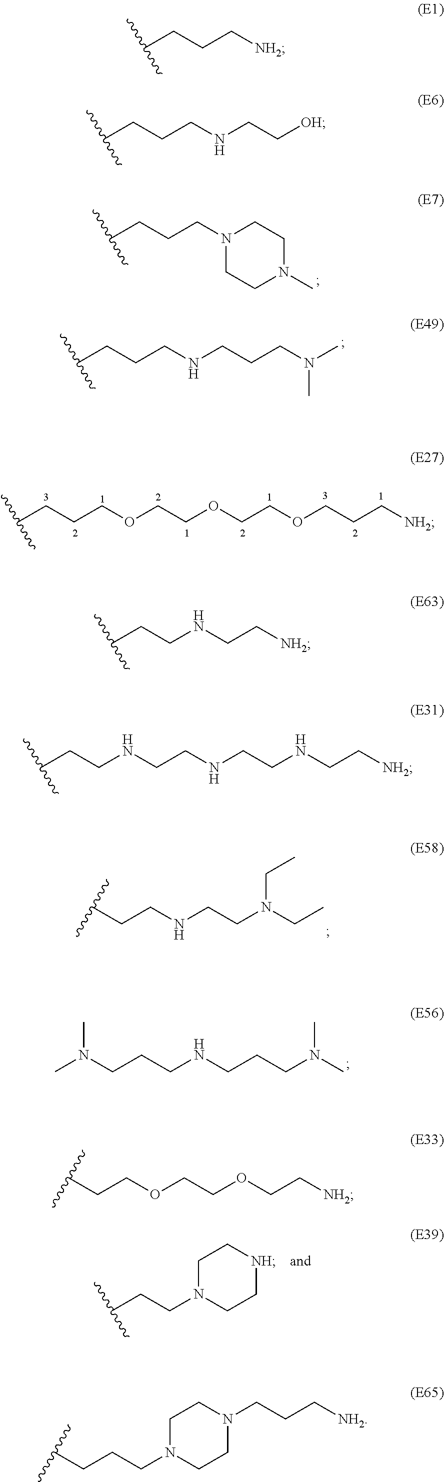

In particular embodiments, —(CH₂)ₘ₁—(C═C)_{q}—(CH₂)ₘ₂—CH₃ is selected from the group consisting of:

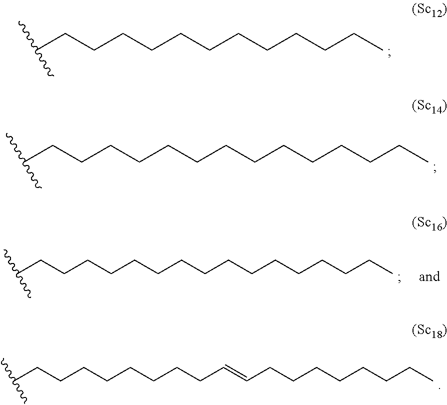 [SMILES: *CCCCCCCCCCCC ; *CCCCCCCCCCCCCC ; *CCCCCCCCCCCCCCCC ; *CCCCCCCC/C=C/CCCCCCCC]

In more particular embodiments, the compound of formula (I) comprises a combination of: (a) an R group selected from the group consisting of B5, B7, B9, and BR6; (b) an R′ group selected from the group consisting of S3, S4, S90, and S91; (c) an R″ group selected from the group consisting of E1, E6, E7, E27, E31, E33, E39, E49, E56, E58, E63, and E65; and (d) an —(CH₂)ₘ₁—(C═C)_{q}—(CH₂)ₘ₂—CH₃ moiety selected from the group consisting of Sc12, Sc14, Sc16, and Sc18.

In particular embodiments, R is:

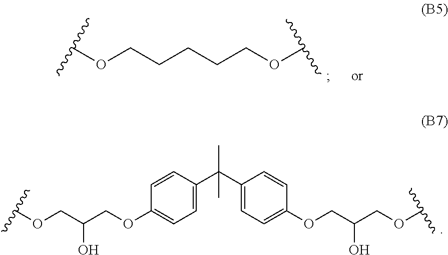 [SMILES: *OCCCCCO* ; *OCC(O)COc1ccc(C(C)(C)c2ccc(OCC(O)CO*)cc2)cc1]

In particular embodiments, R′ is:

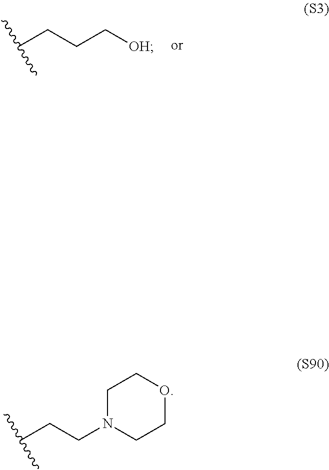 [SMILES: *CCCO ; "or" ; *CCN1CCOCC1]

In particular embodiments, R″ is:

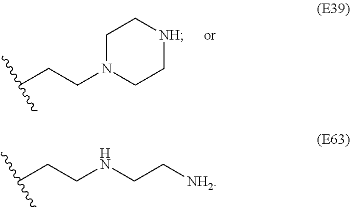 [SMILES: *CCN1CCNCC1 ; *CCNCCN]

In particular embodiments, —(CH₂)ₘ₁—(C═C)_{q}—(CH₂)ₘ₂—CH₃ is Sc12.

In certain embodiments, the gene-editing protein is selected from the group consisting of CRISPR-associated nuclease, Cre recombinase, Flp recombinase, a meganuclease, a Transcription Activator-Like Effector Nuclease (TALEN), a Zinc-Finger Nuclease (ZFN), or a natural or engineered variant, family-member, orthologue, fragment or fusion construct thereof.

In certain embodiments, the gene-editing protein comprises a Cas9 endonuclease.

In certain embodiments, the nanoparticle further comprises a gRNA or DNA encoding a gRNA.

In certain embodiments, the Cas9 endonuclease and the gRNA are encoded on the same plasmid. In other embodiments, the Cas9 endonuclease and the gRNA are encoded on different plasmids.

In certain embodiments, the nanoparticle comprises an mRNA molecule. In such embodiments, the nanoparticle comprises a polymer-to-mRNA weight-to-weight ratios having a range from about 100:1 to about 5:1. In particular embodiments, the polymer-to-mRNA weight-to-weight ratio is selected from 60:1, 50:1, 40:1, 30:1, and 20:1.

In particular embodiments, the polymer of formula (I) has the following chemical structure having terminal amine end groups:

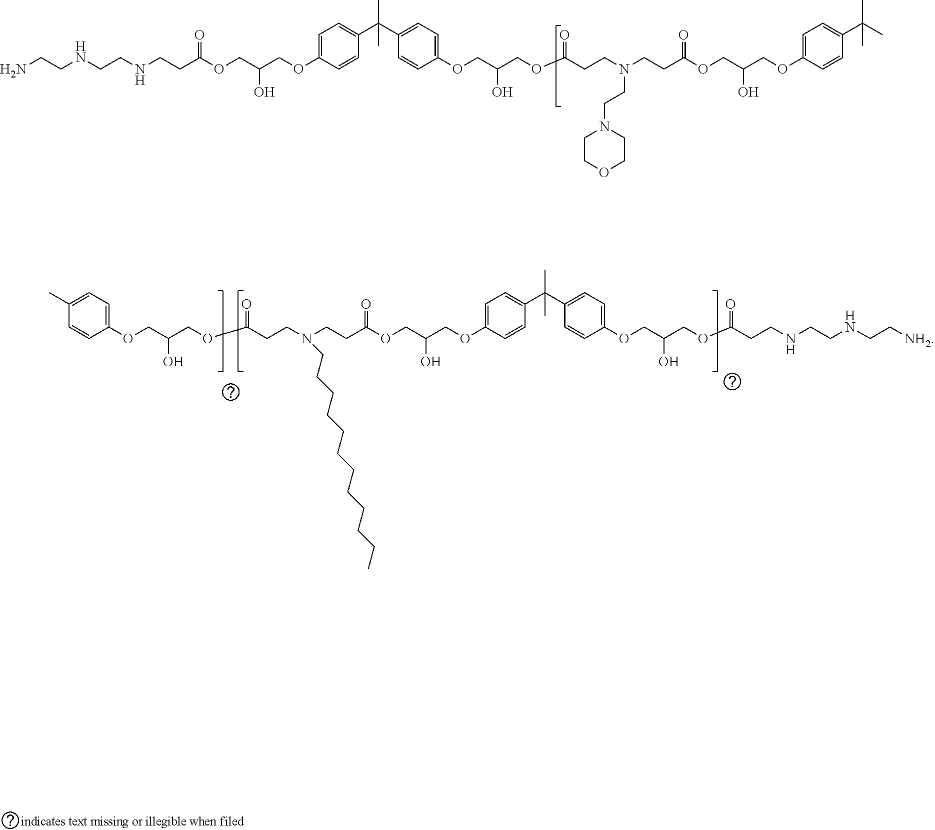

In more particular embodiments, the terminal amine end groups of the polymer of formula (I) are replaced by a monomer selected from E1, E39, and E58.

In certain embodiments, n and m are each independently an integer having a range from 1 to 10,000, 1 to 1,000, 1 to 100, 1 to 30, 1 to 20, 1 to 15, and 1 to 10, 1 to 5, 1 to 4, 1 to 3, 1 to 2, and 1.

In certain embodiments, the nanoparticle further comprises an antibody targeting one or more molecules expressed on lung epithelial cells.

In yet more particular embodiments, the nanoparticle comprises B7-S90, Sc12-E63, 50%/50% ratio of S90/Sc16; or B5-S3, Sc12-E39, 70%/30% ratio of S3/Sc16.

In certain embodiments, the compound of formula (I) comprises greater than 50% of the dry particle mass.

In some embodiments, the nanoparticle further comprises one or more additional compounds selected from formula (I) and/or lipid-polyethylene glycol (PEG). In certain embodiments, the lipid-PEG is selected from the group consisting of 1,2-dimyristoyl-rac-glycero-3-methoxypolyethylene glycol 2000 (DMG-PEG2k) and C18-PEG2k. In particular embodiments, the lipid-PEG comprises DMG-PEG2k.

In certain embodiments, the nanoparticle comprises a mass percent of lipid PEG from about 2 wt % to about 10 wt %, including about 2, 3, 4, 5, 6, 7, 8, 9, and 10 wt %.

In certain embodiments, a zeta-potential of the nanoparticle varies with a weight percent of lipid-PEG, wherein the zeta-potential has a range of −12 mV to +18 mV, including about −12, −11, −10, −9, −8, −7, −6, −5, −4, −3, −2, −1, 0, +1, +2, +3, +4, +5, +6, +7, +8, +9, +10, +11, +12, +13, +14, +15, +16, +17, and +18 mV, and in some embodiments between −5 mV to +5 mV, including about −5, −4, −3, −2, −1, 0, +1, +2, +3, +4, and +5 mV. In some embodiments, the zeta-potential is measured under an aqueous condition, for example, in 150 mM Phosphate Buffered Saline (PBS).

In certain embodiments, the nanoparticle comprises a plurality of nanoparticles having a polydispersity of less than about 0.2, including about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, and 0.20.

In certain embodiments, the nanoparticle comprises at least three components selected from: (i) one or more of compounds of formula (I), (ii) one or more lipid-PEG, and (iii) one or more nucleic acids.

In certain embodiments, the nanoparticle comprises about a 30:1 ratio of a compound of formula (I) to the one or more nucleic acids.

In some embodiments, the one or more nucleic acids is selected from the group consisting of an oligonucleotide, a cyclic dinucleotide, plasmid DNA, linear DNA, siRNA, miRNA, and mRNA. In particular embodiments, the one or more nucleic acids comprise an mRNA.

In some embodiments, the nanoparticle further comprises one or more excipients. In certain embodiments, the one or more excipients include one or more cryoprotectants, one or more sugars or sugar alcohols, MgCl₂, and combinations thereof. In particular embodiments, the one or more cryoprotectants comprise a sugar. In more particular embodiments, the sugar is selected from the group consisting of glucose, fructose, sorbitol, mannitol, sucrose, trehalose, and raffinose. In particular embodiments, the one or more sugar alcohols comprise sorbitol. In certain embodiments, the nanoparticle is lyophilized. In certain embodiments, the nanoparticle comprises a storable powder. In some embodiments, the nanoparticle has at least one dimension in the range of about 50 nm to about 500 nm, or from about 50 to about 200 nm. Exemplary nanoparticles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo. In certain embodiments, the nanoparticle has a size between about 100 nm to about 150 nm in diameter, including 100, 105, 110, 115, 120, 125, 130, 135, 140, 145, and 150 nm in diameter.

In certain embodiments, the presently disclosed subject matter provides a pharmaceutical formulation comprising one or more nucleic acids and a poly(beta-amino ester) (PBAE) of formula (I) in a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

### B. Kits

In some embodiments, the presently disclosed subject matter provides a kit comprising one or more of: one or more compounds of formula (I), one or more nucleic acids, one or more lipid PEGs, one or more reagents, and instructions for use.

In certain embodiments, the disclosed kits comprise one or more containers, including, but not limited to a vial, tube, ampule, bottle and the like, for containing the pharmaceutical composition including one or more compounds of formula (1). The compounds of formula (I) may be solvated, in suspension, or powder form, and may then be reconstituted in the pharmaceutically acceptable carrier to provide the pharmaceutical composition. The one or more containers also can be carried within a suitable carrier, such as a box, carton, tube or the like. Such containers can be made of plastic, glass, laminated paper, metal foil, or other materials suitable for holding medicaments.

In certain embodiments, the container can hold a pharmaceutical composition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### C. Methods for Delivering One or More Nucleic Acids to a Subject

In some embodiments, the presently disclosed subject matter provides a method for delivering one or more nucleic acids to a subject, the method comprising administering a presently disclosed nanoparticle or vaccine to the subject. In certain embodiments, the one or more nucleic acids is selected from the group consisting of an oligonucleotide, a cyclic dinucleotide, plasmid DNA, linear DNA, siRNA, miRNA, and mRNA. In particular embodiments, the one or more nucleic acids comprise an mRNA. In certain embodiments, the administering is selected from the group consisting of intramuscular, systemic, intranasal, intraocular, and intracranial.

### D. Method of Gene Editing

In other embodiments, the presently disclosed subject matter provides a method for gene editing, comprising contacting a cell with the nanoparticle as disclosed hereinabove.

In certain embodiments, the gene-editing protein directs site-specific target DNA disruption, mutation, deletion, or repair.

In certain embodiments, the nanoparticle and cell are contacted in vivo. In other embodiments, the nanoparticle and cell are contacted ex vivo.

In certain embodiments, the cell is a mammalian cell. In particular embodiments, the mammalian cell is a human cell.

### E. Methods of Treatment

In some embodiments the presently disclosed subject matter provides a method for treating or presenting a disease or condition, the method comprising administering a presently disclosed nanoparticle or vaccine to a subject in need of treatment thereof. In certain embodiments, the one or more nucleic acids is selected from the group consisting of an oligonucleotide, a cyclic dinucleotide, plasmid DNA, linear DNA, siRNA, miRNA, and mRNA. In particular embodiments, the one or more nucleic acids comprise an mRNA. In certain embodiments, the administering is selected from the group consisting of intravenous, intranasal, intramuscular, intraocular, and intracranial. In certain embodiments, the subject is a human or an animal.

More particularly, in some embodiments, the presently disclosed subject matter provides a method of treating a genetic disease, condition, or disorder, the method comprising administering a therapeutically effective amount of a nanoparticle disclosed hereinabove to a subject in need of treatment thereof.

In certain embodiments, the disease, condition, or disorder comprises cystic fibrosis (CF). In particular embodiments, the subject being treated for cystic fibrosis has a canonical splice site variant. In certain embodiments, the canonical splice site variant introduces a premature stop codon (PTC). In certain embodiments, the canonical splice variant comprises an alterations of the 5′GT splice motif or 3′AG splice motif. In particular embodiments, the canonical splice site variant comprises c.2988+1G>A. In more particular embodiments, the treatment corrects c.2988+1G>A in airway epithelia of the subject. In certain embodiments, the subject is of African descent.

In certain embodiments, the method further comprises administering a protein-targeted modulator therapy.

In certain embodiments, the method comprises a systemic administration of the nanoparticle. In particular embodiments, the systemic administration is intravenous (IV) or intranasal (IN).

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed composition of formula (I), to block, partially block, interfere, decrease, or reduce the severity of the disease state. Thus, one of ordinary skill in the art would appreciate that the term “inhibit” encompasses a complete and/or partial decrease in the severity of a disease state, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I) described herein and at least one other therapeutic agent, such as a protein-targeted modulator therapy. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compositions described herein can be administered alone or in combination with adjuvants that enhance stability of the compositions alone or in combination with one or more therapeutic agents, facilitate administration of pharmaceutical compositions containing them in certain embodiments, provide increased dissolution or n dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound of Formula (I) described herein and at least one additional therapeutic agent can be varied so long as the beneficial effects of the combination of these agents are achieved. Accordingly, the phrase “in combination with” refers to the administration of a compound of Formula (I) described herein and at least one additional therapeutic agent either simultaneously, sequentially, or a combination thereof. Therefore, a subject administered a combination of a compound of Formula (I) described herein and at least one additional therapeutic agent can receive a and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the described herein and at least one additional therapeutic agent are administered simultaneously, they can be administered to the subject as separate pharmaceutical compositions, each comprising either a compound of Formula (1) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an miR-described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

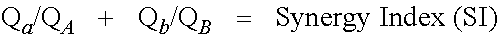

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Base Editing as a Treatment Approach for Correction of C.2988+1G>a, the Most Common CF-Causing Variant in Individuals of African Descent

Alterations of the 5′GT splice motif or 3′AG splice motif result in severe missplicing and are prevalent in many genetic diseases, including cystic fibrosis (CF). While protein-targeted modulator therapies are currently available for treatment of CF, individuals with these canonical splice site variants (CSSVs) are among the approximately 10% who remain untreated. The most common CF-causing variant in individuals of African descent is a CSSV, c.2988+1G>A. While >75% of individuals with a CSSV have a modulator eligible in trans allele, only 50% of the 450 individuals bearing c.2988+1G>A are eligible. Thus, there is a particular unmet need for a treatment for these individuals.

CRISPR/Cas9-mediated adenine base editing (ABE) is an efficient and targeted genome editing method to correct G>A variants. We electroporated NRCH-ABE8e mRNA and a previously optimized sgRNA to non-differentiated human primary nasal (HNE) or bronchial (HBE) epithelial cells from individuals with CF compound heterozygous for c.2988+1G>A. After differentiation of edited cells genomic editing and recovery of CFTR channel function were assessed. In primary HBEs and primary HNEs, we observed an allelic conversion to WT of 74.7% and 81.3%, respectively, at the +1 site. Interestingly, we also observed high levels of editing at adjacent adenines (+3, +7), which would have a modest effect on mRNA splicing (approximately 20% reduction). This did not preclude recovery of CFTR channel function, however. Compared to WT/WT HBEs and HNEs, unedited cells showed approximately 5% function, while edited cells achieved greater than 50% function.

Since electroporation is not a translationally viable delivery approach, we investigated polymeric nanoparticle mediated delivery to both primary HBE and HNE cells by flow cytometry. GFP mRNA evaluated transfection efficiency and cell viability across three dosages (150, 75, 32.5 ng), four polymer-to-mRNA weight-to-weight ratios (60, 40, 30, 20) and three polymers (R, X, Y). HBEs transfected with polymers R and Y showed approximately 57% transfection at 75 and 32.5 ng, with polymer X averaging approximately 25% across the same dosages. Polymer Y showed slightly higher viability of HBEs vs. polymer R. HNEs in comparison achieved a maximum of approximately 37% GFP transfection with polymers R and Y at 32.5 ng compared to a maximum of approximately 22% with polymer X at the same dose, with 75-95% viability across all polymers.

The ABE design reported here corrects c.2988+1G>A in airway epithelia with high efficiency when robust delivery is achieved. Given that nanoparticle optimization allowed successful delivery to greater than 50% of cells, we anticipate clinically significant recovery of function in vivo by combining this ABE design with an optimized polymeric nanoparticle.

### Example 2

### Polymeric Nanoparticle-Mediated Base Editing in Immortalized Cystic Fibrosis Cells with a Rare Variant

### 2.1 Materials and Methods

Poly(beta-amino ester)s were synthesized by Michael addition, fabricated into nanoparticles by electrostatic self-assembly with mRNA/DNA, and evaluated for gene delivery by flow cytometry. mRNA-encoded GFP served as a reporter to evaluate transfection efficiency, cell viability and mean fluorescence intensity (MFI) across three dosages (150, 75, 32.5 ng mRNA), four polymer-to-mRNA weight to weight (w/w) ratios (60, 40, 30, 20) and four different polymers. CF bronchial epithelial (CFBE) cells stably expressing the 3120+1G>A expression minigene were transfected with the optimized nanoparticle formulation to deliver adenine base editor (ABE) and guide RNA (gRNA) at two dosages (150, 75 ng) of mRNA and DNA. Reconstitution of CFTR function can be measured by short circuit current (I_{sc}). After stabilization of transepithelial current, forskolin is added to increase cAMP levels which in turn stimulates activation of CFTR, followed by administration of CFTR inhibitor-172 to block CFTR-mediated currents. The corresponding drop in current is a CFTR-specific measurement of function. See Moran, 2008.

### 2.2 Results and Discussion

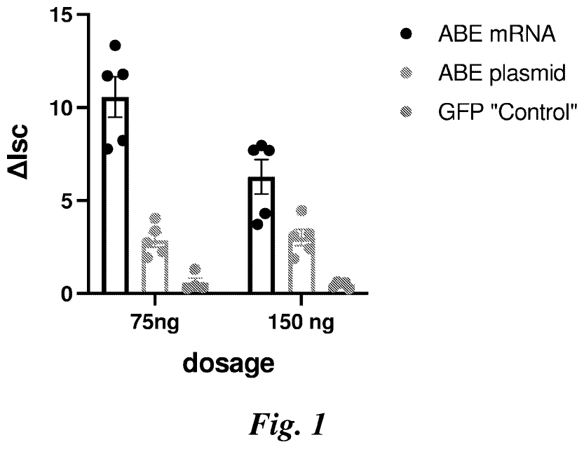

Flow cytometry showed polymer R achieved >85% GFP transfection compared to a maximum of approximately 35% across the other three polymers at the maximum 150 ng dosage, with approximately 80% viability normalized to untreated cells. In addition, Polymer R achieved GFP MFI over one order of magnitude higher compared to other formulations (approximately 30,000 vs 2,700 MFI respectively) across the other three polymers at 150 ng dosage and 40 w/w ratio. CFBE cells transfected with polymer R nanoparticles containing ABE mRNA and gRNA at 75 ng and 150 ng showed CFTR function increase to 10.6±1.1 μA (mean±SEM; approximately 10% of wild type) and 6.3±0.9 μA (approximately 6% of wild type), respectively (FIG. 1). Increased toxicity at the higher dosage could explain the larger increase in CFTR current at the lower dosage. DNA encoded ABE plasmid and gRNA showed a less robust increase in CFTR function (2.9±0.4 μA and 3.0±0.4 μA for 75 and 150 ng doses respectively) (FIG. 1). This was likely a result of the nanoparticle formulation being optimized for RNA instead of DNA cargo or the additional intracellular barriers that must be overcome for successful DNA delivery.

### 2.3 Summary

The presently disclosed subject matter demonstrates that an optimized polymeric nanoparticle formulation containing ABE and gRNA can result in recovery of CFTR function in isogenic cells bearing the 3120+1G>A CFTR variant. We are adapting these nanoparticles for RNA and DNA encoding ABE and gRNA delivery to primary HBE and HNE cells.

### Example 3

### Polymeric Nanoparticles for Nonviral Nucleic Acid Delivery

### 3.1 Nanoparticle Structure

Referring now to FIG. 2A and FIG. 2B is a representative scheme for preparing the presently disclosed nanoparticles and chemical structures of representative polymers. In some embodiments, the polymer used in animal studies is:

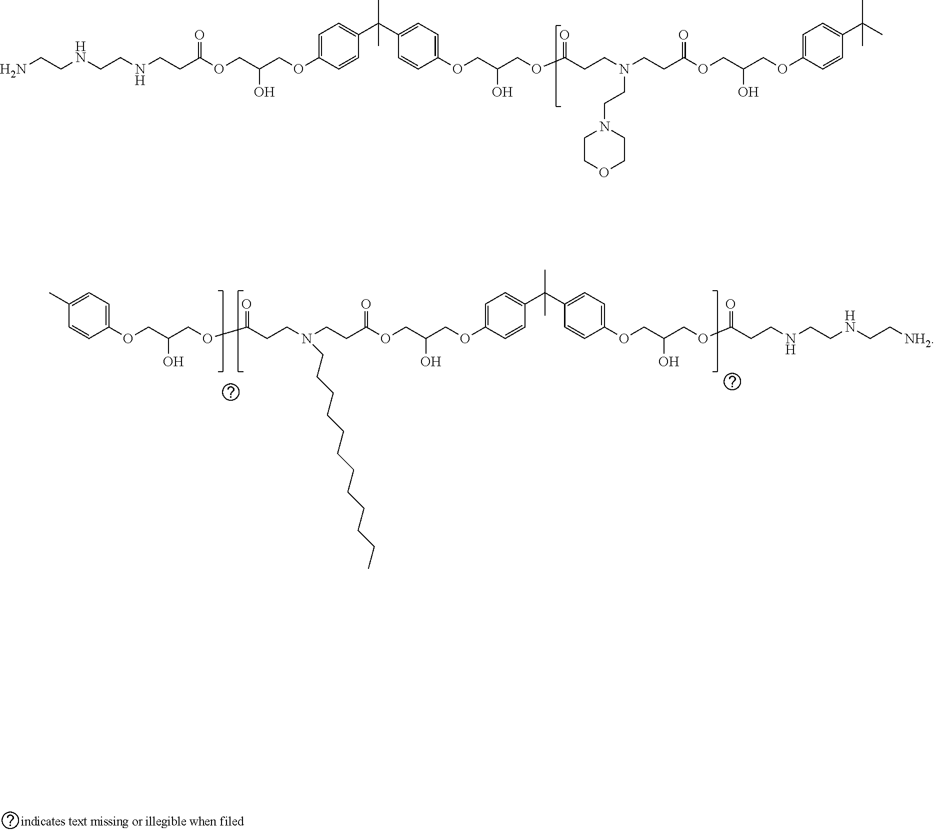

Variations on this structure, including those using end-capping monomers E1, E39, and E58, also were tested in vivo and found to be effective at transfecting the lungs while causing no observed toxicity.

### 3.2 Nanoparticle Formulation

To maximize gene delivery to lung epithelium rather than to endothelial cells or phagocytic immune cells, nanoparticles were designed to be approximately about 100 nm to about 150 nm in diameter and stabilized with poly(ethylene glycol) (PEG)-lipid. To further improve targeting of epithelial cells, nanoparticles were functionalized with an antibody specific for EpCAM (aka CD326), a molecule expressed on epithelial cells, particularly of the lungs. This functionalization was accomplished using N-hydroxysuccinimide (NHS)-amine chemistry by first reacting a PEG-lipid-NHS molecule with the amine-containing anti-CD326 antibody to form a covalent bond between the PEG-lipid and the antibody. This PEG-lipid antibody was then used to cap and functionalize the surface of small (e.g., 100 nm to 150 nm) nanoparticles.

### 3.3 Animal Studies: Reporter Genes

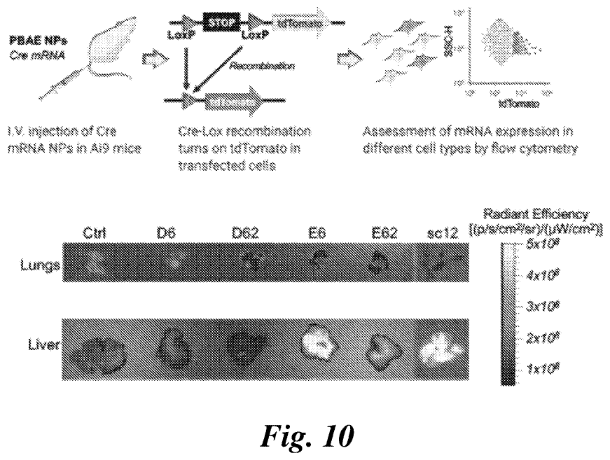

Referring now to FIG. 10, in Ai9 reporter mice, successful transfection of Cre mRNA results in stable expression of fluorescent tdTomato, allowing sensitive detection of specific cell types.

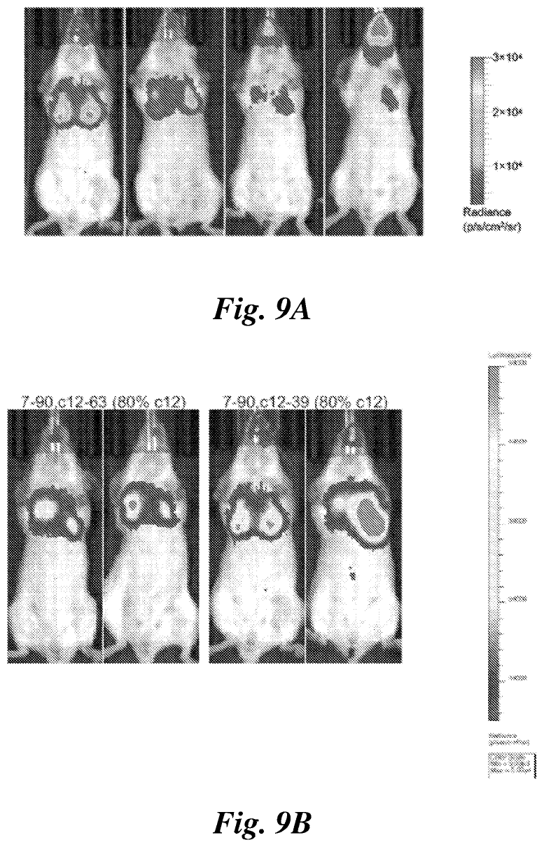

Referring now to FIG. 9B, intranasal (IN) administration of nanoparticles leads to strong expression of luciferase reporter gene in the lungs.

### 3.4 Flow Cytometry on Excised Lungs

In this example, antibody staining allows identification of specific cell populations. Nanoparticles were either: (a) administered intravenously (IV) or intranasally (IN); non-functionalized or surface-functionalized with an anti-CD326 antibody; and/or administered once (1×) or three times (3×).

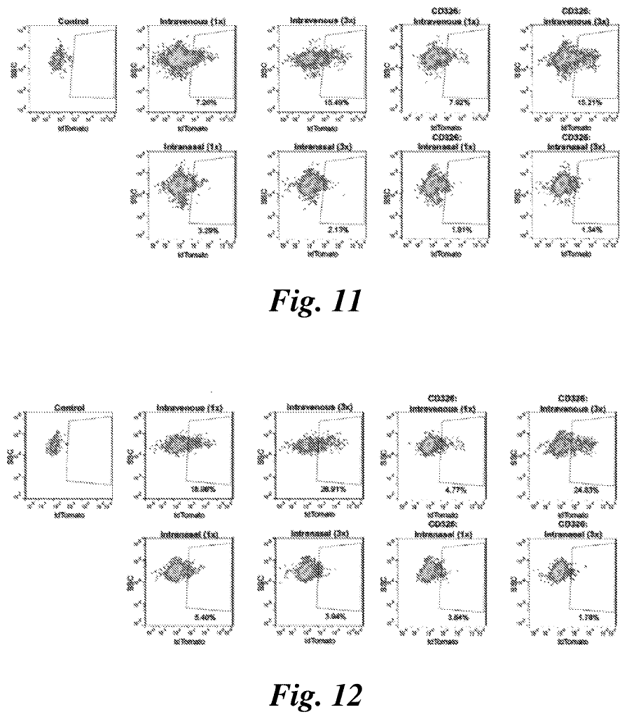

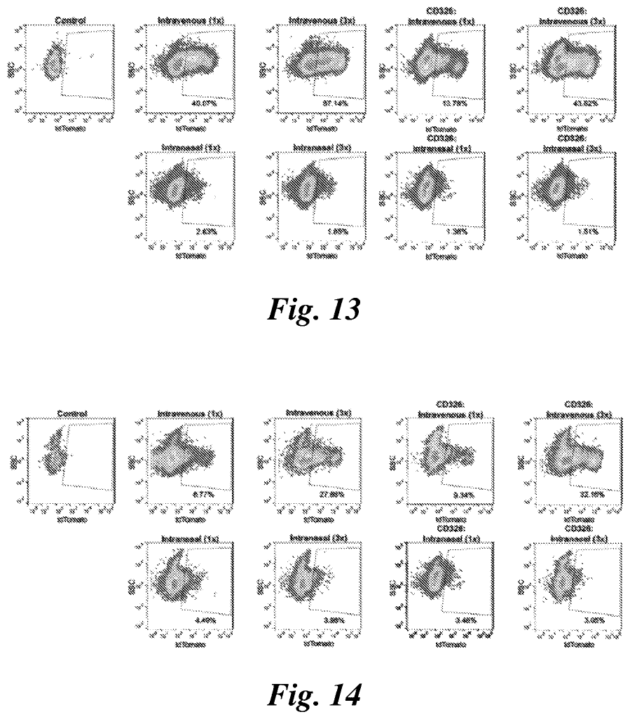

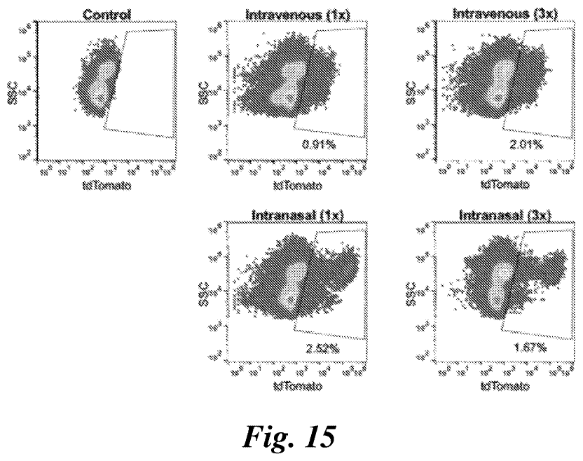

Referring now to FIG. 11, FIG. 12, FIG. 13, FIG. 14, and FIG. 15, IV delivery was superior to IN delivery for transfection epithelial, endothelial, and stromal cells of the lungs. Multiple injections were well tolerated and improved transfection efficacy in most cell populations. Addition of the CD326 antibody improved the specificity of transfection of epithelial and stromal cell populations compared to other commonly transfected populations like the endothelium.

### Example 4

### Poly(β-Amino Ester) (PBAE)s for Systemic Gene Delivery

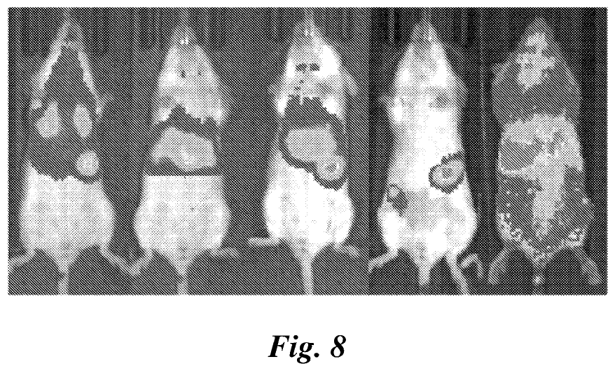

Referring now to FIG. 8, different PBAE structures can efficiently transfect various organs after systemic injection. In this example, a primary target is the lung epithelium. It has been shown previously that small changes to polymer end-cap structure can affect transfection efficacy and organ distribution and polymer hydrophobicity can be tuned to target transfection in certain organs. Rui et al., 2022.

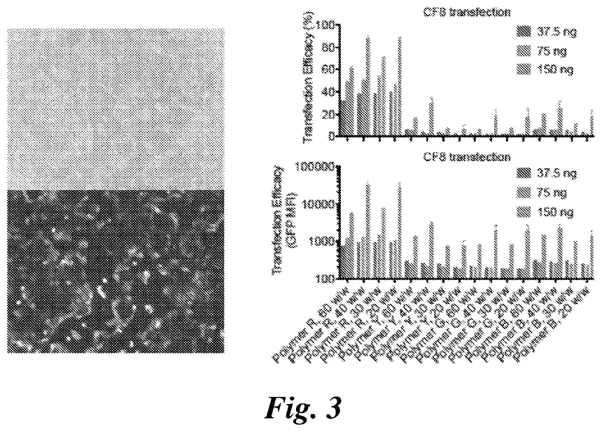

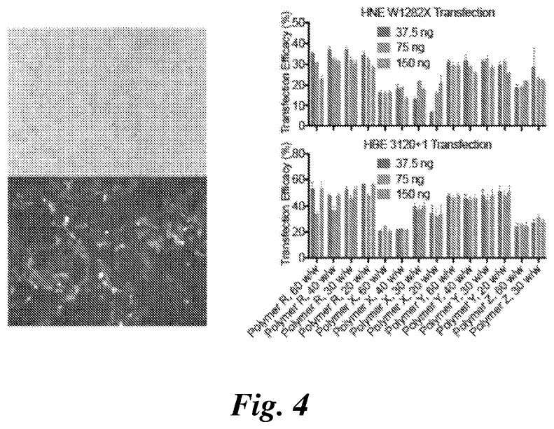

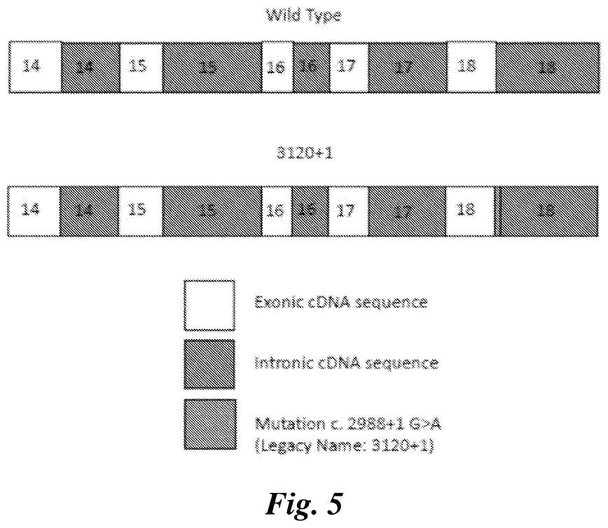

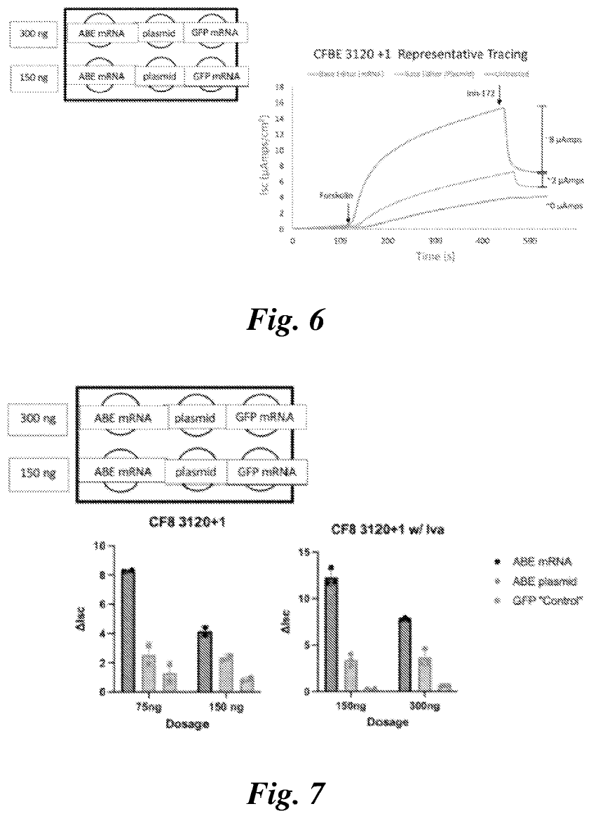

Referring now to FIG. 3-FIG. 7, FIG. 3 shows in vitro transfection of CF8 3120+1 stable cells. FIG. 4 shows in vitro transfection of primary nasal or bronchial epithelial cells. FIG. 5 shows in vitro editing of CF8 3120+1 stable cells. FIG. 6 shows in vitro editing of CF8 3120+1 stable cells. Short-circuit tracings show restoration of CFTR function in transfected cells, indicating successful editing. FIG. 7 shows in vitro editing of CF8 3120+1 stable cells. In combination with ivacaftor, edited cells achieved approximately 12 μAmp current gain, with about 8% to about 10% WT restoration;

FIG. 9A demonstrates that IN administration provides direct access to lungs, is non-invasive and easy to administer, and is unlikely to reach tissues outside the lungs. FIG. 9B demonstrates that IN administration of nanoparticles leads to strong expression of luciferase reporter gene in the lungs. FIG. 10 demonstrates in vivo transfection of specific cells.

Referring now to FIG. 11-FIG. 15, the transfection of different lung cells. FIG. 11 show transfection of lung epithelial cells: CD45−CD31−CD326+. FIG. 12 shows transfection of lung bronchial epithelial cells: CD45−CD31−CD326+CD24+. FIG. 13 shows transfection of lung endothelial cells: CD45−CD31+. FIG. 14 shows transfection of lung stromal cells: CD45−CD31−CD326−. FIG. 15 shows transfection of lung leukocytes: CD45+;

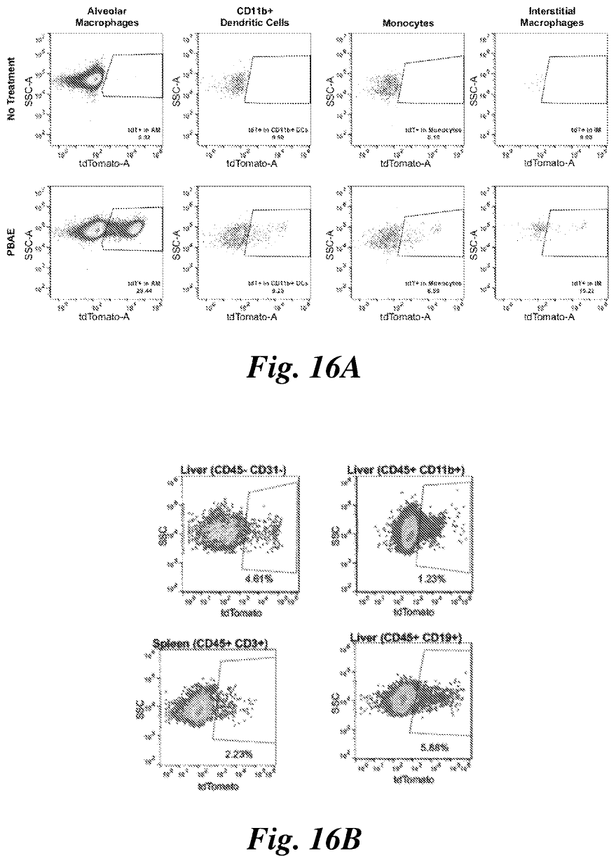

FIG. 16 demonstrates that systemic administration of NPs successfully edits not only epithelial cells of the lungs, but also other cell types in other organs, including liver (CD45−, CD31−), liver (CD45+, CD11b+), spleen (CD45+, CD3+), and liver (CD45+, CD19+); and

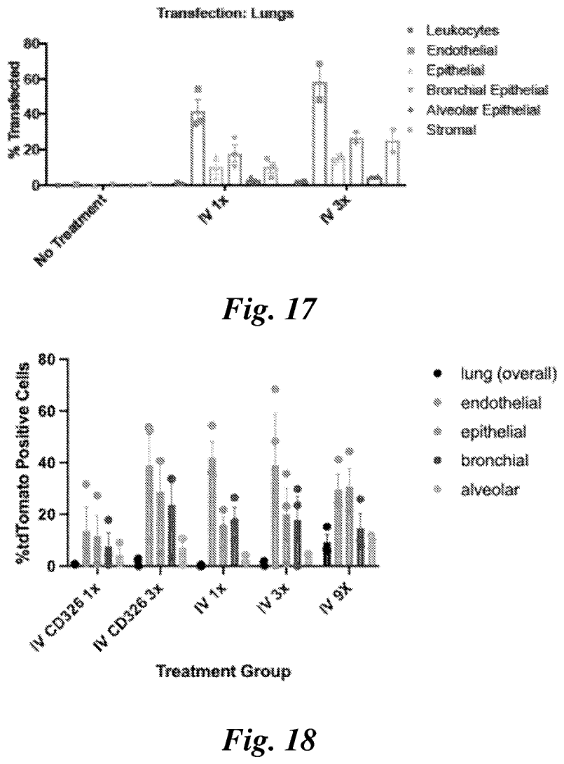

FIG. 17 demonstrates in vivo transfection of lung cells. PBAE nanoparticles transfect endothelial cells and bronchial epithelial cells in the lungs after IV administration. Multiple administrations were well tolerated and improved transfection efficiency.

In summary, the presently disclosed subject matter demonstrates that PBAEs can be used for gene editing. PBAEs can form nanoparticles with nucleic acids. A combinatory library can be screened in vitro to find polymers with high transfection efficacy. Cas9 or other editors and sgRNA can be encoded in DNA or mRNA. Importantly, PBAE nanoparticles can be used for gene editing in vivo. In one embodiments, ABE delivered to CF epithelial cells leads to gain of function. PBAEs transfect lung epithelium and a wide range of other cells after systemic injection. PBAE NPs can be administered multiple times for increased efficacy. Future steps include track proportion of transfected cells over time and in vivo editing in CF mouse model.

### Example 5

### Nanoparticles Delivered to Primary Human Airway Epithelial Cells Allows for Targeted Systemic Lung Delivery

### 5.1 Background

Current treatments for cystic Fibrosis (CF) primarily focus on modulator drug therapies designed to correct malfunctioning CFTR protein. Cutting, 2015. These modulators, however, are ineffective for the 10% of CF individuals with variants that do not allow protein production. Gene editing of these variants would allow production of CFTR protein and restoration of chloride channel function.

### 5.2 Overview

In this Example, representative poly(beta-amino ester) (PBAE) nanoparticles have been screened against primary airway epithelial cells cultured from both CF and healthy individuals to aid in a formulation that will target these cells following systemic delivery. Systemic delivery using an optimized NP formulation to the Ai9 mouse model transfects both endothelial and epithelial cell types. In vitro, electroporation of a modified CRISPR-Cas9 base editor mRNA to primary human nasal epithelial (HNE) cells carrying 3120+1G>A/G480S achieves 81% correction of the 3120+1 G>A allele. As this nonviral transfection results in CFTR function within or above the range of WT values observed in WT HNEs, Joynt et al., 2023, in-depth single cell RNA (scRNA) sequencing was conducted across edited and unedited samples. This gene editing approach is promising for new, durable CF treatment.

### 5.3 Materials and Methods

PBAEs were synthesized via Michael addition, fabricated into nanoparticles by electrostatic self-assembly with mRNA, and evaluated for gene delivery by flow cytometry. mRNA-encoded GFP served as a reporter to evaluate transfection efficiency, cell viability and mean fluorescence intensity (MFI) across three dosages (150, 75, 32.5 ng mRNA), four polymer-to-mRNA weight to weight (w/w) ratios (60, 40, 30, 20) and four different polymers. Primary human airway epithelial cells from CF and healthy donors were seeded in 96-well plates then transfected with nanoparticles before undergoing microscopy and flow 24 hours post transfection. Nanoparticles carrying Cre mRNA were delivered via tail vein injection to Ai9 mice at three different intervals (1×, 3×, 9×). Lungs were dissociated and antibody stained before undergoing flow. All flow analysis was completed in FlowJo. Primary HNE cells (described above) were co-electroporated with ABE8e/sgRNA, then allowed to differentiate for 21 days on ALI culture before being dissociated for single cell library preparation. This was performed using 10× Genomics v3.1 3′ Library Preparation chemistry with dual indexing system. Sequencing was performed by the Johns Hopkins Genomic Resources Core Facility via paired-end sequencing 2×100 cycles using a NovaSeq 6000 Illumina sequencing machine. Analysis of scRNA-seq results was performed using 10× Genomics Cell Ranger 3.1.0 followed by the Seurat package (version 4.1) in R created by the Satija Lab. Hao et al., 2021. Subsetting was determined based on comparisons of the number of genes expressed, UMI count, and percentage of reads mapped to mitochondrial RNA. A log normalization with a scale factor of 10,000 was used.

### 5.4 Results, Conclusions and Discussions

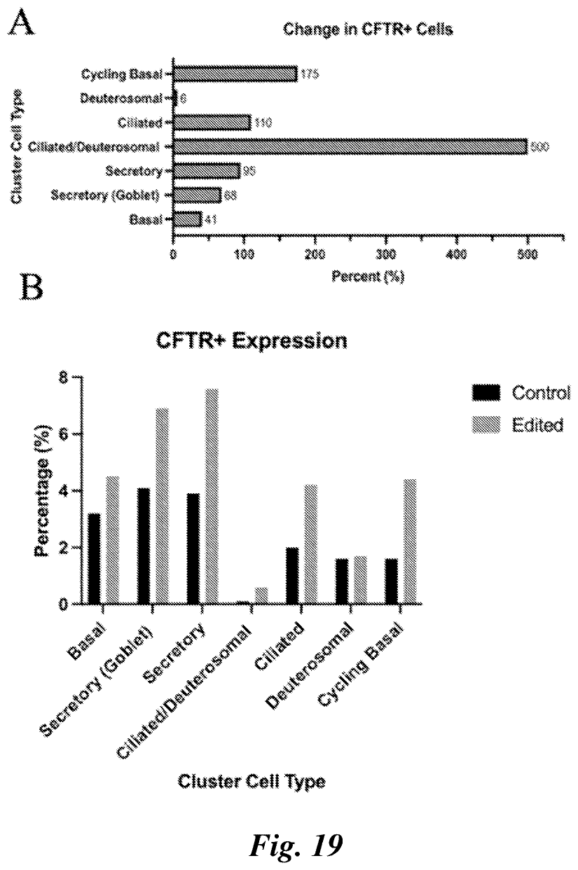

Primary human bronchial epithelial cells from a CF and healthy individual achieved 40-60% GFP transfection, with primary human nasal epithelial cells from a CF individual achieving approximately 35% transfection and that from a healthy donor achieving approximately 85% transfection. Viability for the two lowest dosages was consistently between 70-85%. Systemic delivery to the Ai9 mouse model showed consistent increase in tdTomato+ cells across all lung cells as the number of injections increased, from 0.8 to 1.9 to 9% at 1×, 3× and 9× dosing respectively. Additionally, the number of epithelial cells transfected consistently increased across dosing, with the number of endothelial and epithelial tdTomato+ cells ranging from 15-40% and 15-35% respectively (FIG. 18). Analysis of scRNA sequencing of 8,791 cells across two samples of edited and unedited primary HNEs compared CFTR-positive cells as well as expression levels. There was a consistent increase in CFTR+ cells between unedited and edited cells across all defined cell type clusters, most noticeably in ciliated/deuterosomal (500% increase) and cycling basal cells (175%) (FIG. 19A). Cells expressing CFTR increase from 2.5% of total cells in unedited to 3.96% in edited and normalized read count per sample increased from 0.049 to 0.081. Editing increased the % of basal and cycling basal cells expressing CFTR, indicating correction was achieved in airway progenitor cells (FIG. 19B).

One embodiment of the presently disclosed PBAE nanoparticle formulation achieved up to 85% transfection rate in healthy primary human nasal epithelial cells while maintaining around 75% viability. This same polymer achieving 20-40% transfection of endothelial and epithelial tissues with systemic delivery in an Ai9 model. Base editing of primary HNE cells carrying the 3120+1G>A variant show levels of CFTR function after editing equivalent to >50% WT, Cutting et al., 2015, exceeding the established clinically significant threshold of ˜10%. Here we demonstrate that editing is occurring in progenitor cells after electroporation-mediated delivery of RNA-encoded base editing reagents to undifferentiated primary HNEs. In ongoing work, we are preparing more samples for scRNA analysis of edited and unedited primary cells, as well as further investigating targeted cell types during in vivo delivery to the Ai9 mouse model using FACS and scRNASeq.

### Example 6

### Optimized Nanoparticle-Mediated Base Editing of CF-Causing Variants

### 6.1 Background

Modulator therapies are ineffective for the 10% of CF individuals with variants that do not allow for protein production. Among these is the canonical splice variant 3120+1 G>A, the second most common CF-causing mutation in African Americans. Macek et al., 1997. Gene editing to correct 3120+1 would allow for production of CFTR protein and enhancement of function using available CFTR modulators. Sharma et al., 2018. In CRISPR-based approaches, a double strand break can lead to indel formation and off target genome instability. Eid et al., 2018.

### 6.2 Overview

This Example provides an alternative method using modified CRISPR-Cas9 adenine base editors (ABEs) which initiate repair by a single-strand nick, allowing for lower off-target effects and more efficient editing. Komor et al., 2016. Nanoparticle-mediated delivery of mRNA has been shown to be highly successful, with safety and efficacy for systemic delivery demonstrated by the next-generation SARS COV-2 vaccines. Polack et al., 2020; Baden et al., 2021.

More particularly, this Example evaluates the effectiveness of several new poly(β-amino ester) (PBAE) nanoparticle formulations (FIG. 2A) at delivering green fluorescent protein (GFP) mRNA to primary and immortalized CF bronchial epithelial (CFBE410-) cells. Using an optimized nanoparticle formulation, we then tested correction efficacy of the 3120+1G>A (c.2988+1 G>A) variant using short circuit to measure CFTR functional gain. Finally, this optimized nanoparticle formulation was tested in vivo with an Ai9 mouse model (FIG. 10).

### 6.3 Methods and Materials

GFP served as a reporter to evaluate transfection efficiency, cell viability and mean fluorescence intensity (MFI) across three dosages (150, 75, 32.5 ng mRNA), four polymer-to-mRNA weight to weight (w/w) ratios (60, 40, 30, 20) and four different polymers (R, Y, G, B) formulated as shown in FIG. 2A. 7-AAD served as a live/dead stain to quantify viability, with flow cytometry results being analyzed using FlowJo software.

CFBE cells stably expressing the 3120+1G>A expression minigene (EMG) were transfected with the optimized nanoparticle formulation to deliver ABE and guide RNA at two dosages (150, 75 ng) of mRNA and DNA. Reconstitution of CFTR function can be measured by short circuit current (Isc). After stabilization of transepithelial current, forskolin is added to increase cAMP levels, which in turn, stimulates activation of CFTR, followed by administration of CFTR inhibitor-172 to block CFTR-mediated currents. The corresponding drop in current is a CFTR-specific measurement of function. Moran and Zegarra-Moran, 2008. FIG. 6 demonstrates the gain of function seen in chloride ion flow across CFBE cells transfected with Polymer R nanoparticles, ABE and guide RNA when compared to untreated cells.

Ai9 mouse models were injected with PEG-coated PBAE nanoparticles encapsulating Cre mRNA to determine the factors that direct cell targeting. Intranasal (IN) and intravenous (IV) routes were tested with and without a targeting ligand (CD326) and single vs multiple doses (1× vs 3×). Single cell-level transfection could be detected by tdTomato expression, which was quantified using flow cytometry. In the lungs, tdTomato expression was evaluated in different cell types with antibody staining targeting epithelial cells.

### 6.4 Results

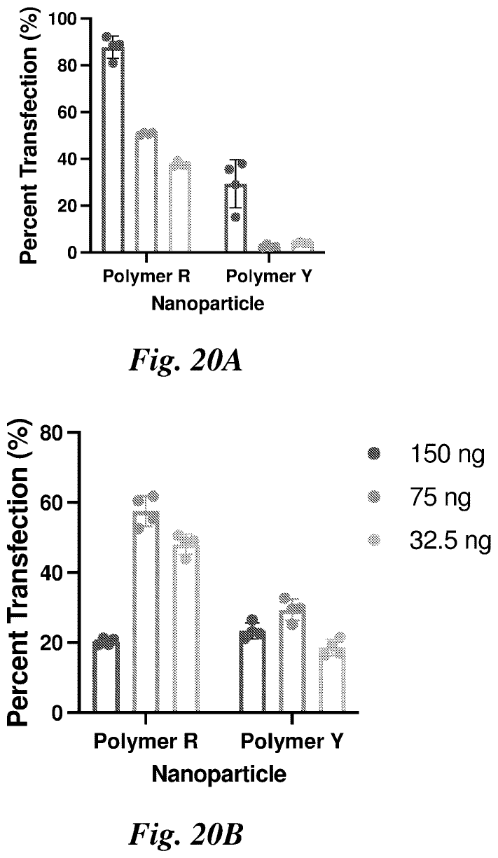

Flow cytometry showed polymer R achieved greater than 85% GFP transfection in CFBE cells compared to a maximum of approximately 35% across the other three polymers at the maximum 150 ng dosage (FIG. 20A), with approximately 80% viability normalized to untreated cells. In addition, Polymer R achieved GFP MFI over one order of magnitude higher compared to other formulations (approximately 150 vs 10 MFI respectively) when normalized to untreated cells across the other three polymers at 150 ng.

In primary human bronchial epithelial (HBE) cells, polymer R achieved >50% GFP transfection compared to a maximum of approximately 30% across the other three polymers at 75 ng (FIG. 20B), with approximately 85% viability normalized to untreated cells. Similarly, Polymer R achieved a GFP MFI double compared to other formulations (approximately 40 vs 20 MFI respectively) across the other three polymers at 75 ng.

CFBE cells transfected with polymer R nanoparticles containing ABE mRNA and guide RNA at 75 ng and 150 ng showed CFTR function increase to 10.6±1.1 μA (mean±SEM; ˜10% of WT) and 6.3±0.9 μA (approximately 6% of WT), respectively. DNA encoded ABE plasmid and guide RNA showed a less robust increase in CFTR function (2.9±0.4 μA and 3.0±0.4 μA for 75 ng and 150 ng doses respectively) (FIG. 1).

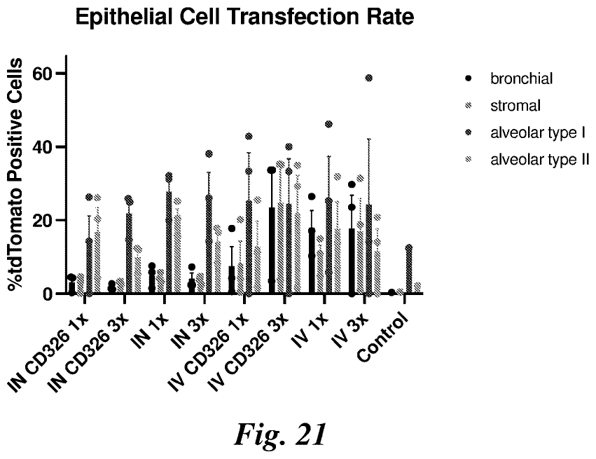

Distribution of tdTomato+ cells across different cell types in the lung are shown in FIG. 21. Using FlowJo we gated for markers specific to relevant airway epithelia cell types, most significantly bronchial, alveolar and stromal cell populations. Highest transfection was achieved at IV CDC326 3×, averaging approximately 25% and IV 1× and 3×, approximately 20%.

### 6.5 Discussion

The newly developed polymer R enables high transfection efficacy in both a CFBE cell line bearing an EMG with the 3120+1 variant as well as primary HBE cells. Our current formulations achieve greater than 50% transfection with 80% viability.

We have also been able to demonstrate an optimized nanoparticle formulation containing base editor and guide RNA can correct splicing of isogenic cells bearing the 3120+1 variant, resulting in recovery of CFTR function.

Finally, in vivo studies show systemic IV delivery of polymer R successfully targets relevant epithelia cell types. Next steps will include optimizing delivery and editing in primary HBE and HNE cells bearing the 3120+1 variant, as well as continuing systemic delivery optimization. If successful, our work could inform development of revolutionary therapies for minority CF patient populations who currently have no treatment options.

### 6.6 Summary

An optimized nanoparticle formulation containing ABE and guide RNA can correct splicing of isogenic cells bearing the 3120+1G>A CFTR variant, resulting in recovery of CFTR function to clinically relevant levels. Preliminary data from in vivo systemic delivery reveals targeting of relevant cell types that express CFTR. Nanoparticles will be tested for in vivo delivery of ABE mRNA and guide RNA in a mouse model bearing 3120+1 G>A.

### Example 7

### Polymeric Nanoparticle for Lung Targeting

This Example includes data related to: primary HNE/HBE nanoparticle screens; fLuc in C57BL/6 mice; and Ai9 mouse data, including additional dosages at 9× and a lung dissociation protocol, including 3×, 3× CD326, 6×, 6× CD326.

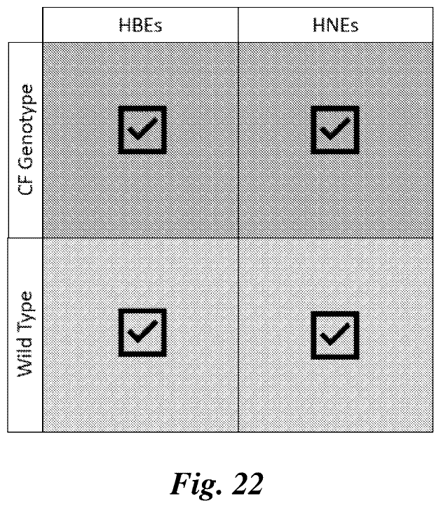

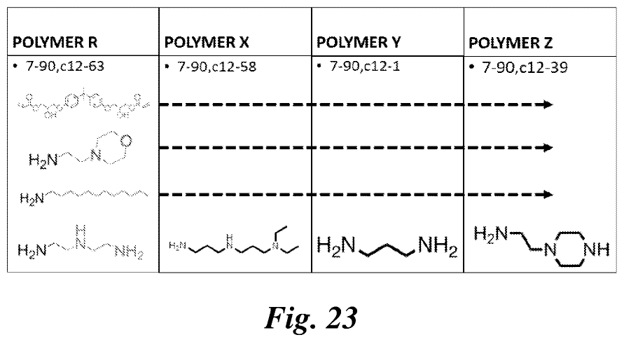

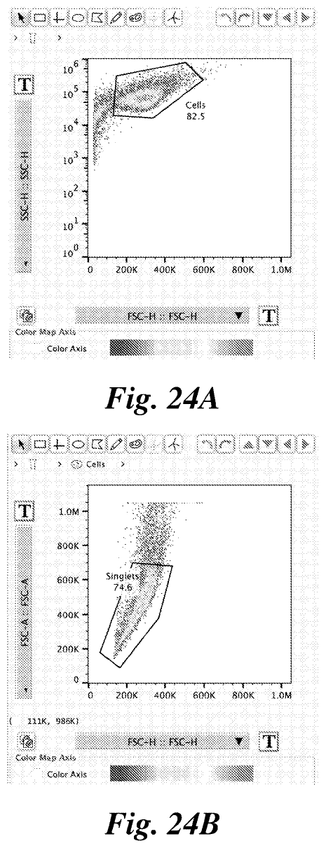

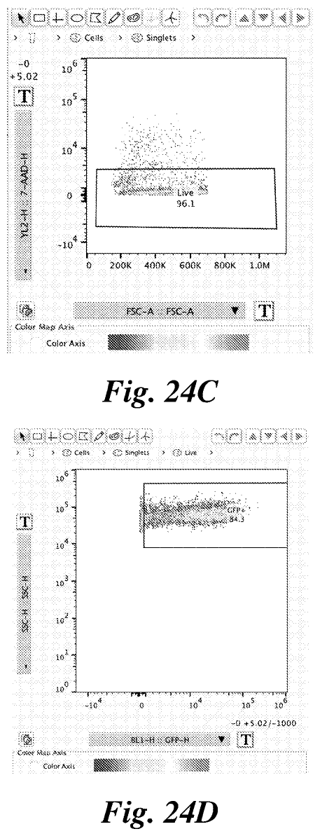

Referring now to FIG. 22, is a diagram showing a representative strategy for a primary human nasal epithelial (HNE)/human bronchial epithelial (HBE) nanoparticle screen with flow cytometry. Referring now two FIG. 23, are representative polymers used in this screen. In this screen three 96-well plates were coated with conditioned media. The HNE and HBE cells were transfected with four different nanoparticle compositions (Polymer R, X, Y, Z) at four different w/w ratios (60, 40, 30, 20) and three dosages (150 ng, 75 ng, 32.5 ng) of GFP mRNA. The flow cytometry gating breakdown is shown in FIGS. 24A-24D, in which cells (FIG. 24A), singlets (FIG. 24B), live (FIG. 24C), and GFP+ (FIG. 24D) are shown, respectively.

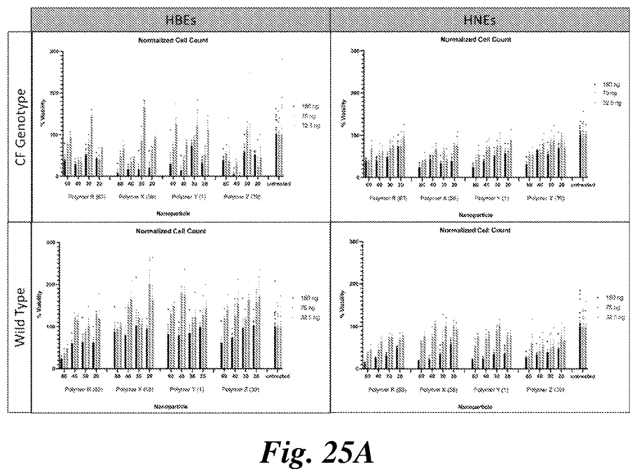

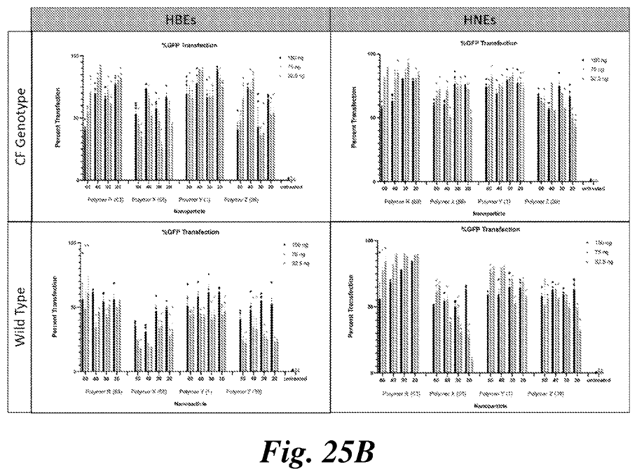

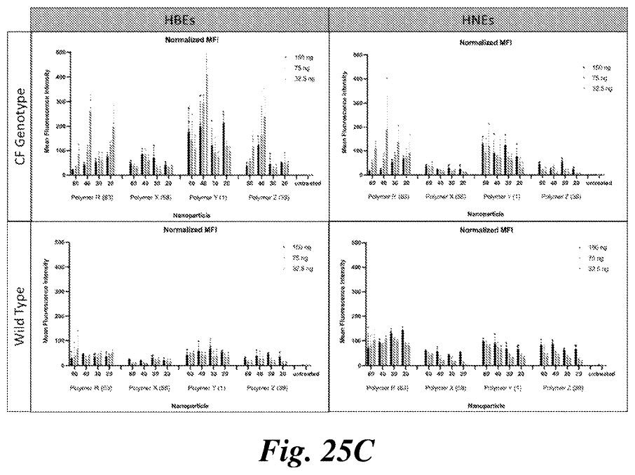

Viability was assessed using 7-Aminoactionmycin D (7-AAD) live/dead stain and normalized against untreated cells (treated*100/average (untreated)). Generally, viability was inversely proportional with dosage, with the largest viability drop seen in the 150 ng dosage (FIG. 25A). The 75 and 32.5 ng dosages generally had a similar effect on viability for both HBE samples. Any viability values over 100%, particularly for the HBE WT sample, were a result of variability between treated and untreated samples. Conditions with greater than 80% viability were considered not particularly toxic to cells.

HBEs were observed to have fewer live cells compared to previous flow analysis experiments when undergoing analysis using FlowJo. It was suspected this additional debris was due to the primary cells being particularly sensitive to toxicity and exposure to the nanoparticles. For this reason, both HNE samples underwent a media change two hours post transfection which increased live cell count and eliminated much of the observed debris. This also generally decreased SEMs compared to the HBE samples. HBEs achieved 40-60% GFP transfection, with primary HNEs from a CF individual achieving approximately 35% transfection and that from a healthy donor achieving approximately 85% transfection (FIG. 25B).

Mean fluorescence intensity (MFI) assessed copy number per cell. This was also normalized against untreated cells. MFI was directly proportional to dosage for both HNE samples excluding Polymer R, whereas for both HBE samples the MFI did not follow a general pattern but generally was highest at the intermediate 75 mg dosage (FIG. 25C). Most importantly, higher dosage did not necessary correlate with higher MFI, indicating that viability of cells is also an important factor to consider when determining optimal transfection conditions. Note, however, that MFI not a parameter of concern, with the more important aspect being delivering protein vs ABE8e.

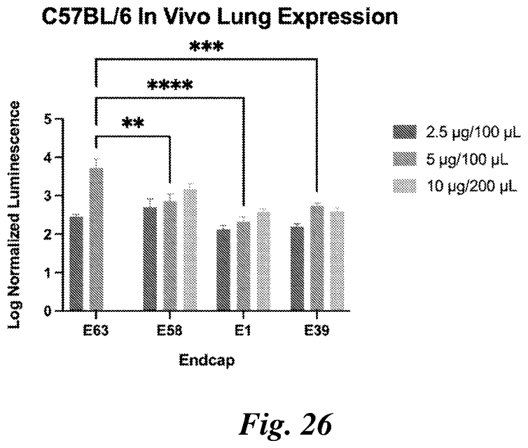

Referring now to FIG. 26, E63 (Polymer R) shows the highest luminescence readout during initial in vivo studies. In this example, NPs carrying fLuc mRNA were injected RO into C57BL/6 mice at the doses indicated, then lungs were collected and total bioluminescence was measured. The polymer selection notes is the same as those screened during in vitro primary HBE/HNE cell screens (see FIG. 23).

Three different lung tissue dissociation methods were evaluated in Balb/c mice. These protocols include:
P1=Protocol 1: Dispase paper (optimized for epithelial/progenitor populations); https://journals.physiology.org/doi/epdf/10.1152/ajplung.00334.2015;
P2=Protocol 2: GentleMACS lung kit (optimized for endothelial/epithelial populations) https://static.miltenyibiotec.com/asset/150655405641/document_p6m332m7v51bhcii3lk sd0on2c?content-disposition=inline; and
P3=Protocol 3: traditional Green lab protocol (optimized for immune populations). Flow cytometry was performed for a variety of cell populations.

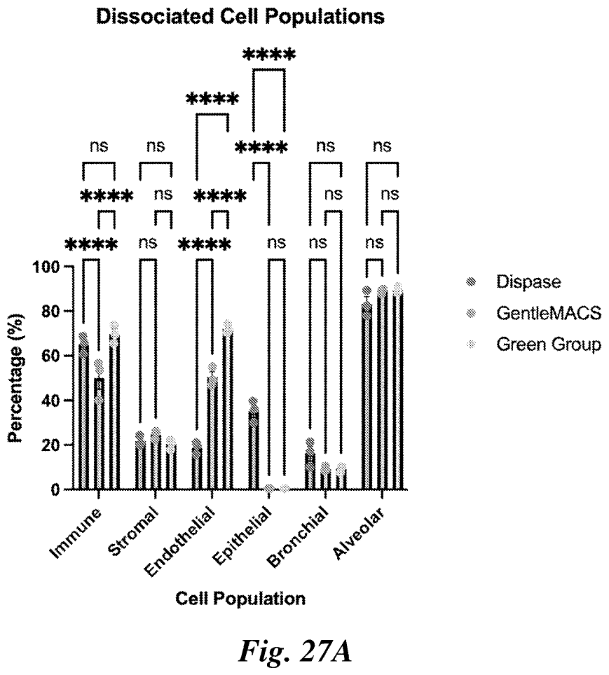

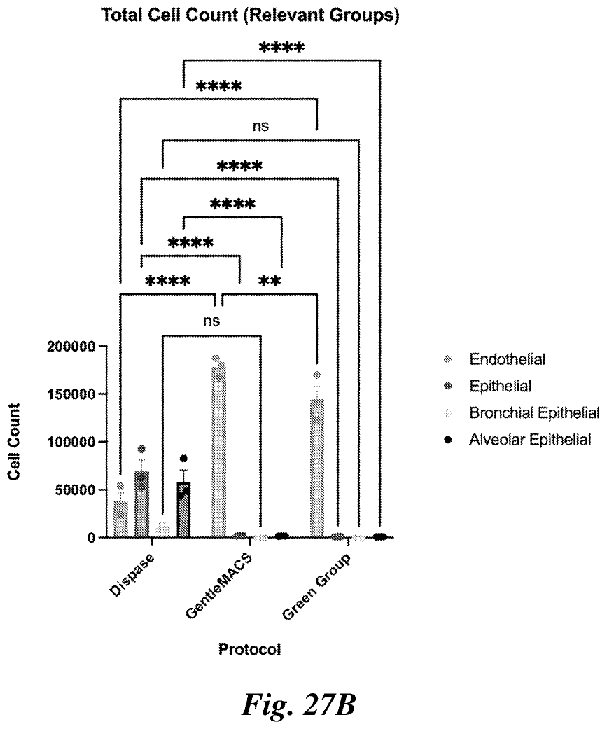

Referring now to FIG. 27A and FIG. 27B, the Dispase protocol is favored to obtain more epithelial cells in terms of both % and raw cell counts. Importantly, the Dispase protocol also does this without sacrificing immune cells. Note, however, that the Dispase protocol does sacrifice endothelial cells (CD31+CD326−). Conclusions based on ANOVA done on count groups, however, should be taken with caution. Numbers are wide ranging between cell population groups, which may throw off the data distribution. Based on these data, the preferred dissociation protocol by cell population categories appear to be: Immune: Green (%), GentleMACs (count); Stromal: GentleMACS (%, count); Endo: Green (%), GentleMACs (count); Epi: Dispase (%, count); BEC: Dispase (%, count); AEC: Gentle/Green (%), Dispase (count; *significantly); CD31+CD326+: Dispase (%, count); and CD31−CD326−: GentleMACS (%, count).

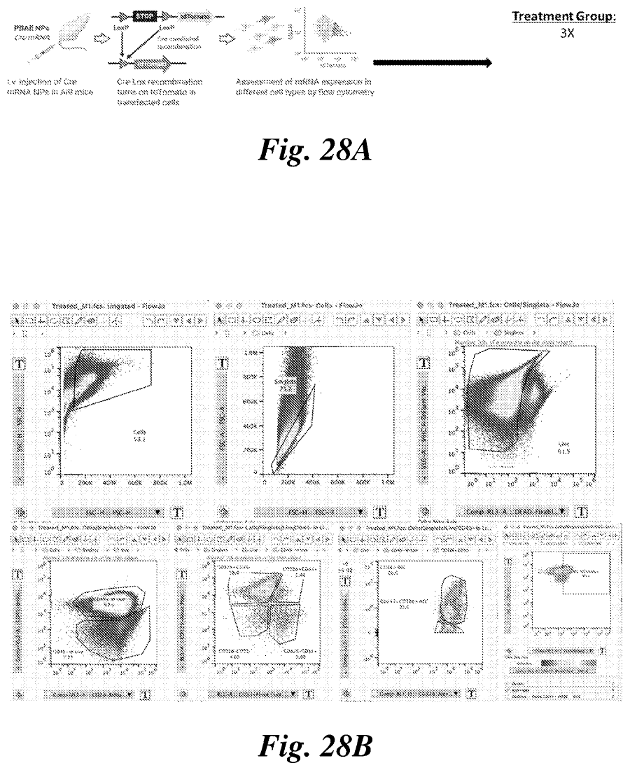

Referring now to FIG. 28A, in an Ai9 mouse study, N=6; 5 μg NPs injected 3×; N=3 run on flow cytometry with new Dispase lung dissociation protocol (gating below); and N=3 for histology/IHC. Data shown in FIG. 28B.

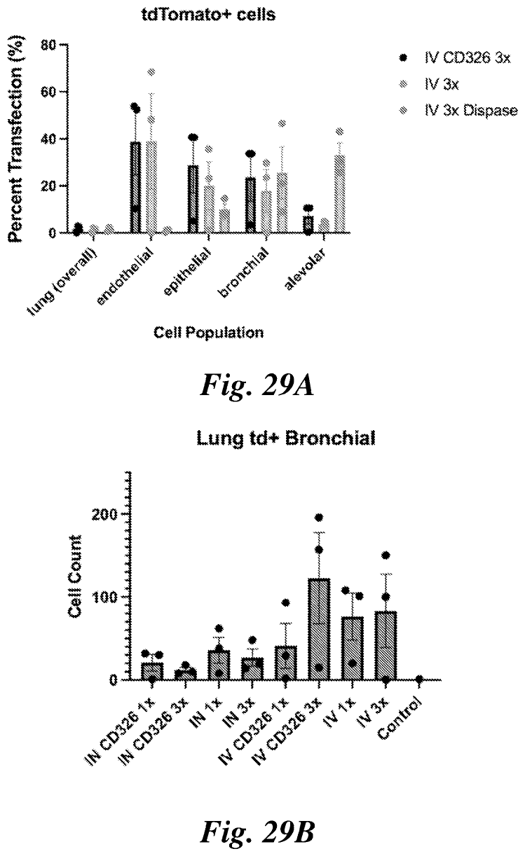

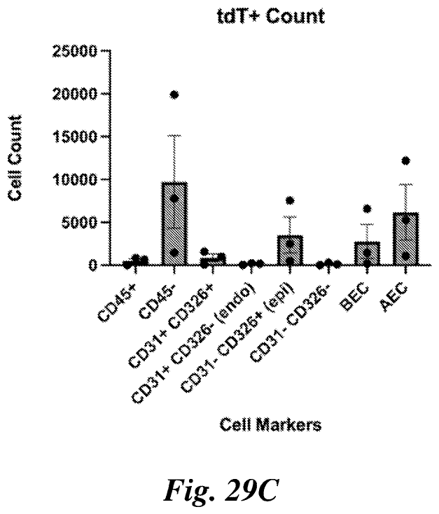

Referring now to FIG. 29A, FIG. 29B, and FIG. 29C, the Dispase method shows similar and more consistent results compared to previous 3× dosing. Similar results seen as in previous 3× injection studies. The new Dispase method showed markedly higher bronchial cells (182, 1477, 6599) achieved versus those observed previously (never higher than 200). Extremely low level of endothelial cells (max 239 cells vs averaging approximately 1500). Compared transfection across previous 3× treatment groups with percent of parent population (without live/dead stain): bronchial cells approximately 20% transfection; alveolar increasing drastically (ANOVA says NS); and extreme drop off in endothelial cell transfection.

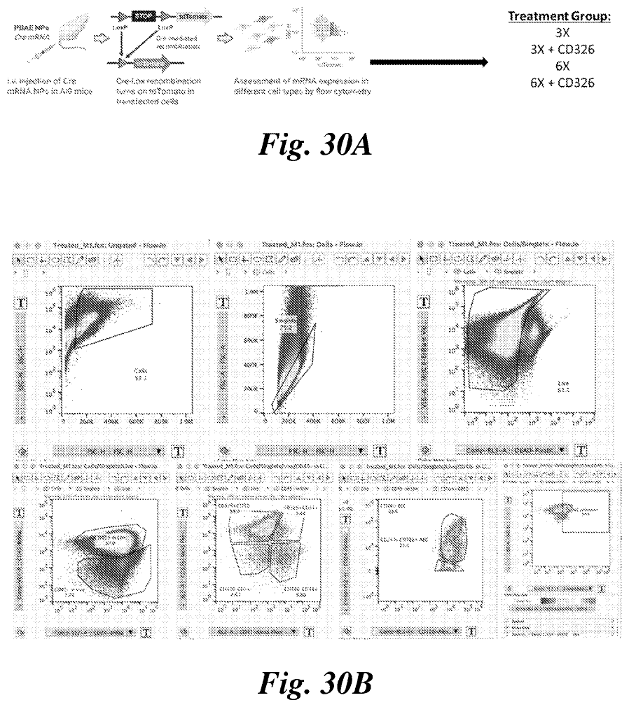

Referring now to FIG. 30A and FIG. 30B, Ai9 mouse data at dosages of 3×, 3×+CD326, 6×, and 6×+CD326 are provided.

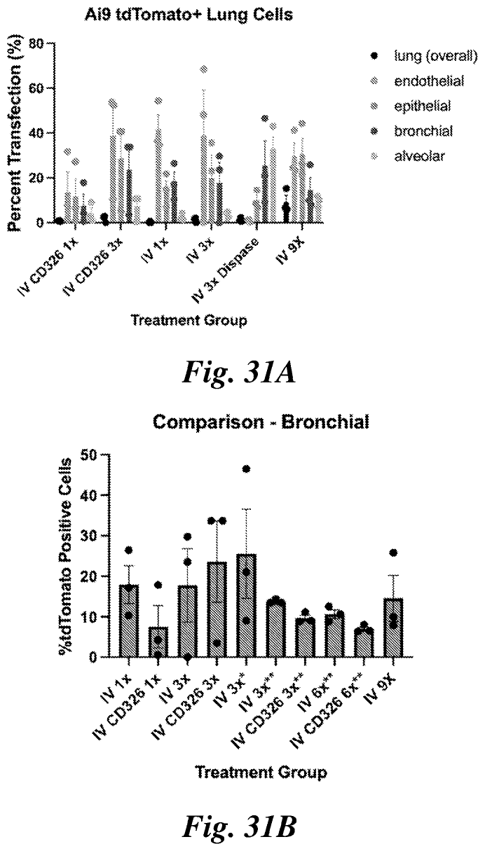

Additional Ai9 mouse data using a first Dispase protocol*, a second Dispase protocol **, or Green protocol (no *) for lung dissociation (IV 1×, CD326 1×, 3×, CD326 3×, 9×) are shown in FIG. 31. In this Example, the following dosing protocol was followed: 3× dosing (N=3); 3× dosing with CD326 targeting ligand (N=3); 6× dosing (N=3); 6× dosing with CD326 targeting ligand (N=3). In general, approximately 20% transfection of bronchial cells and 7-10% transfection of epithelial cells was observed. Transfection of immune, endothelial cells are at slightly lower levels than previously observed, but higher levels than the previous study. Also multiple dosages seems to have affected these cell populations more. The 3× vs 6× dosing does not appear to have made a statistically significant difference in the epi/bronchial cells, similar to what was observed with 9×. Without wishing to be bound to any one particular theory, it is thought that certain cell populations are saturated around 3×.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Cutting G R. Cystic fibrosis genetics: from molecular understanding to clinical application. Nat Rev Genet. 2015; 16 (1): 45-56.
Joynt A T, Kavanagh E W, Newby G A, Mitchell S, Eastman A C, Paul K C, Bowling A D, Osorio D L, Merlo C A, Patel S U, Raraigh K S, Liu D R, Sharma N, Cutting G R. Protospacer modification improves base editing of a canonical splice site variant and recovery of CFTR function in human airway epithelial cells. Mol. Ther. Nucleic Acids, Vol. 33, 335-350, 2023.
Hao Y, Hao S, Andersen-Nissen E, Mauck W M 3rd, Zheng S, Butler A, Lee M J, Wilk A J, Darby C, Zager M, Hoffman P, Stoeckius M, Papalexi E, Mimitou E P, Jain J, Srivastava A, Stuart T, Fleming L M, Yeung B, Rogers A J, McElrath J M, Blish C A, Gottardo R, Smibert P, Satija R. Integrated analysis of multimodal single-cell data. Cell 2021; Vol. 184, 13:3573-3587.e29.
M. Macek, A. Mackova, A. Hamosh, B. C. Hilman, R. F. Selden, G. Lucottej, K. J. Friedman, M. R. Knowles, B. J. Rosenstein, G. R. Cutting1, et al., Identification of Common Cystic Fibrosis Mutations in African-Americans with Cystic Fibrosis Increases the Detection Rate to 75%, 1997.
N. Sharma, T. A. Evans, M. J. Pellicore, E. Davis, M. A. Aksit, A. F. McCague, A. T. Joynt, Z. Lu, S. T. Han, A. F. Anzmann, et al., PLOS Genetics 2018, 14, e1007723.
A. Eid, S. Alshareef, M. M. Mahfouz, Biochemical Journal 2018, 475, 1955.
A. C. Komor, Y. B. Kim, M. S. Packer, J. A. Zuris, D. R. Liu, Nature 2016, 533, 420.
F. P. Polack, S. J. Thomas, N. Kitchin, J. Absalon, A. Gurtman, S. Lockhart, J. L. Perez, G. Pérez Marc, E. D. Moreira, C. Zerbini, et al., New England Journal of Medicine 2020, 383, 2603.
L. R. Baden, H. M. el Sahly, B. Essink, K. Kotloff, S. Frey, R. Novak, D. Diemert, S. A. Spector, N. Rouphael, C. B. Creech, et al., New England Journal of Medicine 2021, 384, 403.
Y. Rui, D. Wilson, S. Tzeng, H. Yamagata, D. Sudhakar, M. Conge, C. Berlinicke, D. Zack, A. Tuesca J. Green, Science Advances 2022, Vol. 8, Issue 1.
O. Moran, O. Zegarra-Moran, Journal of Cystic Fibrosis 2008, 7, 483.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nanoparticle comprising at least one DNA or mRNA molecule comprising a nucleic acid sequence encoding a gene-editing protein and a polymer of formula (I): 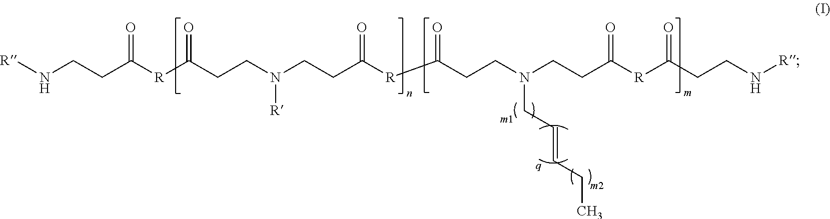
wherein:
m and n are each independently an integer from 1 to 10,000;
m1 is an integer selected from 0, 1, 2, 3, 4, 5, 6, 7, 8, 9, and 10;
m2 is an integer selected from the group consisting of 1, 2, 3, 4, 5, 6, 7, 8, 9, 10, 11, 12, 13, 14, 15, 16, 17, 18, 19, and 20;
q is an integer selected from 0 or 1;
wherein:
—(CH₂)ₘ₁—(C═C)_{q}—(CH₂)ₘ₂—CH₃ comprises a hydrophobic sidechain;
R comprises a divalent radical comprising a biodegradable ester linkage and/or a bioreducible disulfide linkage;
R′ is hydrophilic sidechain comprising a monovalent radical derived from a hydrophilic amine monomer;
R″ is monovalent radical derived from an amine-containing end capping group; and
pharmaceutically acceptable salts thereof.

2. The nanoparticle of claim 1, wherein R is selected from: 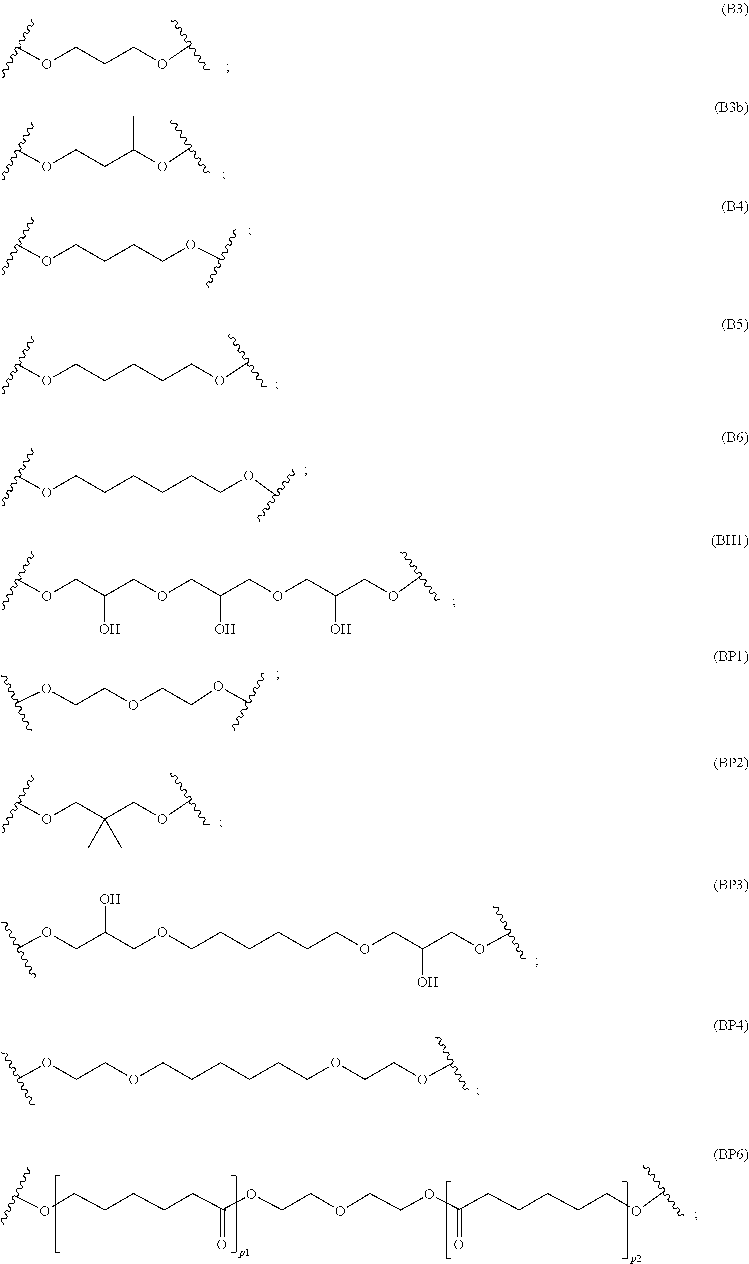 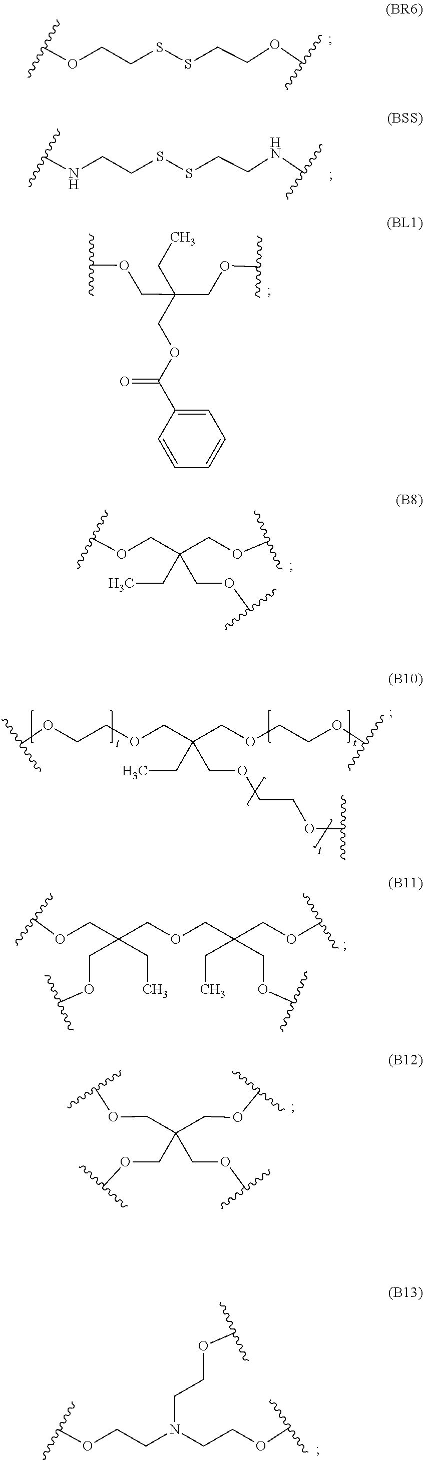
wherein each p1, p2, and t is independently an integer selected from the group consisting of 1, 2, 3, 4, 5, 6, 7, 8, 9, and 10.

3. The nanoparticle of claim 1, wherein R′ is selected from:

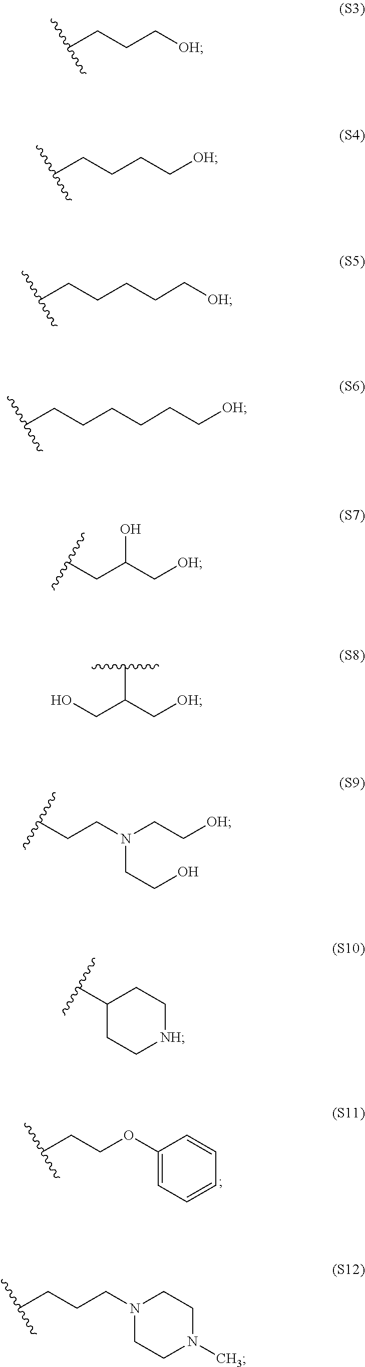

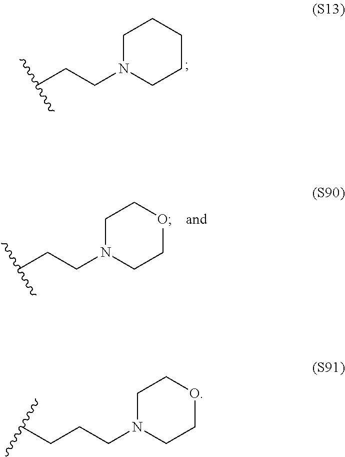 [SMILES: *CCN1CCCCC1 ; *CCN1CCOCC1 ; "and" ; *CCCN1CCOCC1]

4. The nanoparticle of claim 1, wherein R″ is selected from:

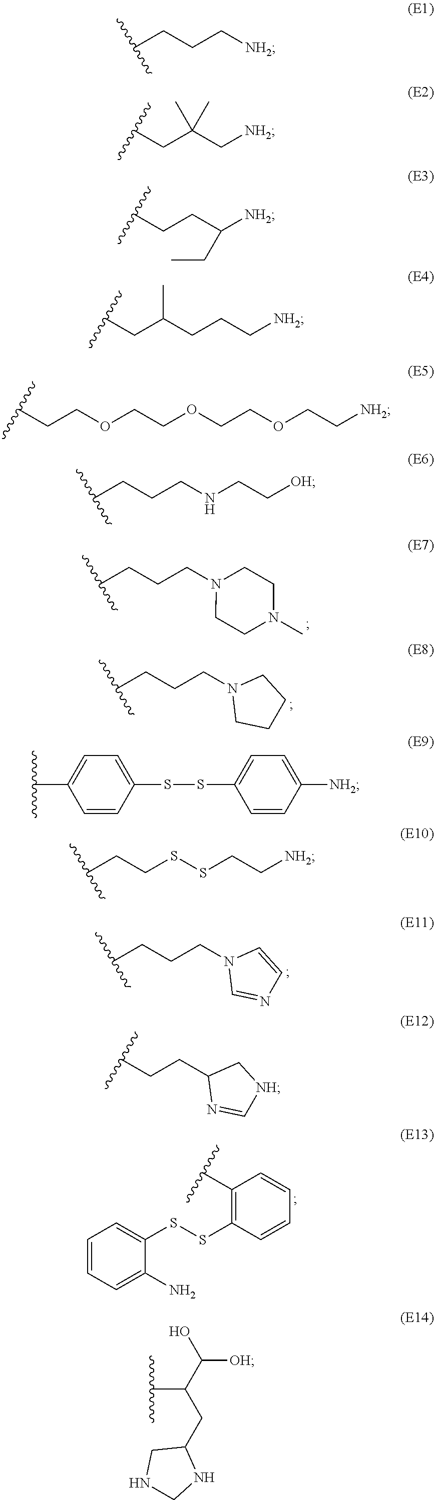

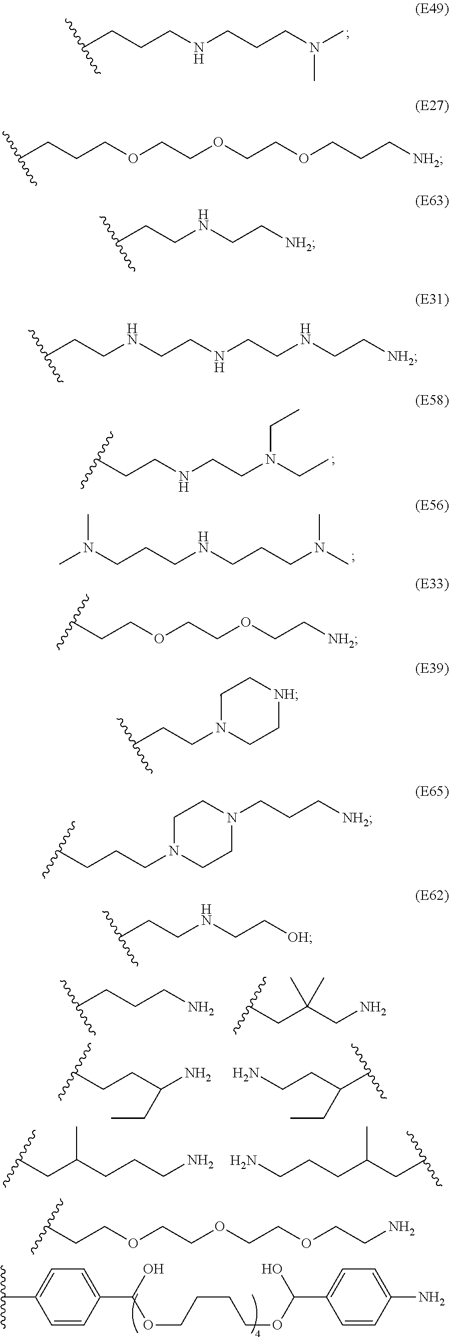

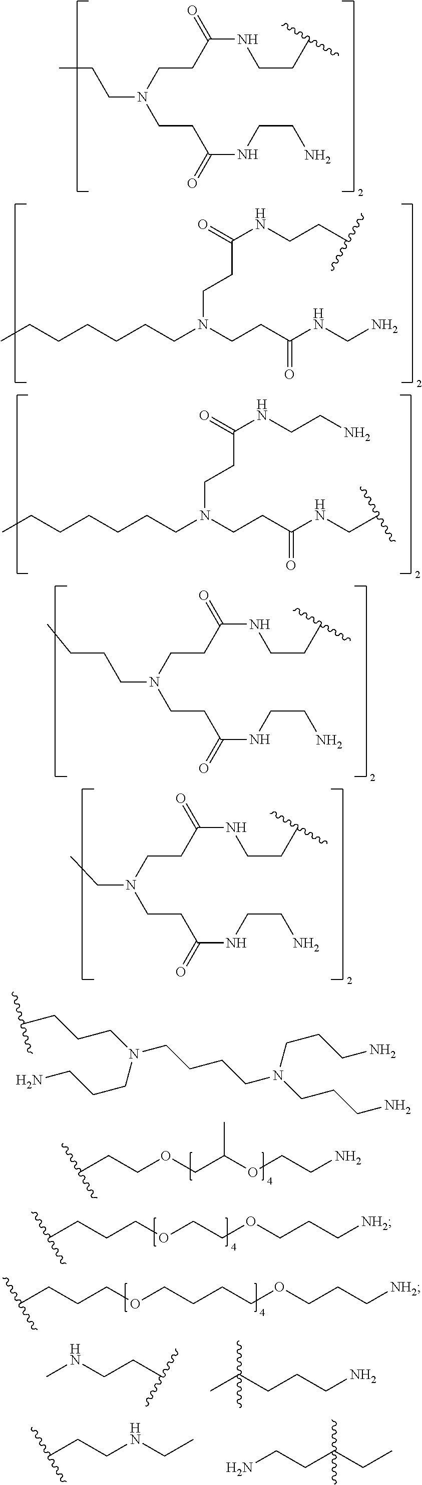

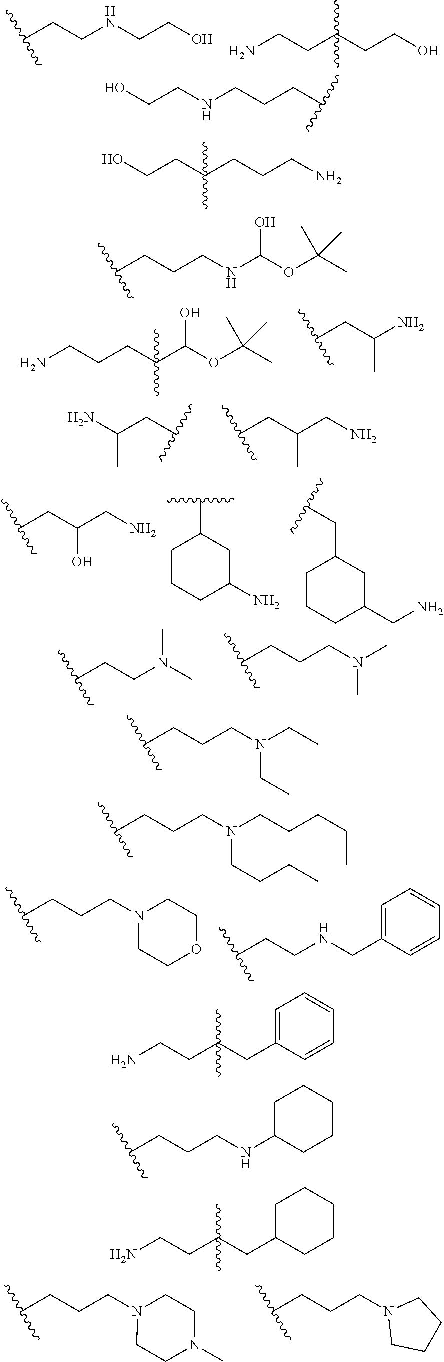

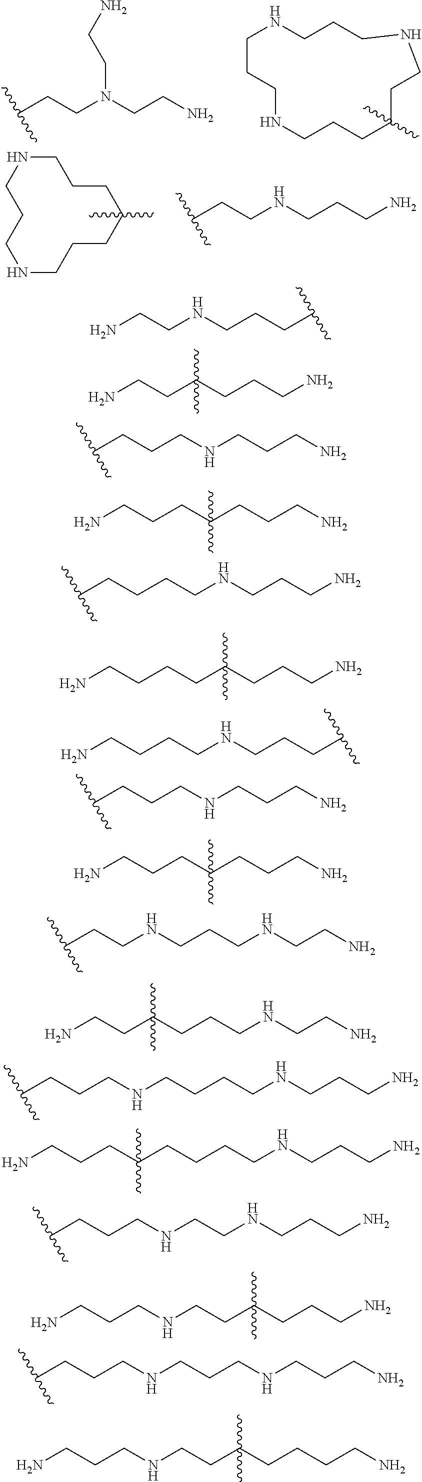

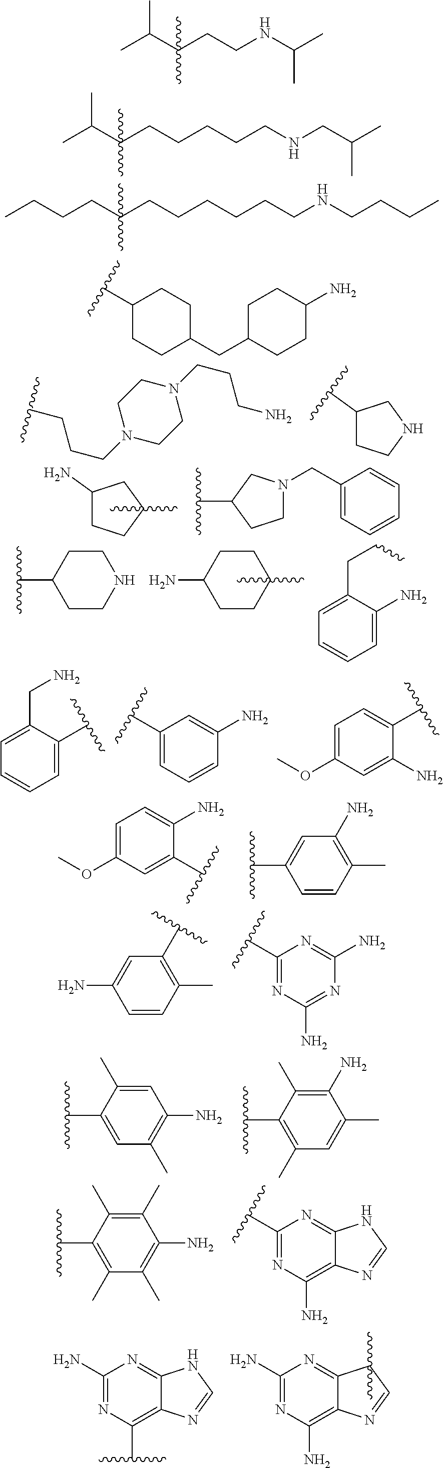

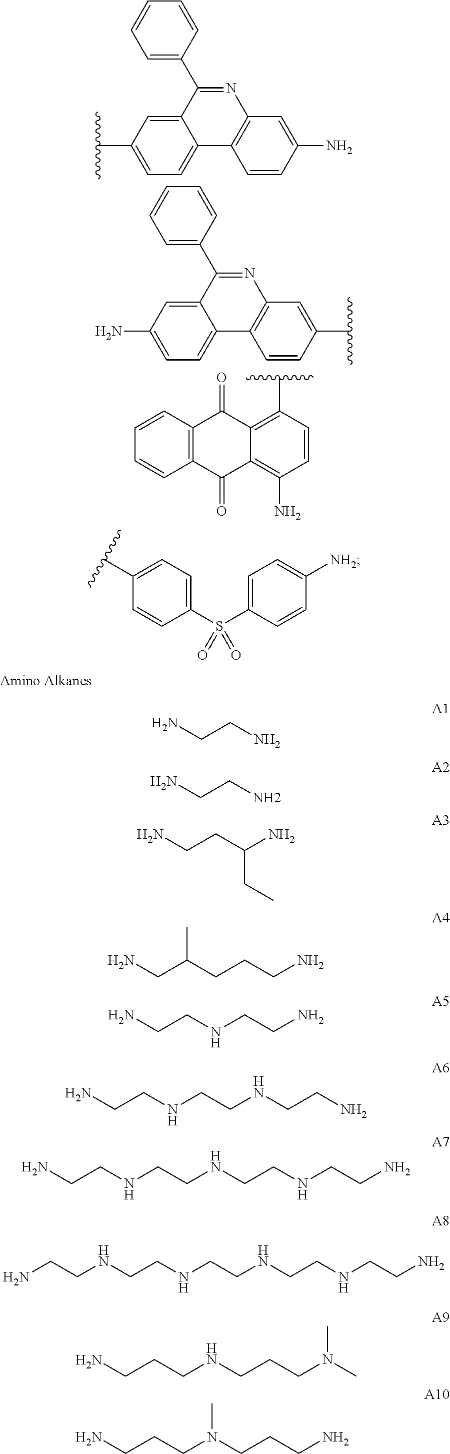

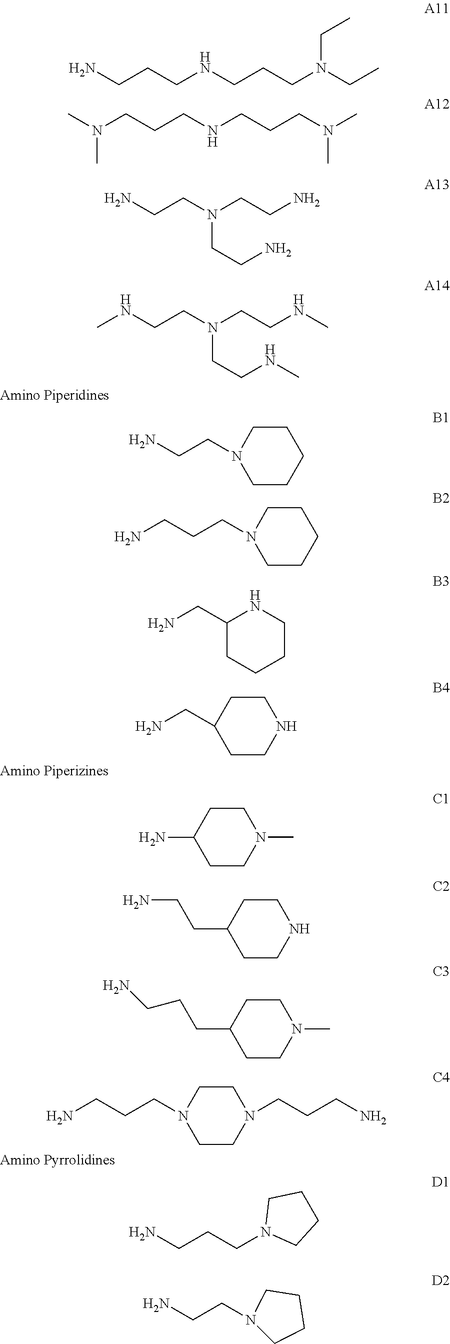

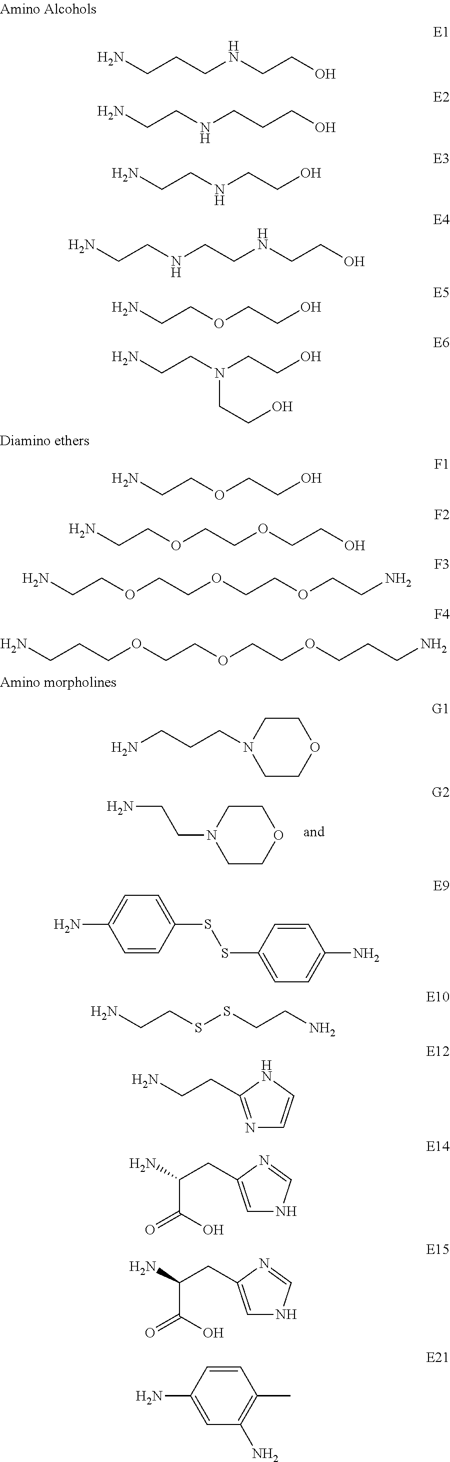

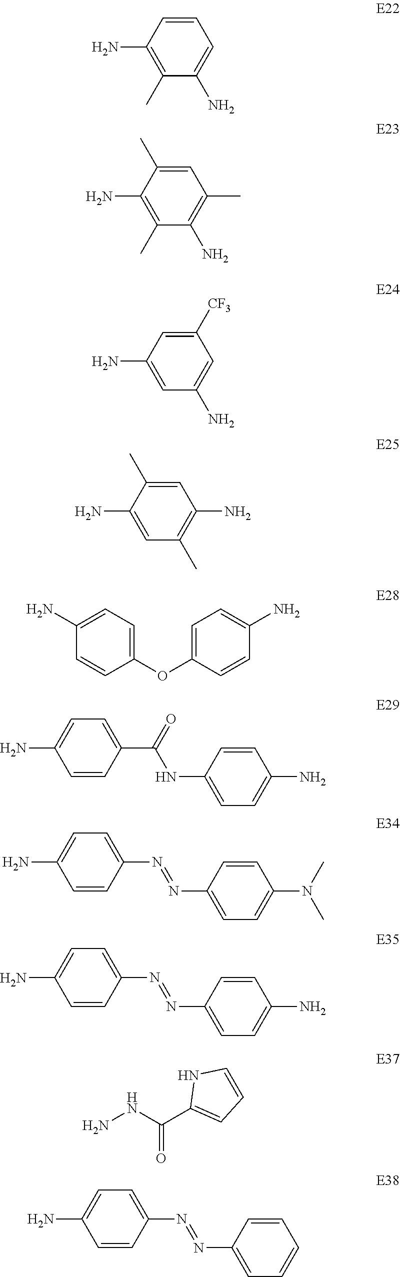

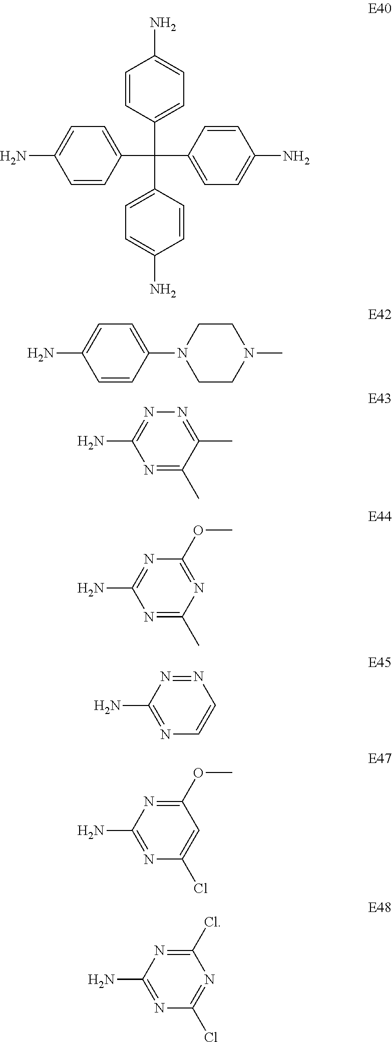

5. The nanoparticle of claim 1, wherein —(CH₂)ₘ₁—(C═C)_{q}—(CH₂)ₘ₂—CH₃ is selected from:

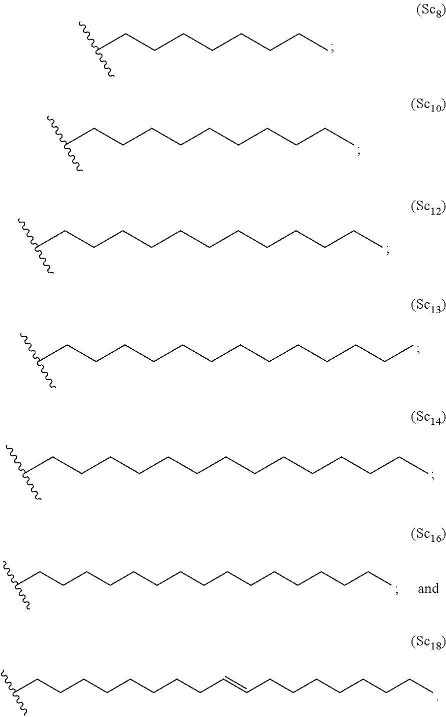

6. The nanoparticle of claim 1, wherein the gene-editing protein is selected from the group consisting of CRISPR-associated nuclease, Cre recombinase, Flp recombinase, a meganuclease, a Transcription Activator-Like Effector Nuclease (TALEN), a Zinc-Finger Nuclease (ZFN), or a natural or engineered variant, family-member, orthologue, fragment or fusion construct thereof.

7. The nanoparticle of claim 6, wherein the gene-editing protein comprises a Cas9 endonuclease.

8. The nanoparticle of claim 7, further comprising a gRNA or DNA encoding a gRNA.

9. The nanoparticle of claim 8, wherein the Cas9 endonuclease and the gRNA are encoded on the same plasmid or wherein the Cas9 endonuclease and the gRNA are encoded on different plasmids.

10. (canceled)

11. The nanoparticle of claim 1, comprising an mRNA molecule.

12. The nanoparticle of claim 11, comprising a polymer-to-mRNA weight-to-weight ratios having a range from about 100:1 to about 5:1.

13. The nanoparticle of claim 12, wherein the polymer-to-mRNA weight-to-weight ratio is selected from 60:1, 50:1, 40:1, 30:1, and 20:1.

14. The nanoparticle of claim 1, wherein the polymer of formula (I) has the following chemical structure having terminal amine end groups:

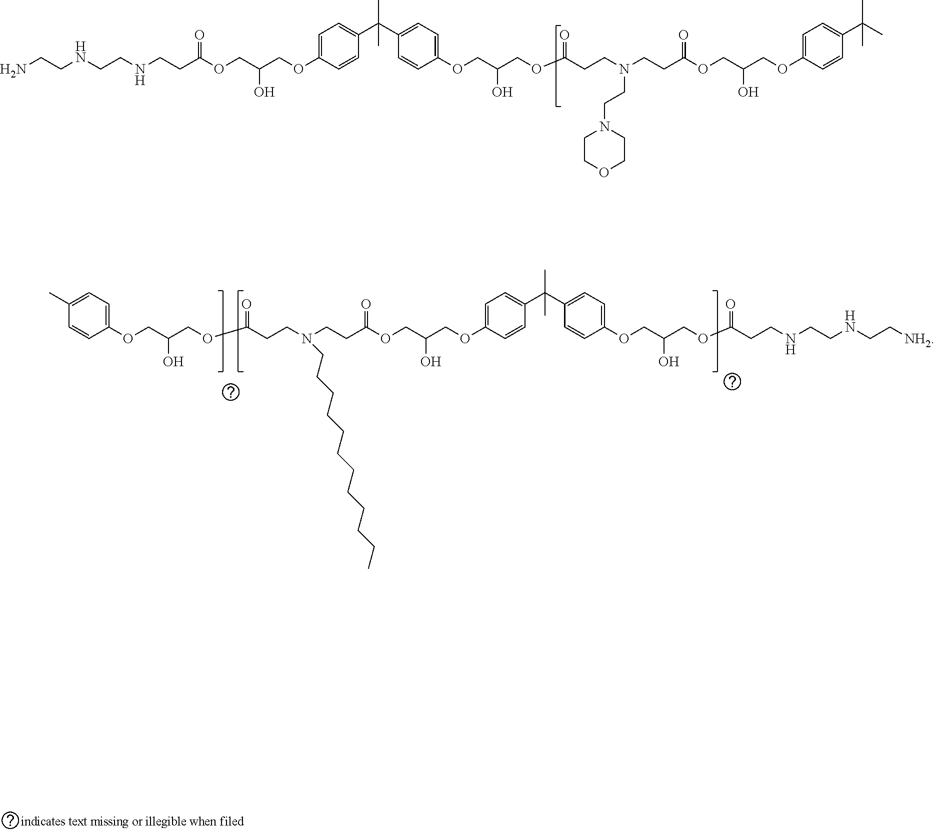

15. The nanoparticle of claim 14, wherein the terminal amine end groups are replaced by a monomer selected from E1, E39, and E58.

16. The nanoparticle of claim 1, wherein n and m are each independently an integer having a range from 1 to 10,000, 1 to 1,000, 1 to 100, 1 to 30, 1 to 20, 1 to 15, and 1 to 10, 1 to 5, 1 to 4, 1 to 3, 1 to 2, and 1.

17. The nanoparticle of claim 1, wherein the nanoparticle has a size between about 100 nm to about 150 nm in diameter.

18. The nanoparticle of claim 1, further comprising a poly(ethylene glycol) (PEG)-lipid.

19. The nanoparticle of claim 1, further comprising an antibody targeting one or more molecules expressed on lung epithelial cells.

20. A method for gene editing, comprising contacting a cell with the nanoparticle of claim 1.

21. The method of claim 20, wherein the gene-editing protein directs site-specific target DNA disruption, mutation, deletion, or repair.

22. The method of claim 20, wherein;
(a) the nanoparticle and cell are contacted in vivo;
(b) the nanoparticle and cell are contacted ex vivo; and/or
(c) the cell is a mammalian cell.

23-24. (canceled)

25. The method of claim 22, wherein the mammalian cell is a human cell.

26. A method of treating a genetic disease, condition, or disorder, the method comprising administering a therapeutically effective amount of a nanoparticle of claim 1 to a subject in need of treatment thereof.

27. The method of claim 26, wherein the disease, condition, or disorder comprises cystic fibrosis (CF).

28. The method of claim 27, wherein the subject being treated for cystic fibrosis has a canonical splice site variant.

29. The method of claim 28, wherein the canonical splice site variant:
(a) introduces a premature stop codon (PTC);
(b) comprises an alteration of a 5′GT splice motif or a 3′AG splice motif; and/or
(c) comprises c.2988+1G>A.

30-31. (canceled)

32. The method of claim 29, wherein the treatment corrects c.2988+1G>A in airway epithelia of the subject.

33. The method of claim 27, wherein the subject is of African descent.

34. The method of claim 27, further comprising administering a protein-targeted modulator therapy.

35. The method of claim 27, comprising a systemic administration of the nanoparticle.

36. The method of claim 35, wherein the systemic administration is intravenous (IV) or intranasal (IN).
